(19) United States
(12) Patent Application Publication
(10) Pub. No.: US 2006/0088532 A1
(43) Pub. Date: Apr. 27, 2006

(54) Lymphatic and blood endothelial cell genes

(76) Inventors: Kari Alitalo, Helsinki (FI); Taija Makinen, Helsinki (FI); Tatiana Petrova, Helsinki (FI); Pipsa Saharinen, Helsinki (FI); Juha Saharinen, Helsinki (FI)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/505,928

(22) PCT Filed: Mar. 7, 2003
(86) PCT No.: PCT/US03/06900

§ 371 Date: Sep. 8, 2005

Related U.S. Application Data
(60) Provisional application No. 60/363,019, filed on Mar. 7, 2002.

(51) Int. Cl. A61K 48/00; A61K 39/395; A61K 38/18

(52) U.S. Cl. 424/145.1; 514/2; 514/44

(57) ABSTRACT

The invention provides polynucleotides and genes that are differentially expressed in lymphatic versus blood vascular endothelial cells. These genes are useful for treating diseases involving lymphatic vessels, such as lymphedema, various inflammatory diseases, and cancer metastasis via the lymphatic system.

### BACKGROUND OF THE INVENTION

1. Field of the Invention

The invention relates to polynucleotides and proteins specifically expressed in lymphatic endothelial cells.

2. Description of the Related Art

Recent evidence on the association of lymphangiogenic growth factors with intralymphatic growth and metastasis of cancers (Mandriota, et al., EMBO J. 20:672-682 (2001); Skobe, et al., Nat. Med. 7:192-198 (2001); Stacker, et al., Nat. Med. 7:186-191 (2001);. Karpanen, et al., Cancer Res. 61:1786-1790 (2001)) has raised hopes that lymphatic vessels could be used as an additional target for tumor therapy. Cancer cells spread within the body by direct invasion to surrounding tissues, spreading to body cavities, invasion into the blood vascular system (hematogenous metastasis), as well as spread via the lymphatic system (lymphatic metastasis). Regional lymph node dissemination is the first step in the metastasis of several common cancers and correlates highly with the prognosis of the disease. The lymph nodes that are involved in draining tissue fluid from the tumor area are called sentinel nodes, and diagnostic measures are in place to find these nodes and to remove them in cases of suspected metastasis. However, in spite of its clinical relevance, little is known about the mechanisms leading to metastasis via the bloodstream or via the lymphatics.

Until recently, the lymphatic vessels have received much less attention than blood vessels, despite their importance in medicine. Lymphatic vessels collect protein-rich fluid and white blood cells from the interstitial space of most tissues and transport them as a whitish opaque fluid, the lymph, into the blood circulation. Small lymphatic vessels coalesce into larger vessels, which drain the lymph through the thoracic duct into large veins in the neck region. Lymph nodes serve as filtering stations along the lymphatic vessels and lymph movement is propelled by the contraction of smooth muscles surrounding collecting lymphatic vessels and by bodily movements, the direction of flow being secured by valves as it is in veins. The lymphatic capillaries are lined by endothelial cells, which have distinct junctions with frequent large interendothelial gaps. The lymphatic capillaries also lack a continuous basement membrane, and are devoid of pericytes. Anchoring filaments connect the abluminal surfaces of lymphatic endothelial cells to the perivascular extracellular matrix and pull to maintain vessel patency in the presence of tissue edema. The absence or obstruction of lymphatic vessels, which is usually the result of an infection, surgery, or radiotherapy and in rare cases, a genetic defect, causes accumulation of a protein-rich fluid in tissues, lymphedema. The lymphatic system is also critical in fat absorption from the gut and in immune responses. Bacteria, viruses, and other foreign materials are taken up by the lymphatic vessels and transported to the lymph nodes, where the foreign material is presented to immune cells and where dendritic cells traverse via the lymphatics. There has been slow progress in the understanding of and ability to manipulate the lymphatic vessels.

Abnormal development or function of the lymphatic ECs can result in tumors or malformations of the lymphatic vessels, such as lymphangiomas or lymphangiectasis. Witte, et al., Regulation of Angiogenesis (eds. Goldber, I. D. & Rosen, E. M.) 65-112 (Birkauser, Basel, Switzerland, 1997). The VEGFR-3 tyrosine kinase receptor is expressed in the normal lymphatic endothelium and is upregulated in many types of vascular tumors, including Kaposi's sarcomas. Jussila, et al., Cancer Res 58, 1955-1604 (1998); Partanen, et al., Cancer 86:2406-2412 (1999). Absence or dysfunction of lymphatic vessels which can result from an infection, surgery, radiotherapy or from a genetic defect, causes lymphedema, which is characterized by a chronic accumulation of protein-rich fluid in the tissues that leads to swelling. The importance of VEGFR-3 signaling for lymphangiogenesis was revealed in the genetics of familial lymphedema, a disease characterized by a hypoplasia of cutaneous lymphatic vessels, which leads to a disfiguring and disabling swelling of the extremities. Witte, et al., Regulation of Angiogenesis (eds. Goldber, I. D. & Rosen, E. M.) 65-112 (Birkauser, Basel, Switzerland, 1997); Rockson, S. G, Am. J. Med. 110, 288-295 (2001). Some members of families with lymphoedema are heterozygous for missense mutations of the VEGFR3 exons encoding the tyrosine kinase domain, which results in an inactive receptor protein. Karkkainen, et al., Nature Genet. 25:153-159 (2000); Irrthum, et al., Am. J. Hum. Genet. 67:295-301 (2000).

There is a need in the art for information on the transcriptional program which controls the diversity of endothelial cells, and into the mechanisms of angiogenesis and lymphangiogenesis. There is also a need in the art for new vascular markers, which may be used as valuable targets in the study of a number of diseases involving the lymphatic vessels, including tumor metastasis.

### SUMMARY OF THE INVENTION[TO BE REVISED UPON FINALIZATION OF CLAIMS]

The compositions of the present invention include isolated polynucleotides, in particular, lymphatic endothelial genes, polypeptides, isolated polypeptides encoded by these polynucleotides, recombinant DNA molecules, cloned genes or degenerate variants thereof, especially naturally occurring variants such as allelic variants, and antibodies that specifically recognize one or more epitopes present on such polypeptides.

The compositions of the present invention additionally include vectors, including expression vectors, containing the polynucleotides of the invention, cells genetically engineered to contain such polynucleotides and cells genetically engineered to express such polynucleotides.

In selected embodiments, such isolated polynucleotides of the invention represent a polynucleotide comprising a nucleotide sequence set forth in the sequence listing, e.g., any of SEQ ID NOS:1-30.

The polynucleotides of the present invention also include, but are not limited to, a polynucleotide that hybridizes to the complement of the nucleotide sequence of SEQ ID NOS:1-30 under highly stringent hybridization conditions; a polynucleotide that hybridizes to the complement of the nucleotide sequence of SEQ ID NOS:1-30 under moderately stringent hybridization conditions; a polynucleotide which is an allelic variant of any polynucleotide recited above; a polynucleotide which encodes a species homologue of any of the proteins recited above; of a polynucleotide that encodes a polypeptide comprising a specific domain or truncation of the polypeptide encoded by any one of SEQ ID NOS:1-30. Exemplary high stringency hybridization conditions are hybridization at 42° C. for 20 hours in a solution containing 50% formamide, 5×SSPE, 5× Denhardt's solution, 0.1% SDS and 0.1 mg/ml denatured salmon sperm DNA, with a wash in 1×SSC, 0.1% SDS for 30 minutes at 65° C.

Another aspect of the invention is drawn to LEC and BEC polypeptides, including polypeptides encoded by the polynucleotides described above. In some embodiments, the polypeptides are the mature forms of the polypeptides of the invention. Expressly contemplated is a purified and isolated polypeptide comprising an amino acid sequence selected from the group consisting of SEQ ID NOS: 31-44, 46, 48, 50, 52, 81, 187, 207, 211, 221, 235, 241, 293, and 391; and a purified and isolated polypeptide comprising an amino acid sequence selected from the group consisting of: (a) SEQ ID NOS: 31-34, 46, 48, 207, 676, 859, and 861; and (b) an extracellular domain fragment of at least 10 amino acids of an amino acid sequence of (a). Further, this aspect of the invention includes a purified and isolated, soluble polypeptide as described immediately above, comprising an extracellular domain fragment of an amino acid sequence selected from the group consisting of: SEQ ID NOS: 31-34, 46, 48, 207, 676, 859, and 861, wherein the polypeptide lacks any transmembrane domain. Such a polypeptide may further lack any intracellular domain. Also, the invention contemplates a fusion protein comprising a polypeptide as described above fused to an immunoglobulin fragment comprising an immunoglobulin constant region.

In a related aspect, the invention provides a composition comprising a polypeptide or protein as described above and a pharmaceutically acceptable diluent, carrier or adjuvant; Polypeptide compositions of the invention may comprise an acceptable carrier, such as a hydrophilic, e.g., pharmaceutically acceptable, carrier. Further provided is a kit comprising such a composition and a protocol for administering the pharmaceutical composition to a mammalian subject to modulate the lymphatic system in the subject. The invention also provides an antibody that specifically binds to a polypeptide as described above, and that antibody is humanized in some embodiments. Still further, the invention provides a protein comprising an antigen binding domain of an antibody that specifically binds to a polypeptide as described hereinabove, wherein the protein specifically binds to the polypeptide.

The invention also relates to methods for producing a polypeptide comprising growing a culture of the cells of the invention in a suitable culture medium, and purifying the protein from the culture or from an extract of the cells. In particular, the invention contemplates a method for producing a LEC polypeptide comprising steps of growing a host cell transformed or transfected with an expression vector as described herein under conditions in which the cell expresses the polypeptide encoded by the polynucleotide.

Methods of identifying the products and compositions described herein are also provided by the invention. In particular, the invention provides a method of identifying a LEC nucleic acid comprising: (a) contacting a biological sample containing a candidate LEC nucleic acid with a polynucleotide comprising a fragment of at least 14 contiguous nucleotides of a sequence selected from the group consisting of SEQ ID NOS:1-30, 45, 47, 49, 51, 82, 93, 111, 188, 208, 212, 236, 242, 294, and 392, or a complement thereof, under the following stringent hybridization conditions: (i) hybridization at 42° C. for 20 hours in a solution containing 50% formamide, 5×SSPE, 5× Denhardt's solution, 0.1% SDS and 0.1 mg/ml denatured salmon sperm DNA, and (ii) washing for 30 minutes at 65° C. in 1×SSC, 0.1% SDS; and (b) detecting hybridization of the candidate LEC nucleic acid and the polynucleotide, thereby identifying a LEC nucleic acid.

The invention also provides a method of identifying a LEC protein comprising: (a) contacting a biological sample containing a candidate LEC protein with a LEC protein binding partner selected from the group consisting of an antibody as described herein or a protein or polypeptide as described herein, under conditions suitable for binding therebetween; and (b) detecting binding between the candidate LEC protein and the LEC binding partner, thereby identifying a LEC protein.

Another related aspect of the invention is a method of identifying a LEC comprising: (a) contacting a biological sample comprising cells with a LEC binding partner under conditions suitable for binding therebetween, wherein the LEC binding partner comprises an antibody that binds to a polypeptide comprising a sequence selected from the group consisting of SEQ ID NOS:31-34, 46, 48, 207, 676, 859, and 861, or comprises an antigen binding fragment of the antibody; and (b) identifying a LEC by detecting binding between a cell and the LEC binding partner, where binding of the LEC binding partner to the cell identifies a LEC.

Polynucleotides according to the invention have numerous applications in a variety of techniques known to those skilled in the art of molecular biology. These techniques include use as hybridization probes, use as primers for PCR, use for chromosome and gene mapping, use in the recombinant production of protein, and use in generation of anti-sense DNA or RNA, their chemical analogs and the like. For example, when the expression of an mRNA is largely restricted to a particular cell or tissue type, such as a lymphatic endothelial cell, polynucleotides of the invention can be used as hybridization probes to detect the presence of the particular cell or tissue mRNA in a sample using, e.g., in situ hybridization.

In another aspect, the invention provides a composition comprising an isolated polynucleotide that comprises a nucleotide sequence that encodes a polypeptide comprising an amino acid sequence selected from the group consisting of SEQ ID NOs: 31-44, 46, 48, 50, 52, 81, 187, 207, 211, 221, 235, 241, 293, and 391; and a pharmaceutically acceptable diluent, carrier or adjuvant. In some embodiments, the composition comprises a polynucleotide that comprises a nucleotide sequence selected from the group consisting of SEQ ID NOS: 14-30, 45, 47, 49, 51, 82, 93, 111, 188, 208, 212, 222, 236, 242, 294, and 392, or a fragment thereof that encodes the polypeptide.

Still another aspect of the invention is an expression vector comprising an expression control sequence operably linked to a polynucleotide that comprises a nucleotide sequence that encodes a polypeptide comprising an amino acid sequence selected from the group consisting of SEQ ID NOs: 31-44, 46, 48, 50, 52, 81, 187, 207, 211, 221, 235, 241, 293, and 391. In some embodiments, the expression vector is a replication-deficient adenoviral or adeno-associated viral vector containing the polynucleotide. A related aspect of the invention is a composition comprising an expression vector as described above and a pharmaceutically acceptable diluent, carrier, or adjuvant. Further, the invention provides a kit comprising the composition containing either the above-described polynucleotide or vector and a pharmaceutically acceptable diluent, carrier or adjuvant, packaged with a protocol for administering the composition to a mammalian subject to modulate the lymphatic system in the subject.

The invention further provides a host cell transformed or transfected with an expression vector as described above.

The polypeptides according to the invention can be used in a variety of conventional procedures and methods that are currently applied to other proteins. In addition, a polypeptide of the invention can be used to generate an antibody that specifically binds the polypeptide.

In one aspect of the invention, a method is provided for differentially modulating the growth or differentiation of blood endothelial cells (BEC) or lymphatic endothelial cells (LEC), comprising contacting endothelial cells with a composition comprising an agent that differentially modulates blood or lymphatic endothelial cells, said agent selected from the group consisting of: (a) a polypeptide that comprises an amino acid sequence of a BEC polypeptide or a LEC polypeptide, or an active fragment of the polypeptide; (b) a polynucleotide that comprises a nucleotide sequence that encodes a polypeptide according to (a); (c) an antibody that specifically binds to a polypeptide according to (a); (d) a polypeptide comprising a fragment of the antibody of (c), wherein the fragment and the antibody bind to the polypeptide; (e) an antisense nucleic acid to a human gene or mRNA encoding the polypeptide of (a); (f) an interfering RNA (RNAi) to a human gene or mRNA encoding the polypeptide of (a). The method may involve endothelial cell contact with the composition ex vivo or in vivo. The composition may comprise a pharmaceutically acceptable diluent, adjuvant, or carrier, and the contacting step may comprise administering the composition to a mammalian subject to differentially modulate BECs or LECs in the mammalian subject.

Further, the method may comprise identifying a human subject with a disorder characterized by hyperproliferation of LECs; and administering to the human subject the composition, wherein the agent differentially inhibits LEC growth compared to BEC growth; alternatively the method may comprise identifying a human subject with a disorder characterized by hyperproliferation of LECs; screening LECs of the subject to identify overexpression of a polypeptide set forth in Table 3; and administering to the human subject the composition, wherein the agent differentially inhibits LEC growth compared to BEC growth by inhibiting expression of the polypeptide identified by the screening step.

This aspect of the invention also contemplates a method of modulating the growth of lymphatic endothelial cells in a human subject, comprising steps of identifying a human subject with a hypoproliferative lymphatic disorder; screening the subject to identify underexpression or underactivity of a LEC polypeptide set forth in Table 3, wherein the protein is not set forth in Table 1 or 2; administering to the human subject the composition, wherein the agent comprises the LEC polypeptide (a) identified by the screening step or an active fragment of the polypeptide, or comprises the polynucleotide (b) that comprises a nucleotide sequence that encodes the polypeptide.

A related aspect of the invention is drawn to a use of an agent for the manufacture of a medicament for the differential modulation of blood vessel endothelial cell (BEC) or lymphatic vessel endothelial cell (LEC) growth or differentiation, the agent selected from the group consisting of: (a) a polypeptide that comprises an amino acid sequence of a BEC polypeptide or a LEC polypeptide, or an active fragment of the polypeptide; (b) a polynucleotide that comprises a nucleotide sequence that encodes a polypeptide according to (a); (c) an antibody that specifically binds to a polypeptide according to (a); (d) a polypeptide comprising a fragment of the antibody of (c), wherein the fragment and the antibody bind to the polypeptide; (e) an antisense nucleic acid to a human gene or mRNA encoding the polypeptide of (a); (f) an interfering RNA (RNAi) to a human gene or mRNA encoding the polypeptide of (a).

In another aspect, the invention provides a method of identifying compounds that modulate growth of endothelial cells, comprising culturing endothelial cells in the presence and absence of a compound; and measuring expression of at least one BEC or LEC gene in the cells, wherein the BEC or LEC gene is selected from the genes encoding polypeptides set forth in Tables 3 and 4, wherein a change in expression of at least one BEC gene in the presence compared to the absence of the compound identifies the compound as a modulator of BEC growth, and wherein a change in expression of at least one LEC gene in the presence compared to the absence of the compound identifies the compound as a modulator of LEC growth. The method may be used to screen for a compound that selectively modulates BEC or LEC growth or differentiation, wherein the measuring step comprises measuring expression of at least one BEC gene and at least one LEC gene in the cells, and wherein the method comprises screening for a compound that selectively modulates BEC or LEC growth or differentiation by selecting a compound that differentially modulates expression of the at least one BEC gene compared to expression of the at least one LEC gene.

Further, the invention comprehends a method or use according to the aspects of the invention described above, wherein the polypeptide is a LEC polypeptide selected from the LEC polypeptides set forth in Table 3, and the agent differentially modulates LEC growth or differentiation over BEC growth or differentiation. In some embodiments, the LEC polypeptide comprises an amino acid sequence selected from the group consisting of SEQ ID NOs: 81, 187, 207, 211, 221, 235, 241, 293, and 391; in other embodiments, the LEC polypeptide comprises an amino acid sequence selected from the group consisting of SEQ ID NOs: 31-34, 46, and 48. In these embodiments, an agent may be an antibody that specifically binds to a LEC polypeptide as described above, or a polypeptide fragment of such an antibody. Further, the agent may be an extracellular domain of a polypeptide described above, a polynucleotide encoding an extracellular domain, or an antisense molecule or nucleic acid. Alternatively, the polypeptide is a BEC polypeptide selected from the BEC polypeptides set forth in Table 4, and the agent differentially modulates BEC growth or differentiation over LEC growth or differentiation. Preferably, the polypeptides are not set forth in Tables 1 or 2.

The methods of the present invention further relate to methods for detecting the presence of the polynucleotides or polypeptides of the invention in a sample. Such methods can, for example, be utilized as part of prognostic and diagnostic evaluation of disorders as recited above and for the identification of subjects exhibiting a predisposition to such conditions. Furthermore, the invention provides methods for evaluating the efficacy of drugs, and monitoring the progress of patients, involved in clinical trials for the treatment of disorders related to lymphatic endothelial cells.

The invention also provides methods for the identification of compounds that modulate the expression of the polynucleotides and/or polypeptides of the invention. Such methods can be utilized, for example, for the identification of compounds that can ameliorate symptoms of disorders related to expression of proteins encoded by any one of SEQ ID NOS:1-30 as recited above. Such methods can include, but are not limited to, assays for identifying compounds and other substances that interact with (e.g., bind to) the polypeptides of the invention.

Further, the invention provides a method of assaying for risk of developing hereditary lymphedema, comprising (a) assaying nucleic acid of a human subject for a mutation that correlates with a hereditary lymphedema phenotype and alters the encoded amino acid sequence of at least one gene allele of the human subject when compared to the amino acid sequence of the polypeptide encoded by a corresponding wild-type gene allele, wherein the wild-type polypeptide is a polypeptide identified in Table 3. Alternatively, a method of assaying for risk of developing hereditary lymphedema, comprises (a) assaying nucleic acid of a human subject for a mutation that correlates with a hereditary lymphedema phenotype and alters the encoded amino acid sequence of at least one gene allele of the human subject when compared to the amino acid sequence of the polypeptide encoded by a corresponding wild-type gene allele, wherein the wild-type polypeptide comprises an amino acid sequence selected from the group consisting of SEQ D NOS: 31-44, 46, 48, 52, 54, 207, 676, 859, and 861; (b) correlating the presence or absence of the mutation in the nucleic acid to a risk of developing hereditary lymphedema, wherein the presence of the mutation in the nucleic acid correlates with an increased risk of developing hereditary lymphedema, and wherein the absence of the mutation in the nucleic acid correlates with no increased risk of developing hereditary lymphedema.

In another method of assaying for risk of developing hereditary lymphedema, the steps comprise (a) assaying nucleic acid of a human subject for a mutation that alters the encoded amino acid sequence of at least one transcription factor allele of the human subject and alters transcription modulation activity of the transcription factor polypeptide encoded by the allele, when compared to the transcription modulation activity of a transcription factor polypeptide encoded by a wild-type allele, wherein the wild-type transcription factor polypeptide comprises an amino acid sequence selected from the group consisting of SEQ ID NO: 81, SEQ ID NO: 211, SEQ ID NO: 241, and transcription factors encoded by sequences in Table 5; and (b) correlating the presence or absence of the mutation in the nucleic acid to a risk of developing hereditary lymphedema, wherein the presence of the mutation in the nucleic acid correlates with an increased risk of developing hereditary lymphedema, and wherein the absence of the mutation in the nucleic acid correlates with no increased risk of developing hereditary lymphedema. In this method, the wild-type transcription factor allele may comprise the Sox18 amino acid sequence set forth as SEQ ID NO:54. In some embodiments of this method, the assaying identifies a mutation altering a transactivating or DNA binding domain amino acid sequence of the protein encoded by the Sox18 allele; in some other embodiments of the method, the mutation reduces transcriptional activation of a SOX18-responsive gene compared to transcriptional activation of the gene by wild-type SOX18.

In a related aspect, the invention provides a method of assaying for risk of developing hereditary lymphedema, comprising (a) assaying nucleic acid of a human subject for a mutation that alters the encoded amino acid sequence of at least one LEC gene allele of the human subject and alters the binding affinity of the adhesion polypeptide encoded by the LEC gene allele, when compared to the binding affinity of an adhesion polypeptide encoded by a wild-type allele, wherein the wild-type adhesion polypeptide comprises an amino acid sequence selected from the group consisting of SEQ ID NOS:31-34, 46, 207, 676, 859, and 861; and (b) correlating the presence or absence of the mutation in the nucleic acid to a risk of developing hereditary lymphedema, wherein the presence of the mutation in the nucleic acid correlates with an increased risk of developing hereditary lymphedema, and wherein the absence of the mutation in the nucleic acid correlates with no increased risk of developing hereditary lymphedema. In some embodiments of this method, the at least one gene corresponds to the human Sox18 gene that encodes the amino acid sequence set forth in SEQ ID NO: 54.

In the methods of assaying for risk of developing hereditary lymphedema according to the invention, the assaying may identify the presence of the mutation, and the correlating step may identify the increased risk of the patient developing hereditary lymphedema.

A related method according to the invention is a method of screening a human subject for an increased risk of developing hereditary lymphedema comprising assaying nucleic acid of a human subject for a mutation that alters the encoded amino acid sequence of at least one polypeptide comprising an amino acid sequence of Table 3. In some embodiments of this method, the polypeptide comprises an amino acid sequence selected from the group consisting of SEQ ID NOS: 31-44, 46, 48, 50, 52, and 54, 207, 676, 859, and 861 in a manner that correlates with the risk of developing hereditary lymphedema, and it is expressly contemplated that the polypeptide may comprise the SOX18 amino acid sequence set forth in SEQ ID NO: 54.

A related aspect of the invention is drawn to methods of assaying or screening for risk of developing hereditary lymphedema as described above, wherein the method comprises at least one procedure selected from the group consisting of: (a) determining a nucleotide sequence of at least one codon of at least one polynucleotide of the human subject; (b) performing a hybridization assay to determine whether nucleic acid from the human subject has a nucleotide sequence identical to or different from one or more reference sequences; (c) performing a polynucleotide migration assay to determine whether nucleic acid from the human subject has a nucleotide sequence identical to or different from one or more reference sequences; and (d) performing a restriction endonuclease digestion to determine whether nucleic acid from the human subject has a nucleotide sequence identical to or different from one or more reference sequences.

A related aspect of the invention provides methods of assaying or screening for risk of developing hereditary lymphedema as described above, wherein the method comprises: performing a polymerase chain reaction (PCR) to amplify nucleic acid comprising the coding sequence of the LEC polynucleotide, and determining nucleotide sequence of the amplified nucleic acid.

Further provided by the invention is a method of screening for a hereditary lymphedema genotype in a human subject, comprising: (a) providing a biological sample comprising nucleic acid from said subject, and (b) analyzing the nucleic acid for the presence of a mutation altering the encoded amino acid sequence of the at least one allele of at least one gene in the human subject relative to a human gene encoding an amino acid sequence selected from the group consisting of SEQ ID NOs: 31-44, 46, 48, 50, 52, 54, 207, 676, 859, and 861, wherein the presence of a mutation altering the encoded amino acid sequence in the human subject in a manner that correlates with lymphedema in human subjects identifies a hereditary lymphedema genotype. In some embodiments of this method, the biological sample is a cell sample. In other embodiments of this method, the analyzing comprises sequencing a portion of the nucleic acid. In still further embodiments of this method, the human subject has a hereditary lymphedema genotype identified by the method of screening.

Another aspect of the invention provides a method of inhibiting lymphangiogenesis comprising administering to a subject an inhibitor of a LEC transmembrane polypeptide, wherein the LEC transmembrane polypeptide comprises an amino acid sequence selected from the group consisting of SEQ ID NOs: 31-34, 46 48, 207, 676, 859, and 861, and wherein the inhibitor is selected from the group consisting of (a) a soluble extracellular domain fragment of the LEC transmembrane polypeptide; (b) an antibody that binds to the extracellular domain of the LEC transmembrane polypeptide; (c) a polypeptide comprising an antigen binding domain of the antibody according to (b); and (d) an antisense nucleic acid complementary to the nucleic acid encoding the LEC transmembrane polypeptide or its complement. In some embodiments of the method, the inhibitor is a polypeptide comprising an extracellular domain fragment of an LEC polypeptide, wherein the sequence of the extracellular domain is selected from the group consisting of amino acids 1-152 of SEQ ID NO:31, amino acids 1-695 of SEQ ID NO:32 and amino acids 1-248 of SEQ ID NO:33. In some embodiments of the method, the subject is a human containing a tumor.

In a related aspect, the invention provides a method for modulating lymphangiogenesis in a mammalian subject comprising: administering to a mammalian subject in need of modulation of lymphangiogenesis an antisense molecule to a LEC polynucleotide, in an amount effective to inhibit transcription or translation of the polypeptide encoded by the LEC polynucleotide, wherein the LEC polynucleotide comprises a nucleotide sequence selected from the group consisting of SEQ ID NOS: 14-30, 45, 47, 49, AND 51, 208, 677, 860, and 862.

The methods of the invention also include methods for the treatment of disorders related to lymphatic endothelial cells as recited above which may involve the administration of such compounds to individuals exhibiting symptoms or tendencies related to such disorders.

In another aspect, the invention provides a method of treating hereditary lymphedema, comprising: (a) identifying a human subject with hereditary lymphedema and with a mutation that alters the encoded amino acid sequence of at least one polypeptide of the human subject, relative to the amino acid sequence of a polypeptide comprising an amino acid sequence selected from the group consisting of SEQ ID NOS: 31-44, 46, 48, 50, 52, 54, 207, 676, 859, and 861; and (b) administering to the subject a lymphatic growth factor selected from the group consisting of a VEGF-C polynucleotide, a VEGF-C polypeptide, a VEGF-D polynucleotide, and a VEGF-D polypeptide.

The invention also provides a method of treating hereditary lymphedema comprising: identifying a human subject with lymphedema and with a mutation in at least one allele of a gene encoding a LEC protein identified in Table 3, wherein the mutation correlates with lymphedema in human subjects, and with the proviso that the LEC protein is not VEGFR-3; and administering to the subject a composition comprising a lymphatic growth agent selected from the group consisting of VEGF-C polypeptides, VEGF-D polypeptides, VEGF-C polynucleotides, and VEGF-D polynucleotides. The invention also comprehends use of a lymphatic growth agent selected from the group consisting of VEGF-C polypeptides, VEGF-D polypeptides, VEGF-C polynucleotides, and VEGF-D polynucleotides in the manufacture of a medicament for the treatment of hereditary lymphedema resulting from a mutation in a LEC gene identified in Table 3, with the proviso that the gene is not VEGFR-3.

In addition, the invention encompasses methods for treating such diseases or disorders by administering compounds and other substances that modulate the overall activity of the target gene products. Compounds and other substances can effect such modulation either at the level of target gene expression or target protein activity. These treatment methods include the administration of a polypeptide or a polynucleotide according to the invention to an endothelial cell, e.g., a LEC and/or a BEC, or to an organism such as a human patient. An exemplary method according to this aspect of the invention is the administration of a therapeutic selected from the group consisting of an antisense polynucleotide capable of modulating the expression of at least one polynucleotide according to the invention, a polypeptide according to the invention, a polynucleotide according to the invention, an antibody or antibody fragment specifically recognizing a polypeptide according to the invention, a VEGF-C polynucleotide, a VEGF-C polypeptide, a VEGF-D polynucleotide, a VEGF-D polypeptide and a soluble VEGFR-3 polypeptide.

In another aspect, the invention provides a method of screening for an endothelial cell disorder or predisposition to the disorder, comprising obtaining a biological sample containing endothelial cell mRNA from a human subject; and measuring expression of a BEC or LEC gene from the amount of mRNA in the sample transcribed from the gene, wherein the BEC or LEC gene encodes a polypeptide identified in Table 3 or 4.

The invention relates to a method of inhibiting the growth of a lymphatic endothelial cell, the method comprising contacting the cell with a composition comprising at least one antibody conjugated to an agent capable of inhibiting the growth, wherein the agent is selected from the group consisting of a cytotoxic agent and a cytostatic agent, and wherein the antibody specifically binds to a polypeptide encoded by a polynucleotide comprising a sequence selected from the group consisting of SEQ ID NOS:14-17, 45, 47, 860 and 862. In specific embodiments of this method, the polypeptide comprises an amino acid sequence selected from the group consisting of SEQ ED NOS:31-34, 46, 48, 859 and 861.

The invention further relates to methods of detecting a lymphatic endothelial cell, the method comprising contacting the cell with a composition comprising at least one antibody conjugated to a detectable agent, such as a fluorescent molecule or a radiolabeled molecule. In specific embodiments, the antibody specifically binds to a polypeptide encoded by a polynucleotide comprising a sequence selected from the group consisting of SEQ ID NOS: 14-17, 45, 47, 860 and 862. In further specific embodiments of this method, the polypeptide comprises an amino acid sequence selected from the group consisting of SEQ ID NOS: 31-34, 46, 48, 859 and 861.

The invention still further relates to methods of isolating a lymphatic endothelial cell, comprising contacting the cell with a solid matrix comprising at least one antibody capable of binding to a transmembrane protein in the cell membrane of the cell, and isolating cells specifically bound to the antibody matrix. In specific embodiments, the antibody specifically binds to a polypeptide encoded by a polynucleotide comprising a sequence selected from the group consisting of SEQ ID NOS:14-17, 45, 47, 860 and 862. In further specific embodiments of this method, the polypeptide comprises an amino acid sequence selected from the group consisting of SEQ ID NOS:31-34, 46, 48, 859 and 861.

The invention also relates to the administration of an agonist or antagonist to a lymphatic endothelial cell, comprising selecting an antibody, a peptide or a small molecular weight compound that is capable of specifically binding to a lymphatic endothelial cell-specific protein, wherein the antibody, peptide or small molecular weight compound is an agonist or antagonist for a growth factor receptor, a cytokine receptor, a chemokine receptor, or a hemopoietic receptor, and contacting the antibody, peptide or small molecular weight compound with the lymphatic endothelial cell in need of growth stimulation or inhibition. In specific embodiments, such lymphatic endothelial cells are involved in lymphedema, lymphangioma, lymphangiomyeloma, lymphangiomatosis, lymphangiectasis, lymphosarcoma, and lymphangioscierosis.

The invention also relates to the administration of a cytotoxic or cytostatic drug to a lymphatic endothelial cell, comprising selecting an antibody, a peptide or a small molecular weight compound that is capable of specifically binding to a lymphatic endothelial cell-specific protein, wherein the antibody, peptide or small molecular weight compound is complexed to the cytotoxic or cytostatic drug. In specific embodiments, administration of such complexes is useful in the treatment of malignant tumor diseases prone to metastatic spread through the lymphatic system.

The invention also provides a method of monitoring the efficacy or toxicity of a drug on endothelial cells, comprising steps of measuring expression of at least one BEC or LEC gene in endothelial cells of a mammalian subject before and after administering a drug to the subject, wherein the at least one BEC or LEC gene encodes a polypeptide set forth in Table 3 or Table 4, and wherein changes in expression of the BEC or LEC gene correlates with efficacy or toxicity of the drug on endothelial cells.

The invention relates to a lymphatic endothelial cell marker protein comprising a polypeptide encoded by a polynucleotide selected from the group consisting of SEQ ID NOS:14-17; and a polynucleotide hybridizable under stringent conditions with any one of SEQ ID NOS:14-17. In specific embodiments, the lymphatic endothelial cell marker protein comprises a polypeptide selected from the group consisting of SEQ ID NOS:3 1-34.

The invention also relates to an antibody capable of specifically binding to a lymphatic endothelial cell marker protein comprising a polypeptide selected from the group consisting of SEQ ID NOS:31-34.

The invention further relates to a method of detecting a lymphatic endothelial cell, comprising contacting said cell with the antibody wherein said antibody is detectably labeled.

The invention still further relates to a method of inhibiting at least one biological activity of a lymphatic endothelial cell, comprising contacting the cell with an agent capable of binding to at least one polypeptide encoded by any one of SEQ ID NOS:14-17, 45, 47, 860 and 862, wherein the activity of the polypeptide is reduced relative to the activity of a polypeptide that is not contacted with the agent.

The invention also relates to a method of inhibiting the growth of a lymphatic endothelial cell, the method comprising contacting the cell with an antisense oligonucleotide capable of specifically binding to at least one polynucleotide selected from the group consisting of SEQ ID NOS:1-30, 45, 47, 860 and 862. In a specific embodiment, the antisense oligonucleotide consists essentially of about 12 to about 25 contiguous nucleotides of any one of SEQ ID NOS: 1-30, 45, 47, 860 and 862.

Additional features and variations of the invention will be apparent to those skilled in the art from the entirety of this application, and all such features are intended as aspects of the invention.

### BRIEF DESCRIPTION OF THE SEVERAL VIEWS OF THE DRAWINGS

FIG. 1: Examples of differentially expressed genes in LECs and BECs. Northern blotting and hybridization for the indicated transcripts. Equal loading was verified by probing with GAPDH. For the microarray analyses, RNA was extracted from LECs which were cultured in the presence of VEGF-C (LEC/+C). When validating the array results, RNA was extracted as a control also from cultures of LECs in which VEGF-C was not added (LEC/−C).

FIG. 2: Cytoskeletal structures, cadherin complexes and integrin α9 expression in BECs and LECs. Mixed cultures of LEC and BEC were double-stained for N-cadherin (a), VE-cadherin (c), β-catenin (e), plakoglobin (g), F-actin (i) and integrin α9 (k), and for the LEC-specific marker podoplanin (green; b, d, f, h, j, l). Expression of integrin α9 in the lymphatic (arrow) but not in blood vessel endothelia (arrowhead). Adjacent sections of human skin were stained with antibodies against integrin α9 (m), VEGFR-3 (n) or blood vessel endothelial antigen PAL-E (o).

### DETAILED DESCRIPTION OF THE INVENTION

A major role of the lymphatic vasculature is to remove an excess of the protein-rich interstitial fluid that continuously escapes from the blood capillaries, and to return it to the blood circulation (Witte, M. H., et al., Microsc. Res. Tech. 55:122-145. 2001; Karpanen, T., et al., J. Exp. Med. 194:F37-F42. 2001; Karkkainen, M. J., et al., Trends Mol. Med. 7:18-22. 2001). In addition, the lymphatic system provides constant immune surveillance by filtering lymph and its antigens through the chain of lymph nodes, and also serves as one of the major routes for absorption of lipids from the gut. It has been known for a long time-that in many types of cancer the lymphatic vessels provide a major pathway for tumor metastasis, and regional lymph node dissemination correlates with the progression of the disease. Hereditary lymphedema, post-surgical secondary lymphedema and lymphatic obstruction in filariasis, are all characterized by disabling and disfiguring swelling of the affected areas, linked to the insufficiency or obstruction of the lymphatics. Witte, M. J., et al., Microsc. Res. Tech 55:122-145 (2001).

In spite of the importance of lymphatic vessels in medicine, the cell biology of this part of the vascular system has received little attention until recently. Studies during the past four years have uncovered the existence of the lymphatic specific vascular endothelial growth factors VEGF-C and VEGF-D, which serve as ligands for the receptor tyrosine kinase VEGFR-3, and demonstrated their importance for the normal development of the lymphatic vessels (See, Jeltsch, M., et al., Science 276:1423-1425 (1997); Veikkola, T., et al., EMBO J. 20:1223-1231 (2001); Mäkinen, T., et al., Nat. Med. 7:199-205 (2001)). These molecules also appear to be involved in the development of lymphedema and lymphatic metastasis (Karpanen, T., et al., J. Exp. Med. 194:F37-F42 (2001); Karkkainen, M. J., et al., Trends Mol. Med. 7:18-22. 2001).

The growth factor Vascular Endothelial Growth Factor C (VEGF-C), as well as native human, non-human mammalian, and avian polynucleotide sequences encoding VEGF-C, and VEGF-C variants and analogs, have been described in detail in International Patent Application Number PCT/US98/01973, filed Feb. 2, 1998 and published on Aug. 6, 1998 as International Publication Number WO 98/33917; in Joukov et al., J. Biol. Chem., 273(12): 6599-6602 (1998); and in Joukov et al., EMBO J., 16(13): 3898-3911 (1997), all of which are incorporated herein by reference in their entirety. As explained therein in detail, human VEGF-C (SEQ ID NO: 863) is initially produced in human cells as a prepro-VEGF-C polypeptide of 419 amino acids. A cDNA encoding human VEGF-C (SEQ ID NO: 864) has been deposited with the American Type Culture Collection (ATCC), 10801 University Blvd., Manassas, Va. 20110-2209 (USA), pursuant to the provisions of the Budapest Treaty (Deposit date of 24 Jul. 1995 and ATCC Accession Number 97231). VEGF-C sequences from other species also have been reported. See Genbank Accession Nos. MMU73620 (Mus musculus); and CCY15837 (Coturnix coturnix) for example, incorporated herein by reference.

The prepro-VEGF-C polypeptide is processed in multiple stages to produce a mature and most active VEGF-C polypeptide of about 21-23 kD, as assessed by SDS-PAGE under reducing conditions (SEQ ID NO: 863). Such processing includes cleavage of a signal peptide (residues 1-31); cleavage of a carboxyl-terminal peptide (corresponding approximately to amino acids 228-419 and having a pattern of spaced Cysteine residues reminiscent of a Balbiani ring 3 protein (BR3P) sequence [Dignam et al., Gene, 88:133-40 (1990); Paulsson et al., J. Mol. Biol., 211:331-49 (1990)]) to produce a partially-processed form of about 29 kD; and cleavage (apparently extracellularly) of an amino-terminal peptide (corresponding approximately to amino acids 32-103) to produced a fully-processed mature form of about 21-23 kD. Experimental evidence demonstrates that partially-processed forms of VEGF-C (e.g., the 29 kD form) are able to bind VEGFR-3 (Flt4 receptor), whereas high affinity binding to VEGFR-2 occurs only with the fully processed forms of VEGF-C. It appears that VEGF-C polypeptides naturally associate as non-disulfide linked dimers.

It has been demonstrated that amino acids 103-227 of VEGF-C are not all critical for maintaining VEGF-C functions. A polypeptide consisting of amino acids 113-213 (and lacking residues 103-112 and 214-227) retains the ability to bind and stimulate VEGF-C receptors, and it is expected that a polypeptide spanning from about residue 131 to about residue 211 will retain VEGF-C biological activity. The Cysteine residue at position 156 has been shown to be important for VEGFR-2 binding ability. However, VEGF-C ΔC₁₅₆ polypeptides (i.e., analogs that lack this Cysteine due to deletion or substitution) remain potent activators of VEGFR-3. The Cysteine at position 165 of VEGF-C polypeptide is essential for binding either receptor, whereas analogs lacking the Cysteine at positions 83 or 137 compete with native VEGF-C for binding with both receptors and stimulate both receptors.

VEGF-D is structurally and functionally most closely related to VEGF-C [see U.S. Pat. No. 6,235,713 and International Patent Publ. No. WO 98/07832, incorporated herein by reference]. See SEQ ID NO: 866 for the polynucleotide sequence of VEGF-D; the encoded amino acid sequence is set forth in SEQ ID NO: 865. Like VEGF-C, VEGF-D is initially expressed as a prepro-peptide that undergoes N-terminal and C-terminal proteolytic processing, and forms non-covalently linked dimers. VEGF-D stimulates mitogenic responses in endothelial cells in vitro. During embryogenesis, VEGF-D is expressed in a complex temporal and spatial pattern, and its expression persists in the heart, lung, and skeletal muscles in adults. Isolation of a biologically active fragment of VEGF-D designated VEGF-DΔNΔC, is described in International Patent Publication No. WO 98/07832, incorporated herein by reference. VEGF-DΔNΔC consists of amino acid residues 93 to 201 of VEGF-D (SEQ ID NO: 26) optionally linked to the affinity tag peptide FLAG®, or other sequences.

The prepro-VEGF-D polypeptide has a putative signal peptide of 21 amino acids and is apparently proteolytically processed in a manner analogous to the processing of prepro-VEGF-C. A “recombinantly matured” VEGF-D lacking residues 1-92 and 202-354 retains the ability to activate receptors VEGFR-2 and VEGFR-3, and appears to associate as non-covalently linked dimers. Thus, preferred VEGF-D polynucleotides include those polynucleotides that comprise a nucleotide sequence encoding amino acids 93-201. The guidance provided above for introducing function-preserving modifications into VEGF-C polypeptides is also suitable for introducing function-preserving modifications into VEGF-D polypeptides. As another aspect of the invention, practice of the invention methods is contemplated wherein VEGF-D polypeptides are employed in lieu of VEGF-C polypeptides.

When compared with the blood vascular endothelium, the lymphatic endothelium exhibits specific morphological and molecular characteristics. For example, the lymphatic capillaries are larger than blood capillaries, they have an irregular or collapsed lumen with no red blood cells, a discontinuous basal lamina, overlapping intercellular junctional complexes and anchoring filaments that connect the lymphatic endothelial cells to the extracellular matrix (Witte, M. H., et al., Microsc. Res. Tech. 55:122-145 (2001)). Unlike the blood capillaries, the lymphatic capillaries lack pericyte coverage. At the molecular level several lymphatic specific markers have been identified, including VEGFR-3, the Prox-1 transcription factor, the hyaluronan receptor LYVE-1, the membrane mucoprotein podoplanin, the beta-chemokine receptor D6, the cytoskeletal proteins desmoplakin I and II and the macrophage mannose receptor I (Wigle, J. T. & Oliver, G, Cell 98:769-778 (1999); Banedji, S., et al., J. Cell Biol. 144:789-801 (1999): Breiteneder-Geleff, S., et al., Am. J. Pathol. 154:385-394 (1999): Nibbs, R. J., et al., Am. J. Pathol. 158:867-877 (2001); Ebata, N., et al., Microvasc. Res. 61:40-48. (2001); Irjala, H., et al., J. Exp. Med. 194:1033-1041 (2001)). The present invention relates to the genetic identity of lymphatic capillary endothelial cells versus blood vascular endothelial cells using a gene profiling approach.

“Stringent hybridization conditions” or “stringent conditions” refer to conditions under which a nucleic acid such as an oligonucleotide will specifically hybridize to its target sequence. Stringent conditions are sequence-dependent and will be different in different circumstances. Longer nucleic acids hybridize specifically at higher temperatures than shorter sequences. Generally, stringent conditions are selected to be about 5° C. lower than the thermal melting point (Tₘ) for the specific sequence at a defined ionic strength and pH. The Tₘ is the temperature (under defined ionic strength, pH and nucleic acid concentration conditions) at which 50% of the nucleic acids complementary to the target sequence hybridize to the target sequence at equilibrium. The term “complementary” refers to standard Watson-Crick base pairing between nucleotides of two nucleic acid molecules. Typically, stringent conditions will be those in which the salt concentration is less than about 1.0 M sodium ion, typically about 0.01 to 1.0 M sodium ion (or other salts) at pH 7.0 to 8.3 and at a temperature that is at least about 30° C. for short probes, primers or oligonucleotides (e.g., 10. to 50 nucleotides) and at least about 60° C. for longer probes, primers or oligonucleotides. Stringent conditions also can be achieved with the addition of destabilizing agents, such as formamide, as is known in the art Exemplary stringent hybridization conditions are hybridization at 42° C. for 20 hours in a solution containing 50% forrnamide, 5×SSPE, 5× Denhardt's solution, 0.1% SDS and 0.1 mg/ml denatured salmon sperm DNA, with a wash in 1×SSC, 0.1% SDS for 30 minutes at 65° C.

According to the invention, distinct gene expression profiles for blood vascular and lymphatic endothelial cells have been discovered. These results provide new insights into the phenotypic diversity of endothelial cells and reveal new potential lymphatic endothelial molecules, some of which could provide important targets for the therapy of diseases characterized by abnormal angiogenesis or lymphangiogenesis.

Differences in the expression of genes encoding proteins involved in inflammatory processes were found, as well as in those mediating cell-cell and cell-matrix interactions. Furthermore, several previously unknown genes were identified in the context of endothelial cell biology, which were differentially expressed in the two cell lineages. Several of the genes were originally cloned from neural tissues, including genes involved in the uptake of synaptic macromolecules and in synapse formation and remodeling (neuronal pentraxins I and II (Kirkpatrick, L. L., et al., J. Biol. Chem.. 275:17786-17792. 2000), in the trafficking of synaptic vesicles (NAP-22 (Yamamoto, Y., et al., Neurosci. Lett. 224:127-130. 1997), piccolo (Fenster, S. D., et al., Neuron 25:203-214 (2000)) and in the axon growth and guidance (Nr-CAM (Grumet, M., Cell Tissue Res. 290:423-428 (1997), reelin (Rice, D. S. & Curran, T., Annu. Rev. Neurosci. 24:1005-1039 (2001)).

In addition, the LECs especially expressed a number of as yet uncharacterized genes, which were originally cloned and highly expressed in nervous tissues (KIAA genes (Kikuno, R., et al., Nucleic Acids Res. 30:166-168. 2002). The gene expression profiling data disclosed herein therefore support the view that the same molecular mechanisms that are involved in governing neural cell positioning, in guiding axonal growth cones to their specific targets and in synaptogenesis may also be commonly used in the development of the vascular system and in the establishment of BEC and LEC identity. Some other signaling molecules first described in the developing nervous system have already been implicated in the development of the vasculature and vice versa (Shima and Mailhos, Curr. Opin. Genet. Dev. 10:536-542 (2000); Oosthuyse, et al., Nat. Genet. 28:131-138 (2001); Sondell, et al., Eur. J. Neurosci. 12:4243-4254 (2000)).

In the LECs, expression of several genes previously shown to be expressed in smooth muscle cells (SMCs) and pericytes was observed, such as matrix Gla, a mineral binding extracellular matrix protein involved in the inhibition of vascular and tissue calcification (Luo, G, et al., Nature 386:78-81 (1997)), monoamine oxidase A, the main degradative enzyme of monoamine hormones and neurotransmitters (Rodriguez, M. J., et al., Cell Tissue Res. 304:215-220 (2001)), integrin α9 (Palmer, E. L., et al., J. Cell Biol. 123:1289-1297 (1993)) and apolipoprotein D (Hu, C. Y, et al., J. Neurocytol. 30:209-218 (2001)). Some similarity of gene expression patterns between LECs and SMCs could be related to the lack of SMC around lymphatic capillaries. Instead, LECs may carry out some SMC functions by themselves. For example, lymph flow is maintained due to the intrinsic contractility of the LECs (Witte, M. H., et al., Microsc. Res. Tech. 55:122-145 (2001)), reminiscent of the ability of vascular SMCs to contract.

Molecular discrimination of the lymphatic and blood vessels is essential in studies of diseases involving the blood and/or lymphatic vessels and in the targeted treatment of such diseases. To date, several lymphatic endothelial specific markers have been identified, but some of them are expressed only in a subset of the lymphatic vessels, while others also occur in some blood vessel endothelia or in other cell types and their expression patterns may change in pathological conditions (for example, VEGFR-3 (Valtola, R., et al., Am. J. Pathol. 154:1381-1390. 1999)). Identification of new vascular markers according to the invention should provide a more reliable analysis of the blood and lymphatic vessels in pathological situations and eventually better diagnosis and treatment. Furthermore, inhibition of the function of certain molecules involved in the regulation of angiogenesis and/or lymphangiogenesis is known to prevent tumor growth and metastasis, and stimulation of the growth of blood or lymphatic vessels has been shown to be beneficial in several pathological conditions. Thus the BEC and LEC specific molecular regulators identified according to the invention may provide new targets for the treatment of diseases characterized by abnormal angiogenesis and lymphangiogenesis.

Several of the new LEC genes encode transmembrane proteins that may be specific molecular markers for lymphatic endothelial cells (Table 6). These genes and encoded proteins are useful for targeted treatment of diseases that involve lymphatic vessels. They may also be useful for preparing antibodies, as antibodies against LEC-specific proteins can be used to discriminate between blood and lymphatic vessels in pathological and physiological situations. Antibodies may also be useful for the isolation of lymphatic endothelial cells. These proteins may also play a role in the regulation of lymphangiogenesis, and can provide new candidate genes for diseases that involve lymphatic vessels, such as lymphedema.

The lymphatic endothelial cell specific surface molecules can be used for molecular drug targeting with antibodies, peptides and small-molecular weight compounds, which can act as agonists or antagonists for growth factor receptor, cyto- and chemokine receptor, and hemopoietin receptor signaling, cell adhesion and cell interaction with extracellular matrix or with other cell surface molecules. Such molecules can also be used for targeting of cytotoxic or cytostatic drugs into the lymphatic endothelial cells and for the attachment of electron-dense, radio-opaque or radioactive markers for imaging of disease processes associated with the lymphatic vessels. Such diseases include lymphedema, lymphangioma, lymphangiomyoma, lymphangiomatosis, lymphangiectasis, lymphosarcoma and lymphangiosclerosis.

The lymphatic endothelial cell surface molecules may be used for targeting of gene therapy for example by antibody-coated liposomes (containing proteins or genes as cargo) or by viral transducing vectors such as adenoviruses, adeno-associated viruses or lentiviruses having modified capsid/envelope proteins. The manipulation of lymphatic endothelial cell specific molecules may be applicable to treatment of disease processes associated with tissue edema by increasing fluid transport across the lymphatic vessel wall for example by modulating endothelial cell-cell or cell-matrix interactions or via stimulating transendothelial transport. Targeting of the lymphatic endothelial cells for example with cytotoxic or cytostatic compounds is contemplated to be valuable in malignant tumor diseases prone to metastatic spread via the lymphatic system.

The lymphatic endothelial cell molecules may allow the improved in vitro growth of lymphatic endothelial cells as well as in vitro tissue engineering of lymphatic vessels for use in diseases where the lymphatics have been damaged, such as after surgery and in various forms of lymphedema. Ligands of the cell surface proteins may further be applied to coat various polymeric matrices for the adhesion of cells in, e.g., bioimplants.

The lymphatic endothelial-cell-specific molecules such as surface molecules can provide important tools for the modulation of inflammatory, autoimmune and infectious processes involving leukocyte migration and immune recognition as well as the stimulation of secondary immune responses. Such processes include the migration of antigen presenting cells into the lymphatic system including lymph nodes as well as fransendothelial cell trafficking of lymphocytes and other leukocyte subclasses and the homing, survival and function of the various classes of leukocytes.

These molecules may allow one to modulate the metabolism of fatty acids including fatty acid/chylomicron absorption from the gut and regulation of fat accumulation in adipose tissue in various organs such as in the subcutaneous tissue and in the arterial wall.

Lymphatic endothelial-cell-specific molecules may further allow one to modulate the metabolism of fatty acids including fatty acid/chylomicron absorption from the gut and regulation of fat accumulation in adipose tissue in various organs such as in the skin subcutaneous tissue and in the arterial wall.

The lymphatic-cell-specific transmembrane proteins are expected to function in cell adhesion (e.g., adhesion between lymphatic endothelial cell-lymphatic endothelial cell, lymphatic endothelial cell-smooth muscle cell, lymphatic endothelial cell-immune system cell such as lymphocyte or dendritic cell), cell-extracellular matrix contacts, or as receptors such as growth factor, cytokine, chemokine or microbial receptors or ion channels. The transmembrane proteins connect to intracellular molecules that can induce cell growth, cell migration, cell apoptosis, cell differentiation or cell adhesion or other cellular functions specific for endothelial cells such as expression of adhesion receptors for leukocytes, release of nitric oxide, antigoagulant proteins, uptake of fluid and proteins from surrounding tissues and fat from gut or adipose tissues. TM proteins with short intracellular domains can function as auxiliary receptors in complex with other TM proteins.

The transmembrane proteins and their intracellular binding partner molecules can be used as molecular markers for lymphatic endothelial cells in normal and disease conditions, and to discriminate between blood and lymphatic vessels in pathological and physiological situations.

Antibodies against lymphatic specific transmembrane proteins, as well as peptides and small molecular compounds binding to extracellular domains of lymphatic-specific TM proteins can be used for the attachment of electron-dense, radio-opaque or radioactive markers for imaging of disease processes associated with the lymphatic vessels. Such diseases include lymphedema, lymphangioma, lymphangiomyoma, lymphangiomatosis, lymphangiectasis, lymphosarcoma and lymphangiosclerosis. Similarly, the lymphatic vessels can be visualized, e.g., during therapy of patients suffering from insufficient lymphatic growth, such as in lymphedema, or alternatively during treatment aiming to prevent lymphatic growth, e.g., in tumors, thereby facilitating the monitoring of the therapeutic method of the invention.

Antibodies against LEC-specific TM proteins are also expected to be useful for the isolation of lymphatic endothelial cells.

Antibodies against lymphatic-specific transmembrane proteins, or peptides or small-molecule compounds binding to the extracellular domain of lymphatic-specific TM proteins are expected to be useful in targeting drug delivery to lymphatic endothelial cells, e.g., by coupling an antibody, peptide or small-molecule compound to a cytotoxic or cytostatic compound. Such coupled compounds are useful as therapeutics in the treatment of malignant tumor diseases prone to metastatic spread via the lymphatic system, as well as in ameliorating a symptom associated with any such disease. The antibodies, peptides or small-molecule compounds can also be coupled to stimulatory lymphatic endothelial molecules such as growth factors, cytokines and chemokines to promote stimulation.

Additionally, antibodies against lymphatic-specific TM proteins or peptides, or small-molecule compounds binding to the extracellular domain of lymphatic-specific TM proteins, may be used for targeting of gene therapy, for example, by antibody-coated liposomes (containing proteins, genes or other molecules as cargo) or by viral transducing vectors such as adenoviruses, adeno-associated viruses, lentiviruses, or the like, having modified capsid/envelope proteins. The manipulation of lymphatic endothelial-cell-specific molecules are expected to be applicable to the treatment of disease processes associated with tissue edema due to the relative absence, or relative dysfunction, of lymphatic vessels, which can result from an infection, surgery, radiotherapy or a genetic defect by increasing fluid transport across the lymphatic vessel wall, for example by modulating endothelial cell-cell or cell-matrix interactions or by stimulating transendothelial transport.

The lymphatic endothelial cell molecules are expected to improve the in vitro growth of lymphatic endothelial cells, as well as the in vitro tissue engineering of lymphatic vessels for use in treating disorders or diseases where the lymphatics have been damaged, such as after surgery, in various forms of lymphedema, and in other applications as described herein. Ligands of the cell-surface proteins may further be applied as a coating to various polymeric matrices for the adhesion of cells in, e.g., bioimplants.

Inflammatory, autoimmune and infectious processes involving leukocyte migration and immune recognition, such as migration of antigen-presenting cells into the lymphatic system, including lymph nodes, as well as transendothelial cell trafficking of lymphocytes and other leukocyte subclasses and the homing, survival and function of the various classes of leukocytes can be modulated by targeting endothelial-cell-specific TM proteins, which mediate these cell adhesion processes.

Upregulation of lymphatic-specific genes in, e.g., cancer are expected to be useful as diagnostic markers, and monitoring such upregulated expression with an antibody against a lymphatic endothelial-cell-specific protein, e.g., by immunostaining of tissue(s) or by using a probe hybridizable to a lymphatic endothelial-cell-specific mRNA, e.g., under stringent hybridization conditions as described herein, is contemplated.

Lymphatic endothelial-cell-specific transcription factors are expected to be useful for the differentiation of lymphatic endothelial cells from embryonic stem cells, endothelial precursor cells, or blood vascular endothelial cells.

The lymphatic endothelial transcription factors are expected to improve the in vitro growth of lymphatic endothelial cells, as well as to facilitate in vitro tissue engineering of lymphatic vessels for use in treating disorders or diseases where the lymphatics have been damaged, such as after surgery, in various forms of lymphedemna, and in other applications disclosed herein.

Intracellular signaling proteins participating in signaling pathways regulating lymphatic endothelial cell proliferation, differentiation, apoptosis, migration or adhesion are expected to be useful targets for small-molecule compounds inhibiting these signaling events, and cellular functions dependent on such signaling. Signaling proteins are also expected to participate in VEGFR-3 signaling pathways, and will be useful in modulating cellular activities controlled, at least in part, by VEGFR-3 signaling, such as lymphangiogenesis.

The lymphatic endothelial cell molecules are expected to improve the in vitro growth of lymphatic endothelial cells as well as in vitro tissue engineering of lymphatic vessels for use in treating diseases or disorders where the lymphatics have been damaged, such as after surgery, in various forms of lymphedema, and in other applications as described herein.

Lymphatic-specific transcription factors are also expected to be useful in modulating gene expression in endothelial cells to induce the expression of other lymphatic-specific genes in, for example, blood vascular endothelial cells or endothelial precursor cells.

Lymphatic-specific gene transcripts are expected to provide useful targets for RNA interference (RNAi)-induced inhibition of expression. RNAi technology is expected to be useful in the methods according to the invention, such as therapeutic methods effective in treating hyper- and hypo-proliferative endothelial-cell-associated diseases and disorders, as well as methods of ameliorating a symptom of any such disease or disorder. RNAi methodologies are known in the art and known RNAi technologies are contemplated as useful in various aspects of the invention. See Fire et al., Nature 391:806-811. (1998) and Sharp, P., Genes and Dev. 13:139-141. (1999), each of which is incorporated herein by reference. It is preferred that RNAi compounds be double-stranded RNA molecules corresponding to part or all of a coding region of a desired target for expression.

As noted, several of the new LEC genes encode transcription factors, which may control cellular fate (iroquois-related homeobox gene), and may have an important role in the differentiation of lymphatic endothelial cells. Transcription factors disclosed herein may control transcription of genes involved for example in the proliferation of lymphatic endothelial cells, and may be important molecular regulators of lymphatic growth (Table 5). Lymphatic endothelial cell specific transcription factors can be used for the differentiation of lymphatic endothelial cells from embryonic stem cells, endothelial precursor cells or from blood vascular endothelial cells.

The lymphatic endothelial transcription factors may allow the improved in vitro growth of lymphatic endothelial cells as well as in vitro tissue engineering of lymphatic vessels for use in diseases where the lymphatics have been damaged, such as after surgery and in various forms of lymphedema.

Polynucleotides of the Invention

In general, the isolated polynucleotides of the invention include the LEC and BEC polynucleotides exhibiting differential expression and identified in Tables 3, 4, 14, 15 and 16. The sequences of these polynucleoides are provided in Table 16, associated with their known database accession numbers, where applicable. In Tables 14 and 15, these accession numbers are correlated with unique sequence identifiers, thus permitting identification by sequence identifier of each citation to an accession number. The polynuleotide sequences may include a coding region and may include non-coding flanking sequences, which are readily identifiable by one of skill in the art. The invention contemplates polynucleotides comprising part, or all, of a coding region, with or without flanking regions, e.g., poly A sequences, 5′ non-coding sequences, and the like. The polynucleotides of the present invention also include, but are not limited to, a polynucleotide that hybridizes to the complement of the nucleotide sequence of any one of SEQ ID NOS: 1-30, 45, 47, 49 and 51 under highly stringent hybridization conditions; a polynucleotide that hybridizes to the complement of the nucleotide sequence of any one of SEQ ID NOS: 1-30, 45, 47, 49 and 51 under moderately stringent hybridization conditions; a polynucleotide which is an allelic variant of any polynucleotide recited above; a polynucleotide which encodes a species homologue of any of the proteins recited above; or a polynucleotide that encodes a polypeptide comprising a specific domain or truncation of the polypeptide of any one of SEQ ID NOS: 31-44, 46, 48, 50 and 52. Such polynucleotides hybridize under the above conditions to the complement of any one of SEQ ID NOS: 1-30, 45, 47,49 and 51 or to a fragment of any one of SEQ ID NOS: 1-30, 45, 47, 49 and 51 wherein the fragment is greater than at least about 10 bp, and, in alternate embodiments, is about 20 to about 50 bp, or is greater than about 100 bp, 200 bp, 300 bp, 400 bp, 500 bp, 600 bp, 700 bp, or 800 bp, where appropriate.

The polynucleotides of the invention also provide polynucleotides that are variants of the polynucleotides recited above. Typically, such a variant sequence varies from one of those listed herein by no more than about 20%, ie., the number of individual nucleotide substitutions, additions, and/or deletions in a similar sequence, as compared to the corresponding reference sequence, divided by the total number of nucleotides in the variant sequence is about 0.2 or less. Such a sequence is said to have 80% sequence identity to the listed sequence. Such a variant sequence can be routinely identified by applying the foregoing algorithm.

In one embodiment, a variant polynucleotide sequence of the invention varies from a listed sequence by no more than 10%, i.e., the number of individual nucleotide substitutions, additions, and/or deletions in a variant sequence, as compared to the corresponding reference sequence, divided by the total number of nucleotides in the variant sequence is about 0.1 or less. Such a sequence is said to have 90% sequence identity to the listed sequence. Such a variant sequence can be routinely identified by applying the foregoing algorithm.

In an alternate embodiment a variant sequence of the invention varies from a listed sequence by no more than by no more than 5%, i.e., the number of individual nucleotide substitutions, additions, and/or deletions in a variant sequence, as compared to the corresponding reference sequence, divided by the total number of nucleotides in the variant sequence is about 0.05 or less. Such a sequence is said to have 95% sequence identity to the listed sequence. Such a variant sequence can be routinely identified by applying the foregoing algorithm.

In yet another alternate embodiment, a variant sequence of the invention varies from a listed sequence by no more than 2%, ie., the number of individual nucleotide substitutions, additions, and/or deletions in a variant sequence, as compared to the corresponding reference sequence, divided by the total number of nucleotides in the variant sequence is about 0.02 or less. Such a sequence is said to have 98% sequence identity to the listed sequence. Such a variant sequence can be routinely identified.

A polynucleotide according to the invention can be joined to any of a variety of other nucleotide sequences by well-established recombinant DNA techniques (see Sambrook J et al. (2d Ed.; 1989) Molecular Cloning: A Laboratory Manual, Cold Spring Harbor Laboratory, Cold Spring Harbor, N.Y.). Useful nucleotide sequences for joining to polypeptides include an assortment of vectors, e.g., plasmids, cosmids, lambda phage derivatives, phagemids, and the like, that are well known in the art. Accordingly, the invention also provides a vector including a polynucleotide of the invention and a host cell containing the polynucleotide. In general, the vector contains an origin of replication functional in at least one organism, convenient restriction endonuclease sites, and a selectable marker for the host cell. Vectors according to the invention include expression vectors, replication vectors, probe generation vectors, sequencing vectors, and retroviral vectors. A host cell according to the invention can be a prokaryotic or eukaryotic cell and can be a unicellular organism or part of a multicellular organism. Large numbers of suitable vectors and promoters are known to those of skill in the art and are commercially available for generating the recombinant constructs of the present invention.

The sequences falling within the scope of the present invention are not limited to the specific sequences herein described, but also include allelic variations thereof. Allelic variations can be routinely determined by comparing the sequence provided in any one of SEQ ID NOS: 1-30, 45, 47, 49 and 51, a representative intermediate fragment thereof, or a nucleotide sequence at least 99.9% identical to any one of SEQ ID NOS: 1-30, 45, 47, 49 and 51 with a sequence from another isolate of the same species. Furthermore, to accommodate codon variability, the invention includes nucleic acid molecules coding for the same amino acid sequences as do the specific open reading frames (ORFs) disclosed herein. In other words, in the coding region of an ORF, substitution of one codon for another which encodes the same amino acid is expressly contemplated.

Unless provided for otherwise here, all terms are defined as is known in the art, for example as employed in U.S. Pat. No. 6,350,447, incorporated herein by reference.

Also contemplated are antisense polynucleotides based on the sequence of any of the LEC or BEC polynucleotides according to the invention. Such antisense polynucleotides are substantially complementary (e.g., at least 90% complementarity), and preferably perfectly complementary, to sequences of the polynucleotides of the invention, or fragments thereof, set out in the sequence listing, Tables 3, 4, 14-16, and throughout this disclosure that are differentially expressed in LECs and BECs. These polynucleotide sequences include any of SEQ ID NOS: 1-30, 45, 47, 49 and 51, or a fragment thereof comprising at least 10 contiguous nucleotides. Antisense nucleic acid comprises a nucleotide sequence that is complementary to a “sense” nucleic acid encoding a protein (e.g., complementary to the coding strand of a double-stranded cDNA molecule or complementary to an mRNA sequence). Methods for designing and optimizing antisense nucleotides are described in Lima et al., (J Biol Chem,; 272:626-38. 1997) and Kurreck et al., (Nucleic Acids Res.,; 30:1911-8. 2002). In one aspect, antisense nucleic acid molecules are provided that comprise a sequence complementary to at least about 10, 25, 50, 100, 250 or 500 nucleotides or an entire coding strand. An antisense nucleic acid of the invention can be constructed using chemical synthesis or enzymatic ligation reactions using procedures known in the art.

In one embodiment, an antisense nucleic acid molecule is antisense to a “coding region” of the coding strand of a nucleotide sequence encoding a polypeptide of the invention. The term “coding region” refers to the region of the nucleotide sequence comprising codons which are translated into amino acid residues. In another embodiment, the antisense nucleic acid molecule is antisense to a “conceding region” of the coding strand of a nucleotide sequence encoding the polynucleotide. The term “conceding region” refers to 5′ and 3′ sequences which flank the coding region that are not translated into amino acids (i.e., also referred to as 5′ and 3′ untranslated regions).

Antisense nucleic acids of the invention can be designed according to the rules of Watson and Crick or Hoogsteen base pairing. The antisense nucleic acid molecule can be complementary to the entire coding region of the mRNA of the polynucleotide of the invention, but more preferably is an oligonucleotide that is antisense to only a portion of the coding or noncoding region of the mRNA. An antisense oligonucleotide can be, for example, about 5, 10, 15, 20, 25, 30, 35, 40, 45, or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

The antisense nucleic acid molecules of the invention are typically administered to a subject or generated in situ such that they hybridize or bind to cellular mRNA and/or genomic DNA encoding the complementary polynucleotide, thereby inhibiting expression of the protein (e.g., by inhibiting transcription and/or translation). The hybridization can reflect conventional nucleotide complementarity to form a stable duplex, or, for example, in the case of an antisense nucleic acid molecule that binds to DNA duplexes, through specific interactions in the major groove of the double helix.

An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). Additional routes of antisense therapy may be used in the invention, e.g., topical administration, transdermal administration [reviewed by Brand in Curr Opin. Mol. Ther. 3.244-8. 2001] antisense administration using nanoparticulate systems [Lambert et al., Adv. Drug. Deliv. Rev. 47:99-112. 2001], or administration of antisense nucleotides conjugated with peptide [Juliano et al., Curr. Opin. Mol. Ther. 2:297-303. 2000].

The invention further contemplates use of the polynucleotides of the invention for gene therapy or in recombinant expression vectors which produce polynucleotides or polypeptides of the invention that can regulate an activity of LEC genes, and are useful in therapy of LEC disorders such as lymphedema. Delivery of a functional gene encoding a polypeptide of the invention to appropriate cells is effected ex vivo, in situ, or in vivo by use of vectors, including viral vectors (e.g., adenovirus, adeno-associated virus, or a retrovirus), or ex vivo by use of physical DNA transfer methods (e.g., liposomes or chemical treatments). See, for example,. Anderson, Nature, supplement to vol. 392, no. 6679, pp. 25-20 (1998). For additional reviews of gene therapy technology see Friedmann, (Science, 244: 1275-1281. 1989); Verma, (Scientific American: 263:68-72, 81-84. 1990); and Miller, (Nature, 357: 455-460. 1992). Introduction of any one of the nucleotides of the present invention or a gene encoding a polypeptide of the invention can also be accomplished with extrachromosomal substrates (transient expression) or artificial chromosomes (stable expression). Cells may also be cultured ex vivo in the presence of proteins of the present invention in order to proliferate or to produce a desired effect on, or activity in, such cells. In another embodiment, cells comprising vectors expressing the polynucleotides or polypeptides of the invention may be cultured ex vivo and administered to an individual in need of treatment for an LEC disease or disorder.

Given the foregoing disclosure of the nucleic acid constructs, it is possible to produce the gene product of any of the genes comprising the sequence of any of SEQ ID NOS: 1-30, 45, 47, 49 and 51 by routine recombinant DNA/RNA techniques. A variety of expression vector/host systems may be utilized to contain and express the coding sequence. These include, but are not limited to, microorganisms such as bacteria transformed with recombinant bacteriophage, plasmid, phagemid, or cosmid DNA expression vectors; yeast transformed with yeast expression vectors; insect cell systems infected with viral expression vectors (e.g., baculovirus); plant cell systems transfected with virus expression vectors (e.g., Cauliflower Mosaic Virus, CaMV; Tobacco Mosaic Virus, TMV) or transformed with bacterial expression vectors (e.g., Ti or pBR322 plasmid); or even animal cell systems. Mammalian cells that are useful in recombinant protein productions include, but are not limited to, VERO cells, HeLa cells, Chinese hamster ovary (CHO) cells, COS cells (such as COS-7), WI38, BHK, HepG2, 3T3, RIN, MDCK, A549, PC12, K562 and HEK 293 cells.

Polypeptides of the Invention

In general, the isolated LEC and BEC polypeptides of the invention are encoded by the above-described differentially expressed LEC and BEC polynuleotides of the invention. The sequences of the LEC and BEC polypeptides are provided in Table 16, associated with their known database accession numbers, where applicable. In Tables 14 and 15, these accession numbers are correlated with unique sequence identifiers, thus permitting identification by sequence idenfier of 4each citation to an accession number. The isolated polypeptides of the invention include, but are not limited to, a polypeptide comprising: the amino acid sequences set forth as any one of SEQ ID NOS.: 31-44, 46, 48, 50 and 52 or an amino acid sequence encoded by any one of the nucleotide sequences set forth in SEQ ID NOS.: 1-30, 45, 47, 49 and 51, or the corresponding full length or mature protein. The invention also provides biologically active or immunologically active variants of any of the amino acid sequences set forth as SEQ ID NOS.: 31-44, 46, 48, 50 and 52, or the corresponding full length or mature protein suitable variant polypeptides have sequences that are at least about 65%, at least about 70%, at least about 75%, at least about 80%, at least about 85%, 86%, 87%, 88%, 89%, at least about 90%, 91%, 92%, 93%, 94%, typically at least about 95%, 96%, 97%, more typically at least about 98%, or most typically at least about 99% amino acid identity, that retain biological activity. Fragments of the proteins of the present invention which comprise at least 10 contiguous amino acids of a sequence disclosed herein and that are capable of exhibiting a biological activity of the corresponding full length protein are also encompassed by the present invention.

The protein coding sequence is identified in the sequence listing by translation of the disclosed nucleotide sequences. The mature form of such protein may be obtained by expression of a full-length polynucleotide in a suitable mammalian cell or other host cell. The sequence of the mature form of the protein is also determinable from the amino acid sequence of the full-length form. Where proteins of the present invention are membrane bound, soluble forms of the proteins are also provided. In such forms, part or all of the regions causing the proteins to be membrane bound are deleted so that the proteins are capable of being fully secreted from the cell in which it is expressed.

A variety of methodologies known in the art can be utilized to obtain any one of the isolated polypeptides or proteins of the present invention. At the simplest level, the amino acid sequence can be synthesized using commercially available peptide synthesizers. The polypeptides and proteins of the present invention can alternatively be purified from cells which have been altered to express the desired polypeptide or protein. As used herein, a cell is said to be altered to express a desired polypeptide or protein when the cell, through genetic manipulation, is made to produce a polypeptide or protein which it normally does not produce or which the cell normally produces at a lower level. One skilled in the art can readily adapt procedures for introducing and expressing either recombinant or synthetic sequences into eukaryotic or prokaryotic cells in order to generate a cell which produces one of the polypeptides or proteins of the present invention.

A “fragment” of a polypeptide is meant to refer to any portion of the molecule, such as the peptide core, a variant of the peptide core, or an extracellular region of the polypeptide. A “variant” of a polypeptide is meant to refer to a molecule substantially similar in structure and biological activity to either the entire molecule, or to a fragment thereof Thus, provided that two molecules possess a similar activity, they are considered variants as that term is used herein even if the composition or secondary, tertiary, or quaternary structure of one of the molecules is not identical to that found in the other, or if the sequence of amino acid residues is not identical. An “analogue” of a polypeptide or genetic sequence is meant to refer to a protein or genetic sequence substantially similar in function and structure to the isolated polypeptide or genetic sequence.

It is understood herein that conservative amino acid substitutions can be performed to a purified and isolated polypeptide comprising any one of the sequences of SEQ ID NOS.: 31-44, 46, 48, 50 and 52 which are likely to result in a polypeptide that retains biological or immunological activity, especially if the number of such substitutions is small. By “conservative amino acid substitution” is meant substitution of an amino acid with an amino acid having a side chain of a similar chemical character. Similar amino acids for making conservative substitutions include those having an acidic side chain (glutamic acid, aspartic acid); a basic side chain (arginine, lysine, histidine); a polar amide side chain (glutamine, asparagine); a hydrophobic, aliphatic side chain (leucine, isoleucine, valine, alanine, glycine); an aromatic side chain (phenylalanine, tryptophan, tyrosine); a small side chain (glycine, alanine, serine, threonine, methionine); or an aliphatic hydroxyl side chain (serine, threonine).

Microarrays

Another aspect of the invention is a composition comprising a plurality of polynucleotide probes for use in detecting gene expression pattern(s) characteristic of particular cell type(s) and for detecting changes in the expression pattern of a particular cell type, e.g., lymphatic endothelial cells. For example, the invention comprehends an array, such as a microarray, comprising polynucleotides having at least 10 contiguous nucleotides selected from the polynucleotide sequences presented in the sequence listing.

Also contemplated are microarrays comprising polynucleotides having at least 10 contiguous nucleotides selected from the group of SEQ ID NOS: 1-30, 45, 47, 49 and 51. Microarrays of the invention comprise at least 3 polynucleotides, wherein each enumerated polynucleotide has a distinct sequence selected from the group consisting of SEQ ID NOS:1-30, 45, 47, 49 and 51. Such microarrays may also have duplicate polynucleotides and additional polynucleotides, e.g., control polynucleotides for use in hybridization-based assays using the microarray. Arrays, including microarrays, having more than three distinct polynucleotides according to the invention, such as at least five, seven, nine, 20, 50 or more such polynucleotides, will be recognized as arrays according to the invention having the capability of yielding subtle distinctions between biological samples such as various endothelial cell types, or of providing a different, and typically greater, level of confidence in the various uses of such arrays, e.g., in screening for particular endothelial cells, in screening for abnormal or diseases cells and tissues, and the like.

The term “microarray” refers to an ordered arrangement of hybridizable array elements. The array elements are arranged so that there are preferably at least three or more different array elements, more preferably at least 100 array elements, and most preferably at least 1,000 array elements, on a solid support. Preferably, the solid support is a 1 cm² substrate surface, bead, paper, nylon or other type of membrane, filter, chip, glass slide, or any other suitable solid support. The hybridization signal from each of the array elements is individually distinguishable. In a preferred embodiment, the array elements comprise polynucleotide probes.

Hybridization means contacting two or more nucleic acids under conditions suitable for base pairing. Hybridization includes interaction between partially or perfectly complementary nucleic acids. Suitable hybridization conditions are well known to those of skill in the art. In certain applications, it is appreciated that lower stringency conditions may be required. Under these conditions, hybridization may occur even though the sequences of the interacting strands are not perfectly complementary, being mismatched at one or more positions. Conditions may be rendered less stringent by adjusting conditions in accordance with the knowledge in the art, e.g., increasing salt concentration and/or decreasing temperature. Suitable hybridization conditions are those conditions that allow the detection of gene expression from identifiable expression units such as genes. Preferred hybridization conditions are stringent hybridization conditions, such as hybridization at 42° C. in a solution (ie., a hybridization solution) comprising 50% formamide, 1% SDS, 1 M NaCl, 10% dextran sulfate, and washing for 30 minutes at 65° C. in a wash solution comprising 1×SSC and 0.1% SDS. It is understood in the art that conditions of equivalent stringency can be achieved through variation of temperature and buffer, or salt concentration, as described in Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (2d. Ed.; 1989), pp. 9.47 to 9.51.

One method of using probes and primers of the invention is in the detection of gene expression in human cells. Normally, the target will be expressed RNAs, although genomic DNA or a cDNA library may be screened. By varying the stringency of hybridization and the target binding site (i.e., the sequence of the probe, corresponding to a subset of one of the sequences set forth at SEQ ID NOS: 1-30, 45, 47, 49 and 51), different degrees of homology are expected to result in hybridization.

The microarray can be used for large-scale genetic or gene expression analysis of a large number of target polynucleotides. The microarray can also be used in the diagnosis of diseases and in the monitoring of treatments. Further, the microarray can be employed to investigate an individual's predisposition to a disease. Furthermore, the microarray can be employed to investigate cellular responses to infection, drug treatment, and the like.

The nucleic acid probes can be genomic DNA or cDNA or mRNA polynucleotides or oligonucleotides, or any RNA-like or DNA-like material, such as peptide nucleic acids, branched DNAs, and the like. The probes can be sense or antisense nucleotide probes. Where target polynucleotides are double-stranded, the probes may be either sense or antisense strands. Where the target polynucleotides are single-stranded, the probes are complementary single strands. In one embodiment, the probes are cDNAs. The size of the DNA sequence of interest may vary and is preferably from 100 to 10,000 nucleotides, more preferably from 150 to 3,500 nucleotides.

The probes can be prepared using a variety of synthetic or enzymatic techniques, which are well known in the art. The probes can be synthesized, in whole or in part, using chemical methods well known in the art (Caruthers et al., Nucleic Acids Res., Symp. Ser., 215-233, 1980).

Pharmaceutical Formulations and Routes of Administration

A protein of the present invention (from whatever source derived, such as from recombinant and non-recombinant sources) may be administered to a patient in need, by itself, or in pharmaceutical compositions where it is mixed with suitable carriers, diluents, adjuvants or excipients at doses to treat or ameliorate a variety of disorders. Such a composition may also contain (in addition to protein and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. The pharmaceutical composition of the invention may also contain cytokines, chemokines, lymphokines, growth factors, or other hematopoietic factors such as a PDGF, a VEGF (particularly a VEGF-C or a VEGF-D), VEGFR-3 (including soluble VEGFR-3 peptides comprising an extracellular domain), M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IFN, TNF0, TNF1, TNF2, G-CSF, Meg-CSF, thrombopoietin, stem cell factor, and erythropoietin. Various forms of these polypeptides are contemplated as well, such as isolated holoproteins, subunits, fragments (e.g., soluble fragments), and peptide fusions. The pharmaceutical composition may further contain other agents which either enhance the activity of the protein or complement its activity or use in treatment. Such additional factors and/or agents may be included in the pharmaceutical composition to produce a synergistic effect with a protein of the invention, or to minimize side effects. Conversely, a protein of the present invention may be included in formulations of the particular cytokine, lymphokine, other hematopoietic factor, thrombolytic or anti-thrombotic factor, or anti-inflammatory agent to minimize side effects of the cytokine, lymphokine, other hematopoietic factor, thrombolytic or anti-thrombotic factor, or anti-inflammatory agent. A protein of the present invention may be active in multimers (e.g., heterodimers or homodimers) or complexes with itself or other proteins. As a result, pharmaceutical compositions of the invention may comprise a protein of the invention in such muiltimeric or complexed form.

Techniques for formulation and administration of the compounds of the instant application may be found in “Remington's Pharmaceutical Sciences,” Mack Publishing Co., Easton, Pa., latest edition. A therapeutically effective dose further refers to that amount of the compound sufficient to result in amelioration of symptoms, e.g., treatment, healing, prevention or amelioration of the relevant medical condition, or an increase in rate of beneficial change, healing, prevention or amelioration of such conditions. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

In practicing methods of treatment or use of the present invention, a therapeutically effective amount of protein of the present invention is administered to a mammal having a condition or disorder to be treated. Protein of the present invention may be administered in accordance with the method of the invention either alone or in combination with other therapies such as treatments employing cytokines, lymphokines or other hematopoietic factors. When co-administered with one or more cytokines, lymphokines or other hematopoietic factors, a protein of the invention may be administered either simultaneously with the cytokine(s), lymphokine(s), other hematopoietic factor(s), thrombolytic or anti-thrombotic factors, or sequentially. If administered sequentially, the attending physician will decide on the appropriate sequence of administering a protein of the invention in combination with a cytokine(s), lymphokine(s), other hematopoietic factor(s), thrombolytic or anti-thrombotic factors.

Routes of Administration

Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Administration of protein of the present invention used in the pharmaceutical composition or to practice the method of the present invention is carried out in a variety of conventional ways, such as oral ingestion, inhalation, topical application or cutaneous, subcutaneous, intraperitoneal, parenteral or intravenous injection. Intravenous administration to a mammal, such as a human patient, is preferred. Alternately, one may administer the compound in a local rather than systemic manner, for example, via injection of the compound at the site of intended action.

Compositions/Formulations

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. These pharmaceutical compositions may be manufactured in a manner that is itself known, e.g., by means of conventional mixing, dissolving, granulating, dragee-making, levigating, emulsifying, encapsulating, entrapping or lyophilizing processes. Proper formulation is dependent upon the route of administration chosen. When a therapeutically effective amount of protein of the present invention is administered orally, protein of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% protein of the present invention, and preferably firom about 25 to 90% protein of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of protein of the present invention, and preferably from about 1 to 50% protein of the present invention.

When a therapeutically effective amount of protein of the present invention is administered by intravenous, cutaneous or subcutaneous injection, protein of the present invention will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to protein of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art. For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combination with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory-agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose-in-water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g., polyvinyl pyrrolidone; and other sugars or polysaccharides may substitute for dextrose. Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compound over a time period of a few weeks up to over 100 days. Depending on the chemical nature and the biological stability of the therapeutic reagent, additional strategies for protein stabilization may be employed.

The pharmaceutical compositions also may comprise suitable solid or gel-phase carriers or excipients. Examples of such carriers or excipients include but are not limited to calcium carbonate, calcium phosphate, various sugars, starches, cellulose derivatives, gelatin, and polymers such as polyethylene glycols. Many of the proteinase-inhibiting compounds of the invention may be provided as salts with pharmaceutically compatible counterions. Such pharmaceutically acceptable base addition salts are those salts which retain the biological effectiveness and properties of the free acids and which are obtained by reaction with inorganic or organic bases such as sodium hydroxide, magnesium hydroxide, ammonia, trialkylamine, dialkylamine, monoalkylamine, dibasic amino acids, sodium acetate, potassium benzoate, triethanol amine, and the like.

The pharmaceutical compositions of the invention may be in the form of a complex of a protein(s) of the present invention along with protein or peptide antigens. The pharmaceutical compositions of the invention may be in the form of a liposome in which protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, each of which is incorporated herein by reference.

The amount of protein of the invention in the pharmaceutical composition will depend upon the nature and severity of the condition being treated, and on the nature of prior treatments which the patient has undergone. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further. It is contemplated that the various pharmaceutical compositions used to practice the methods of the invention should contain about 0.01 μg to about 100 mg (preferably about 0.1 μg to about 10 mg, more preferably about 0.1 μg to about 1 mg) of protein of the invention per kg body weight. When administered, the therapeutic composition for use in this invention is in a pyrogen-free, physiologically acceptable form. Therapeutically useful agents other than a protein of the invention which may also optionally be included in the composition as described above, may alternatively or additionally, be administered simultaneously or sequentially with the composition in the methods of the invention.

Polynucleotides of the present invention can also be used for gene therapy. Such polynucleotides can be introduced either in vivo or ex vivo into cells for expression in a mammalian subject. Polynucleotides of the invention may also be administered by other known methods for introduction of nucleic acid into a cell or organism (including, without limitation, in the form of viral vectors or naked DNA). Cells may also be cultured ex vivo in the presence of proteins of the present invention in order to proliferate or to produce a desired effect on or activity in such cells. Treated cells can then be introduced in vivo for therapeutic purposes.

Effective Dosage

Pharmaceutical compositions suitable for use in the present invention include compositions wherein the active ingredients are contained in an effective amount to achieve an intended purpose. More specifically, a therapeutically effective amount means an amount effective to prevent development of, or to alleviate the existing symptoms of, the subject being treated. Suitable properties that may be used in determining effective dosages include measurements of LEC and/or BEC growth stimulation or inhibition, rates or extent of cell differentiation into LECs and/or BECs, tendencies of cell expression patterns to shift towards or away from LEC- or BEC-specific expression patterns, and the like. Determination of the effective amounts is well within the capability of those skilled in the art, especially in light of the detailed disclosure provided herein. For any compound used in a method of the invention, the therapeutically effective dose can be estimated initially from cell culture assays. For example, for inhibitory methods, a dose can be formulated in animal models to achieve a circulating concentration range that includes the IC₅₀ as determined in cell culture (i.e., the concentration of the test compound which achieves a half-maximal inhibitory concentration). Such information can be used to more accurately determine useful doses in humans.

A therapeutically effective dose refers to that amount of the compound that results in amelioration of symptoms or, in the case of life-threatening conditions, a prolongation of survival in a patient. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. See, e.g., Fingl et al., 1975, in “The Pharmacological Basis of Therapeutics”, Ch. 1 p. 1.

The amount of composition administered will, of course, be dependent on the subject being treated, on the subject's weight, the severity of the affliction, the manner of administration and the judgment of the prescribing physician.

Packaging

The compositions may, if desired, be presented in a pack or dispenser device which may contain one or more unit dosage forms containing the active ingredient. The pack may, for example, comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labelled for treatment of an indicated condition.

In addition, the invention comprehends a use of such a composition to manufacture a medicament for the treatment of a cell or an organism, such as a human patient, having a hyperproliferative or hypoproliferative disorder of a LEC and/or BEC, such as lymphedema, lymphangioma, lymphangiomyeloma, lymphangiomatosis, lymphangiectasis, Iymphosarcoma, or lymphangiosclerosis, comprising administering an effective amount, or dose, of a composition according to the invention to the cell or organism. Suitable compositions include, but are not limited to, any polynucleotide according to the invention (e.g., an antisense polynucleotide), any polypeptide according to the invention, an antibody specifically recognizing a polynucleotide or polypeptide according to the invention, a small molecule compound effective in modulating the expression of a polynucleotide according to the invention, and the like. Also contemplated are uses of compositions according to the invention for the manufacture of a medicament to ameliorate a symptom associated with a LEC- or BEC-associated disease or disorder.

Antibodies

Antibodies are useful for modulating the polypeptides of the invention due to the ability to easily generate antibodies with relative specificity, and due to the continued improvements in technologies for adopting antibodies to human therapy. Thus, the invention contemplates use of antibodies (e.g., monoclonal and polyclonal antibodies, single chain antibodies, chimeric antibodies, bifunctional/bispecific antibodies, humanized antibodies, human antibodies, and complementary determining region (CDR)-grafted antibodies, including compounds which include CDR sequences which specifically recognize a polypeptide of the invention), specific for polypeptides of interest to the invention. Preferred antibodies are human antibodies, such as those produced in transgenic animals, which are produced and identified according to methods described in WO93/11236, published Jun. 20, 1993, which is incorporated herein by reference in its entirety. Antibody fragments, including Fab, Fab′, F(ab′)2, and Fv, are also provided by the invention. The term “specific for,” when used to describe antibodies of the invention, indicates that the variable regions of the antibodies of the invention recognize and bind the polypeptide of interest at a detectably different, and greater, level that bind to other substances (i.e., able to distinguish the polypeptides of interest from other known polypeptides of the same family, by virtue of measurable differences in binding affinity, despite the possible existence of localized sequence identity, homology, or similarity between family members). It will be understood that specific antibodies may also interact with other proteins (for example, S. aureus protein A or other antibodies in ELISA techniques) through interactions with sequences outside the variable region of the antibodies, and in particular, in the constant region of the molecule. Screening assays to determine binding specificity of an antibody of the invention are well known and routinely practiced in the art. For a comprehensive discussion of such assays, see Harlow et al. (Eds.), Antibodies A Laboratory Manual; Cold Spring Harbor Laboratory; Cold Spring Harbor, N.Y. (1988), Chapter 6.

Non-human antibodies may be humanized by any method known in the art. A preferred “humanized antibody” has a human constant region, while the variable region, or at least a complementarity-determining region (CDR), of the antibody is derived from a non-human species. Methods for humanizing non-human antibodies are well known in the art. (see U.S. Pat. Nos. 5,585,089, and 5,693,762). Generally, a humanized antibody has one or more amino acid residues introduced into its framework region from a source which is non-human. Humanization can be performed, for example, using methods described in Jones et al. [Nature 321: 522-525, (1986)], Riechmann et al., [Nature, 332: 323-327, (1988)] and Verhoeyen et al. [Science 239:1534-1536, (1988)], by substituting at least a portion of a rodent CDR for the corresponding regions of a human antibody. Numerous techniques for preparing engineered antibodies are described, e.g., in Owens and Young, J. Immunol. Meth., 168:149-165 (1994). Further changes can then be introduced into the antibody framework to modulate affinity or immunogenicity.

The invention further provides a hybridoma that produces an antibody according to the invention. Antibodies of the invention are useful for detection and/or purification of the polypeptides of the invention.

Polypeptides and/or polynucleotides of the invention may also be used to immunize animals to obtain polyclonal and monoclonal antibodies which specifically react with the polypeptide. Such antibodies may be obtained using either the entire polypeptide or fragments thereof as an immunogen. The peptide immunogens additionally may contain a cysteine residue at the carboxyl terminus, and may be conjugated to a hapten such as keyhole limpet hemocyanin (KLH). Methods for synthesizing such peptides are known in the art, for example, as in R. P. Merrifield, J. Amer. Chem. Soc. 85:2149-2154 (1963); J. L. Krstenansky, et al., FEBS Lett. 211: 10 (1987). Monoclonal antibodies binding to the protein of the invention may be useful diagnostic agents for the immunodetection of the polypeptide. Neutralizing monoclonal antibodies binding to the polypeptide may also be useful therapeutics for both conditions associated with the polypeptide and also in the treatment of some forms of cancer where abnormal expression of the polypeptide is involved. In the case of cancerous cells or leukemic cells, neutralizing monoclonal antibodies against the polypeptide are useful in detecting and preventing the metastatic spread of the cancerous cells mediated by the polypeptide. In general, techniques for preparing polyclonal and monoclonal antibodies as well as hybridomas capable of producing the desired antibody are well known in the art (Campbell, A. M., Monoclonal Antibodies Technology: Laboratory Techniques in Biochemistry and Molecular Biology, Elsevier Science Publishers, Amsterdam, The Netherlands (1984); St. Groth et al., J. Immunol. 35:1-21 (1990); Kohler and Milstein, Nature 256:495-497 (1975)), the trioma technique, the human B-cell hybridoma technique (Kozbor et al., Immunology Today 4:72 (1983); Cole et al., in Monoclonal Antibodies and Cancer Therapy, Alan R. Liss, Inc. (1985), pp. 77-96).

Any animal (mouse, rabbit, and the like) which is known to produce antibodies can be immunized with a peptide or polypeptide of the invention. Methods for immunization are well known in the art. Such methods include subcutaneous or intraperitoneal injection of the polypeptide. One skilled in the art will recognize that the amount of the polypeptide encoded by an ORF of the invention used for immunization will vary based on the animal which is immunized, the antigenicity of the peptide and the site of injection. The protein that is used as an immunogen may be modified or administered in an adjuvant in order to increase the protein's antigenicity. Methods of increasing the antigenicity of a protein are well known in the art and include, but are not limited to, coupling the antigen with a heterologous protein (such as a globulin or β-galactosidase) or through the inclusion of an adjuvant during immunization.

For monoclonal antibodies, spleen cells from the immunized animals are removed, fused with myeloma cells, such as SP2/0-Ag14 myeloma cells, and allowed to become monoclonal-antibody-producing hybridoma cells. Any one of a number of methods well known in the art can be used to identify the hybridoma cell which produces an antibody with the desired characteristics. These include screening the hybridomas with an ELISA assay, Western blot analysis, or radioimmunoassay (Lutz et al., Exp. Cell Research. 175:109-124. 1988). Hybridomas secreting the desired antibodies are cloned and the class and subclass is determined using procedures known in the art (Campbell, A. M., Monoclonal Antibody Technology: Laboratory Techniques in Biochemistry and Molecular Biology, Elsevier Science Publishers, Amsterdam, The Netherlands (1984)). Techniques described for the production of single-chain antibodies (U.S. Pat. No. 4,946,778) can be adapted to produce single-chain antibodies to polypeptide of the invention.

For polyclonal antibodies, antibody-containing antiserum is isolated from the immunized animal and is screened for the presence of antibodies with the desired specificity using one of the above-described procedures. The invention further provides the above-described antibodies in detectably labeled form. Antibodies can be detectably labeled through the use of radioisotopes, affinity labels (such as biotin, avidin, and the like), enzymatic labels (such as horseradish peroxidase, alkaline phosphatase, and the like) fluorescent labels (such as FITC or rhodamine, and the like), paramagnetic atoms, and the like. Procedures for accomplishing such labeling are well-known in the art; for example, see Stemberger, L. A. et al., J. Histochem. Cytochem. 18:315. 1970; Bayer, E. A. et al., Meth. Enzym. 62:308 (1979); Engval, E. et al., Immunol. 109:129. 1972; and Goding, J. W. J. Immunol. Meth. 13:215. (1976).

The labeled antibodies of the present invention can be used for in vitro, in vivo, and in situ assays to identify cells or tissues in which a fragment of the polypeptide of interest is expressed. The antibodies may also be used directly in therapies or other diagnostics. The present invention further provides the above-described antibodies immobilized on a solid support. Examples of such solid supports include plastics such as polycarbonate, complex carbohydrates such as agarose and sepharose, and acrylic resins such as polyacrylamide and latex beads. Techniques for coupling antibodies to such solid supports are well known in the art (Weir, D. M. et al., “Handbook of Experimental Immunology” 4th Ed., Blackwell Scientific Publications, Oxford, England, Chapter 10 (1986); Jacoby, W. D. et al., Meth. Enzym. 34 Academic Press, N.Y. (1974)). The immobilized antibodies of the present invention can be used for in vitro, in vivo, and in situ assays as well as for immuno-affinity purification of the proteins of the present invention.

Computer-Readable Sequences

In one application of this embodiment, a nucleotide sequence of the present invention can be recorded on computer-readable media. As used herein, “computer-readable media” refers to any medium which can be read and accessed directly by a computer. Such media include, but are not limited to, magnetic storage media, such as floppy discs, hard disc storage medium, and magnetic tape; optical storage media such as CD-ROM; electrical storage media such as RAM and ROM; and hybrids of these categories such as magnetic/optical storage media. A skilled artisan can readily appreciate how any of the presently known computer-readable media can be used to create a manufacture comprising computer-readable medium having recorded thereon a nucleotide sequence of the present invention. As used herein, “recorded” refers to a process for storing information on computer-readable medium. A skilled artisan can readily adopt any of the presently known methods for recording information on a computer-readable medium to generate manufactures comprising the nucleotide sequence information of the present invention.

A variety of data storage structures are available to a skilled artisan for creating a computer-readable medium having recorded thereon a nucleotide sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. A skilled artisan can readily adapt any number of data processor structuring formats (e.g., text file or database) in order to obtain computer readable medium having recorded thereon the nucleotide sequence information of the present invention. By providing the nucleotide sequence of SEQ ID NO: 1-30, 45, 47, 49 and 51 or a representative fragment thereof, or a nucleotide sequence at least 99.9% identical to SEQ ID NO: 1-30, 45, 47, 49 and 51 in computer-readable form, a skilled artisan can routinely access the sequence information for a variety of purposes. Computer software is publicly available which allows a skilled artisan to access sequence information provided in a computer-readable medium. The examples which follow demonstrate how software which implements the BLAST (Altschul et al., J. Mol. Biol. 215:403-410. 1990) and BLAZE (Brutlag et al., Comp. Chem. 17:203-207 (1993)) search algorithms on a Sybase system is used to identify open reading frames (ORFs) within a nucleic acid sequence. Such ORFs may be protein-encoding fragments and may be useful in producing commercially important proteins such as enzymes used in fermentation reactions and in the production of commercially useful metabolites.

As used herein, “a computer-based system” refers to the hardware means, software means, and data storage means used to analyze the nucleotide sequence information of the present invention. The minimum hardware means of the computer-based systems of the present invention comprises a central processing unit (CPU), input means, output means, and data storage means. A skilled artisan can readily appreciate that any one of the currently available computer-based systems are suitable for use in the invention. As stated above, the computer-based systems of the present invention comprise a data storage means having stored therein a nucleotide sequence of the present invention and the necessary hardware means and software means for supporting and implementing a search means. As used herein, “data storage means” refers to memory which can store nucleotide sequence information of the present invention, or a memory access means which can access manufactures having recorded thereon the nucleotide sequence information of the present invention.

As used herein, “search means” refers to one or more programs which are implemented on the computer-based system to compare a target sequence or target structural motif with the sequence information stored within the data storage means. Search means are used to identify fragments or regions of a known sequence which match a particular target sequence or target motif. A variety of known algorithms are disclosed publicly and a variety of commercially available software for conducting search means are and can be used in the computer-based systems of the present invention. Examples of such software includes, but is not limited to, MacPattern (EMBL), BLASTN and BLASTA (NPOLYPEPTIDEIA). A skilled artisan can readily recognize that any one of the available algorithms or implementing software packages for conducting homology searches can be adapted for use in the present computer-based systems. As used herein, a “target sequence” can be any nucleic acid or amino acid sequence of six or more nucleotides or two or more amino acids. A skilled artisan can readily recognize that the longer a target sequence is, the less likely a target sequence will be present as a random occurrence in the database. The most preferred sequence length of a target sequence is from about 10 to 100 amino acids or from about 30 to 300 nucleotide residues. However, it is well recognized that searches for commercially important fragments, such as sequence fragments involved in gene expression and protein processing, may be of shorter length.

As used herein, “a target structural motif,” or “target motif,” refers to any rationally selected sequence or combination of sequences in which the sequence(s) are chosen based on a three-dimensional configuration which is formed upon the folding of the target motif. There are a variety of target motifs known in the art. Protein target motifs include, but are not limited to, enzyme active sites and signal sequences. Nucleic acid target motifs include, but are not limited to, promoter sequences, hairpin structures and inducible expression elements (protein binding sequences).

Diagnostic Assays and Kits

The present invention further provides diagnostic assays, and related kits, for hyper- and/or hypo-proliferative disorders or diseases of endothelial cells such as LECs or BECs. These assays comprise methods to identify the presence or expression of one of the ORFs of the present invention, or homolog thereof, in a test sample, using a nucleic acid probe or an antibody according to the invention.

In general, methods for detecting a polynucleotide of the invention can comprise contacting a sample with a compound that binds to and forms a complex with, the polynucleotide for a period sufficient to form the complex, and detecting the complex, so that if a complex is detected, a polynucleotide of the invention is detected in the sample.

Such methods can also comprise contacting a sample under stringent hybridization conditions with nucleic acid primers that anneal to a polynucleotide of the invention under such conditions, and amplifying annealed polynucleotides, so that if a polynucleotide is amplified, a polynucleotide of the invention is detected in the sample.

In general, methods for detecting a polypeptide of the invention can comprise contacting a sample with a compound that binds to and forms a complex with the polypeptide for a period sufficient to form the complex, and detecting the complex, so that if a complex is detected, a polypeptide of the invention is detected in the sample. In detail, such methods comprise incubating a test sample with one or more of the antibodies or one or more of the nucleic acid probes of the invention and assaying for binding of the nucleic acid probes or antibodies to components within the test sample.

Conditions for incubating a nucleic acid probe or antibody with a test sample vary. Incubation conditions depend on the format employed in the assay, the detection methods employed, and the type and nature of the nucleic acid probe or antibody used in the assay. One skilled in the art will recognize that any one of the commonly available hybridization, amplification or immunological assay formats can readily be adapted to employ the nucleic acid probes or antibodies of the present invention. Examples of such assays can be found in Chard, T., An Introduction to Radioimmunoassay and Related Techniques, Elsevier Science Publishers, Amsterdam, The Netherlands (1986); Bullock, G. R. et al., Techniques in Inmunocytochemistry, Academic Press, Orlando, Fla. Vol. 1 (1982), Vol. 2 (1983), Vol. 3 (1985); Tijssen, P., Practice and Theory of immunoassays: Laboratory Techniques in Biochemistry and Molecular Biology, Elsevier Science Publishers, Amsterdam, The Netherlands (1985). The test samples of the present invention include cells, protein or membrane extracts of cells, or biological fluids such as sputum, blood, serum, plasma, or urine. The test sample used in the above-described method will vary based on the assay format, nature of the detection method and the tissues, and cells or extracts used as the sample to be assayed. Methods for preparing protein extracts or membrane extracts of cells are well known in the art and can be readily be adapted in order to obtain a sample which is compatible with the system utilized.

In another embodiment of the present invention, kits are provided which contain the necessary reagents to carry out the assays of the present invention. In one embodiment, the invention provides a compartment kit to receive, in close confinement, one or more containers which comprises: (a) a first container comprising one of the probes or antibodies of the present invention; and (b) one or more other containers comprising one or more of the following: wash reagents, reagents capable of detecting presence of a bound probe or antibody.

In detail, a compartment kit includes any kit in which reagents are contained in separate containers. Such containers include small glass containers, plastic containers or strips of plastic or paper. Such containers allows one to efficiently transfer reagents from one compartment to another compartment such that the samples and reagents are not cross-contaminated, and the agents or solutions of each container can be added in a quantitative fashion from one compartment to another. Such containers will include a container which will accept the test sample, a container which contains the antibody or antibodies used in the assay, containers which contain wash reagents (such as phosphate-buffered saline, Tris buffers, and the like), and containers which contain the reagents used to detect the bound antibody or probe. Types of detection reagents include labeled nucleic acid probes, labeled secondary antibodies, or in the alternative, if the primary antibody is labeled, the enzymatic, or antibody binding reagents which are capable of reacting with the labeled antibody. One skilled in the art will readily recognize that the disclosed probes and antibodies of the present invention can be readily incorporated into one of the established kit formats which are well known in the art.

### EXAMPLES

Methods used in the examples are as follows:

Antibodies

Monoclonal antibodies against human VEGFR-3 (clone 2E11D11; see International Patent Application No. PCT/US02/22164, published as WO 03/006104), PAL-E (Monosan), CD31 (Dako), N-cadherin, VE-cadherin, β-catenin and plakoglobin and polyclonal rabbit anti-human podoplanin were used (Breiteneder-Geleff, S., et al., Am. J. Pathol. 154:385-394 (1999)); Mouse anti-human integrin α9 was provided by Dr. Dean Sheppard (University of California at San Francisco, San Francisco) and Dr. Curzio Rüegg (University of Lausanne Medical School, Lausanne, Switzerland). The fluorochrome-conjugated secondary antibodies were obtained from Jackson Immunoresearch.

Cell Culture and Transfection

Human amniotic epithelial cells were cultured in Med199 medium in the presence of 5% fetal calf serun. Human dermal microvascular endothelial cells were obtained from PromoCell (Heidelberg, Germany). Anti-Podoplanin antibodies, MACS colloidal super-paramagnetic MicroBeads conjugated to goat anti-rabbit IgG antibodies (Miltenyi Biotech, Bergisch Gladbach, Germany), LD and MS separation columns and Midi/MiniMACS separators (Miltenyi Biotech) were used for cell sorting according to the instructions of the manufacturer. The isolated cells were cultured on fibronectin-coated (10 μg/ml, Sigma, St. Louis, Mo.) plates as described (Mäkinen, T., et al., EMBO J. 20:4762-4773. 2001).

RNA Isolation, Northern Blotting and Microarray Analyses

Total RNA was isolated and DNAseI treated in RNeasy columns (Qiagen, Valencia, Calif.). ³²P-labeled probes for hybridization with the Atlas filters (Clontech) were prepared using 2-5 μg of total RNA according to the manufacturer's instructions with the exception that the probe was purified using Nick-25 columns (Pharmacia Biotech, Uppsala, Sweden). Following hybridizations and washes, the membranes were analyzed using a Fuji BAS 100 phosphoimager. For the Affymetrix® analysis, four independent BEC and LEC sample preparations and hybridizations were carried out using RNA extracted from four lots of cells isolated from different individuals. For the Affymetrix® expression analysis, 5 μg of total RNA was used for the synthesis of double-stranded cDNA using Custom SuperScript ds-cDNA Synthesis Kit (Invitrogen, Carlsbad, Calif.). Biotin-labeled cRNA was then prepared using the Enzo BioArrayTMHighYieldTMRNA Transcript Labelling Kit (Affymetrix, Santa Clara, Calif.), and the unincorporated nucleotides were removed using RNeasy columns (Qiagen, Valencia, Calif.). The hybridization, washing and staining of Human Genome 95Av2 microarrays (for Prox-1 experiments) and 9513-E microarrays, which mainly contain uncharacterized EST sequences, were done according to the instructions of the manufacturer (Affymetrix, GeneChip Expression Analysis Technical Manual). The probe arrays were scanned at 570 nm using an Agilent GeneArray® Scanner and the readings from the quantitative scanning were analyzed by the Affymetrix® Microarray Suite version 5.0 and Data Mining Tool version 3.0. For the comparison analyses, the hybridization intensities were calculated using a global scaling intensity of 100.

The differentially expressed sequences were used for searching EST contigs in the GenBank database of the National Center for Biotechnology Information and the National Library of Medicine. (NCBI/NLM), and open reading frames were predicted using the orf finder software available at NCBI/NLM. The SOSUI system was used for prediction of transmembrane helices and signal sequences from the protein sequences, and other protein domain architectures were analysed using Pfam (Protein families database of alignments and HMMs).

Immunofluorescence and Immunohistochemistry

The cells were cultured on coverslips, fixed with 4% paraformaldehyde, permeabilized with 0.1% Triton-X100 in phosphate-buffered saline (PBS) and stained with the primary antibodies. For integrin α9, staining live cells were incubated with the antibody for 15 minutes on ice before fixation. The cells were further stained with FITC- or TRITC-conjugated secondary antibodies. F-actin was stained using TexasRed-conjugated phalloidin (Molecular Probes, Eugene, Oreg.). Cells were counterstained with Hoechst 33258 fluorochrome (Sigma) and viewed using a Zeiss Axioplan 2 fluorescent microscope.

Normal human skin obtained after surgical removal was embedded in Tissue-Tek® (Sakura, The Netherlands), frozen and sectioned. The sections (6 μm) were fixed in cold acetone for 10 minutes and stained with the primary antibodies followed by peroxidase staining using Vectastain Elite ABC kit (Vector Laboratories, Burlingame, Calif.) and 3-amino-9-ethyl carbazole (Sigma, St. Louis, Mo.).

### Example 1

### Identification of Differentially Expressed Genes

Blood vascular and lymphatic endothelial cells (BEC and LEC, respectively) were isolated from cultures of human dermal microvascular endothelial cells using magnetic microbeads and antibodies against the lymphatic endothelial cell surface marker podoplanin (Breiteneder-Geleff, S., et al., Am. J. Pathol. 154:385-394 (1999); Makinen, T., et al., EMBO J. 20:4762-4773 (2001)). The purities of the isolated BEC and LEC populations were confirmed to be over 99% as assessed by immunofluorescence using antibodies against VEGFR-3 or podoplanin. The isolated cells were cultured for a couple of passages, and RNA was extracted from the cultures and used for hybridization with oligonucleotide microarrays containing sequences from about 12,000 known genes, ie., approximately ⅓ of the total number of all predicted human transcripts.

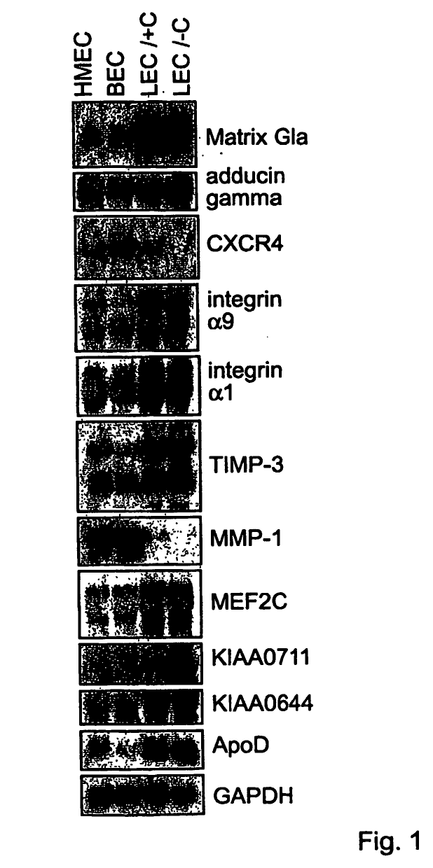

As expected, podoplanin, desmoplakin I/II and the macrophage mannose receptor, which are known lymphatic endothelial cell markers, were found specifically in the LECs. See, Breiteneder-Geleff, S., et al., Am. J. Pathol. 154:385-394 (1999); Ebata, N., et al., Microvasc. Res. 61:40-48. (2001); and Irjala, H., et al., J. Exp. Med. 194:1033-1041 (2001). Since these results were consistent with the known gene expression patterns in vivo and in vitro, further characterization of the gene expression profiles was carried out. When a reproducible signal log₂ ratio of 1.0 (twofold difference) was selected in the replicate analyses, over 400 genes were found to be differentially expressed between LECs and BECs. Some examples of the differentially expressed genes have been functionally annotated in Table 1 and a complete list of the differentially expressed genes is provided in Tables 2-4. A complete list of differentially expressed genes containing the GenBank accession numbers and the variation between the expression levels between independently harvested BECs and LECs (signal log₂ ratio ±s.d.) are provided in Tables 3 and 4. The microarray data were validated by Northern blotting or by immunofluorescence for 31 of the selected genes (see FIG. 1).

Each gene listed in Tables 3 and 4 is identified by a gene accession number which correlates to the sequence of the gene as found in a public genome database such as the GenBank database maintained by NCBI. These sequences are incorporated herein by reference.

TABLE 1
Selected classes of genes differentially expressed in BECs and LECs.

| | Blood vascular EC | Lymphatic EC |
|---|---|---|
| Adhesion molecules | integrin alpha5 | integrin alpha9* |
| | integrin β5, β4* | integrin alpha1 |
| | ICAM-1*, ICAM-2 | macrophage mannose receptor I* |
| | N-cadherin* | |
| | selectin P, selectin E* | |
| | protocadherin 42* | |
| | CD44* | |
| | EphrinB1* | |
| Cytoskeletal proteins | vinculin | desmoplakin I and II* |
| | claudin 7* | adducin gamma |
| | actin, alpha 2 | alpha-actinin-2 associated LIM |
| | profilin 2 | protein* |
| ECM proteins | collagens 8A1*, 6A1*, 4A2/13A1*, | Matrix Gla protein* |
| | 1A2* | |
| | laminin* | |
| | versican* | |
| | proteoglycan 1 | |
| ECM modulation | MMP-1, MMP-10, MMP-14* | TIMP-3 |
| | uPA*, tPA* | |
| | cathepsin C | |
| Receptor tyrosine | VEGFR-1 (sVEGFR-1*) | VEGFR-3* |
| kinases | | Lyn |
| and other protein | | Dyrk3 |
| kinases | | |
| Transcription factors | STAT6* | prox-1* |
| | TFEC* | MEF2C* |
| | MAD-3* | c-maf* |
| | HMGI-C* | forkhead box M1 |
| | JUN* | CREM |
| | GATA2 | ear-3 |
| Growth factors | VEGF-C* | Angiopoietin-2 |
| | Placenta growth factor | |
| Cytokines, chemokines | IL-8*, IL-6* | IL-7* |
| and receptors | stem cell factor* | SDF-1b* |
| | Monocyte chemotactic protein 1 | |
| | UFO/axl* | |
| | CXCR4 | |
| | CCRL2/CKRX* | |
| | IL-4 receptor | |
| Cell cycle | p27* | Cdk-inhibitor p57KIP2* |
| | p21 | cyclin-dependent kinase inhibitor 3, |
| | gadd45 | CIP2 |
| | | cyclin E2* |
| | | cyclin B1, B2* |
| Oxidative stress | thioredoxin reductase beta* | selenoprotein P* |
| Other | Neuropilin-1 | podoplanin* |
| | HNMP-1* | MRC OX2 |
| | endothelial cell protein C/APC | Apolipoprotein D |
| | receptor | Semaphorin 3A* |
| | Rnase A, pancreatic* | fatty acid binding protein 4 |
| | TGF-β | LITAF/Pig7* |
| | LTBP-2 | IGFBP-2* |
| | metallothionein I, II, III | piccolo* |
| | Cyclooxygenase 2* | monoamine oxidase A |
| | clusterin/Apolipoprotein J | neuronal pentraxin II* |
| | neuronal pentraxin I* | |
| Total | 222 genes | 187 genes |

Genes shown in bold were confirmed by Northern blotting or immunofluorescence, and those marked with an asterisk (*) were specifically expressed in only one of the two cell lineages.

TABLE 2
Known LEC-specific genes

| | | Accession numbers | |
|---|---|---|---|
| Gene | Detection* | starting EST | possible gene |
| CD36 = | Af (S/4, 3) | R20784 | M98399 |
| COL1/TSP receptor, fatty-acid transport | | H54254 | |
| protein | | | |
| beta1-syntrophin | Af (S/4, 5) | AA447177 | L31529 |
| collectin sub-family member 12 | Af (S/4, 5) | R74387 | NM_030781 |
| a disintegrin and metalloprotease | Af (S/4, 3) | AA147933 | NM_003474 |
| domain 12 | | | |
| cytotoxic T-lymphocyte- | Af (S/4, 0) | AI733018 | NM_005214 |
| associated protein 4 | | | |
| niban protein NM_022083 niban | Af (S/3, 7) | AA554814 | NM_052966 |
| protein | | | |
| multi-PDZ-domain-containing | Af (S/3, 5) | AI738919 | NM_032622 |
| protein, LNX | | | |
| MAGE-E1 protein | Af (S/3, 2) | AI435112 | NM_030801 |
| upstream stimulatory factor 1, | Af (S/2, 6) | AA701033 | AB017568 |
| USF1 (genomic match) | | | |
| hairy/enhancer-of-split related | Af (NS/2, 6) | R61374 | NM_012258 |
| with YRPW motif 1 | | | |
| alpha-2,8-polysialyltransferase | Af (S/2, 5) | AI422986 | L41680 |
| semaphorin 6A1 | Af (S/2, 4) | W21965 | NM_020796 |
| guanine nucleotide binding | Af (S/2, 3) | AA738022 | |
| protein (G prot), gamma 2 | | | |
| integral membrane protein 3 | Af (S/2, 3) | AA128019 | NM_030926 |
| similar to mouse glucocorticoid- | Af (S/2, 0) | AI678080 | XM_070471 |
| induced gene 1 | | | |
| YAP65 (Yes-associated protein of | Af (NS/2, 0) | AL048399 | X80507 |
| 65 kDa MW) | | | |
| 17 kDa fetal brain protein | Af (NS/1, 9) | H92988 | NM_022343 |
| Kruppel-like factor 5 | Af (S/1, 8) | AI815057 | NM_001730 |
| calcitonin receptor-like, CGRP | Af (S/1, 7) | AI741128, | NM_005795, L76380 |
| type 1 receptor | | T94540 | |
| fibroblast growth factor 13, | Af (NS/1, 7) | AW014749 | NM_004114 |
| isoform 1A | | | |
| tetraspan NET-6 protein | Af (NS/1, 6) | W22687 | NM_014399 |
| ring finger protein 11 | Af (S/1, 6) | AL079648 | BC020964 |

*Af = Affymetrix, S = specific for LEC, NS = nonspecific (also expressed in BEC), numbers represent log₂ ratio of the signal intensities between BEC and LEC

### Example 2

### Bec-Specific Expression of Genes Involved in Inflammation

Endothelial cells play an important role in several steps of the inflammatory response. They recruit leukocytes to inflammatory foci and specialized endothelial cells (high endothelial venules) are responsible for the homing of lymphocytes to the secondary lymphoid organs. In addition, endothelial cells modulate leukocyte activation and vice versa, and they can become activated by molecules secreted by the leukocytes. Consistent with their activation in cell culture, the BECs expressed high levels of pro-inflammatory cytokines and chemokines (stem cell factor, interleukin-8, monocyte chemotactic protein 1 (MCP-1)) and receptors (UFO/axl, CXCR4, IL-4R) see Table I. CXCR4 and its ligand, stromal cell-derived factor-1 (SDF-1), play important roles in the trafficking of normal lymphocytes, monocytes, and hematopoietic stem- and progenitor cell, targeted inactivation of either CXCR4 or SDF-1 results in impaired cardiogenesis, hematopoiesis and vascular development (Tachibana, et al., Nature 393:591-594. 1998). SDF-1b was mainly produced by the LECs, suggesting that this chemokine may be involved in LEC-initiated chemotaxis of the CXCR4-expressing cells. Moreover, the reciprocal pattern of expression of CXCR4 and SDF-1 on BECs and LECs suggest that the two cell types use these molecules for paracrine communication.

### Example 3

### Differences in Cell Adhesion, Cell-Cell Interaction and Cytoskeletal Molecules

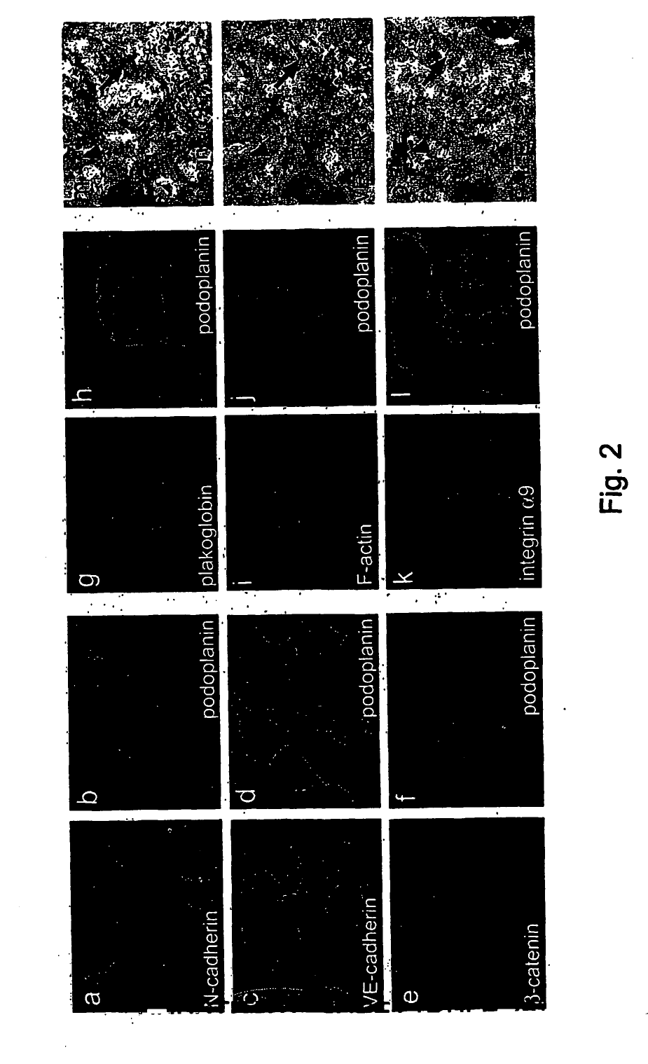

The most striking differences detected between the BECs and LECs was the expression of genes involved in cytoskeletal and cell-cell or cell-matrix interactions (see Tables 3 and 4). For example, N-cadherin, which is involved in the interaction of endothelial cells with SMCs and pericytes (Gerhardt, et al., Dev. Dyn. 218:472-479. 2000), was detected specifically in BECs. This is consistent with the fact that the lymphatic capillaries are not ensheathed by SMCs. In immunostaining, N-cadherin was detected exclusively in the BECs, whereas VE-cadherin was present in both cell types (FIG. 2a-d). The cytoplasmic domains of cadherins interact with β-atenin, plakoglobin (γ-catenin) and p120^{ctn}, which link them to the actin cytoskeleton via α-actinin, vinculin, ZO-1, ZO-2 and spectrin (Provost, E. & Rimm, Curr Op. Cell Biol. 11:567-572. 1999). BECs expressed significantly higher levels of β-catenin (FIG. 2e,f) and vinculin, whereas plakoglobin was mostly present on LECs (FIG. 2g,h). Staining of LECs and BECs also revealed a strikingly different organization of the actin cytoskeleton. BECs displayed numerous stress fibers, which in LECs were almost totally absent, and instead a cortical distribution of actin was observed in LECs(FIG. 2i,j).

Integrins are important mediators of cell adhesion (Giancotti & Ruoslahti, Science 285:1028-1032. 1999). They are transmembrane proteins consisting of two polypeptides, the α and β subunits. Their ectodomains bind extracellular matrix proteins while the cytoplasmic domains interact with the cytoskeleton and with proteins involved in signal transduction. Integrin α5, which acts as a subunit of the fibronectin receptor, mainly was expressed in BECs. By contrast, integrins α1 and α9, which provide subunits for the receptors for laminin and collagen and for osteopontin and tenascin, respectively, were expressed in LECs (FIG. 1a and FIG. 2k,l). In human skin, antibodies against integrin α9 stained lymphatic capillaries specifically, while blood vessel endothelia were negative (FIG. 2m-o). In addition, integrin α9 was detected in arterial smooth muscle cells as previously reported (Palmer, et al., J. Cell Biol. 123:1289-1297. 1993). Interestingly, integrin α9 has been shown to be important for the normal development of the lymphatic system. Mice lacking integrin α9β1 develop respiratory failure due to the accumulation of a milky pleural (presumably lymphatic) effusion and die within 6 to 12 days after birth (Huang, et al., Mol. Cell Biol. 20:5208-5215. 2000).

BECs, but not LECs, produced both laminin and different types of collagens (Table 4). In co-culture these basement membrane components may be necessary for the adhesion and growth of the LECs (Makinen, T., et al., EMBO J. 20:4762-4773. 2001). In addition, many of the proteins involved in matrix degradation and remodeling, including several matrix metalloproteinases, tissue-type and urokinase plasminogen activator, as well as plasminogen activator inhibitor I were detected mainly in BECs, while the tissue inhibitor of matrix metalloproteinases-3 (TIMP-3) was detected mainly in LECs (Table 3 and FIG. 1). Unlike the other TlMPs, which are soluble, TIMP-3 is a component of the extracellular matrix. Recombinant TIMP-3 has been reported to inhibit endothelial cell migration and tube formation in response to angiogenic factors, and when expressed in a tumor model, it inhibited tumor growth most likely by preventing tumor expansion, release of growth factors from the extracellular matrix, or angiogenesis (Anand-Apte, et al., Biochemistry & Cell Biology 74:853-862. 1996).

Additional previously unknown genes were identified in the microarray as LEC-specific transcription factors or transmembrane proteins. See Tables 5 and 6.

TABLE 5
Transcription Factors Identified

| | | Accession numbers | |
|---|---|---|---|
| | | | possible |
| Gene | Detection* | starting EST | gene |
| Homologous to Iroquois related | Af (S/4, 2) | AA936528 | not cloned |
| homeobox 2 | | | from |
| | | | human ( [symbol] |
| similar to mouse odd-skipped | Af (S/3, 3) | AI809953 | (19) |
| related 1 zinc-finger TF | | | |
| PAC clone RP4-751H13 from | Af (S/2, 3) | AC004877 | |
| 7q35-qter | | | |
| similar to mouse glucocorticoid- | Af (NS/2) | AI678080 | XM_070471 |
| induced gene 1 | | | |

*Af = Affymetrix, S = specific for LEC, NS = nonspecific (also expressed in BEC), numbers represent log2 ratio of the signal intensities between BEC and LEC

TABLE 6
Transmembrane Proteins Identified

| | | Accession numbers | |
|---|---|---|---|
| Gene | Detection* | starting EST | possible gene |
| KIAA0626 | Af (S/4, 7) | AB014526 | NM_021647 (14) |
| KIAA0644 | Af (S/3, 9) | AB014544 | NM_014817 (15) |
| unknown protein | Af (S/3, 5) | AI333655 | XM_059074 (16) |
| hypothetical protein | Af (NS/1, 8) | AI733570 | NM_024600 (862) |
| FLJ20898 similar to | Af (NS/1, 7) | AA447940 | AK055654, |
| layilin, unnamed | | | XM_084655 |
| protein product | | | |
| hypothetical protein | Af (NS/3, 2) | AI681538 | NM_022068 (860) |
| FLJ23403 KIAA0062 | | D31887 | XM_046677 (47) |
| mesenchymal stem | Af (S/1, 8) | AW009871 | NM_016647 (17) |
| cell protein DSCD75 | | | |

*Af = Affymetrix, S = specific for LEC, NS = nonspecific (also expressed in BEC), numbers represent log2 ratio of the signal intensities between BEC and LEC

Additionally, Tables 10 and 11 describe the known LEC genes identified and their accession numbers, and the differentially expressed genes and their accession numbers, respectively, while Table 12 describes other unknown proteins identified in the screen.

### Example 4

### Differential Regulation of LEC Genes by Prox-1

The mechanisms responsible for the lymphatic differentiation program were investigated. The Prox-1 homeobox transcription factor was found to be expressed specifically in LECs and targeted disruption of Prox-1 in mice was reported to result in the arrest of lymphatic vessel development (Wigle et al., Cell, 98:769-778. 1999). Despite the fact that the prox-1 gene was discovered nearly ten years ago, Prox-1 target genes have not been identified. To determine whether the homeodomain transcription factor Prox-1 contributes to the differentiated LEC and BEC phenotypes, the genes identified above were analyzed for expression in primary BECs and LECs, in the presence and absence of Prox-1 over-expression.

Adenovirus-mediated gene transfer of prox-1 in primary endothelial cells was used to induce gene expression in the BEC cells. In order to eliminate gene expression changes caused by adenoviral infection, AdLacZ (encoding β-galactosidase) was introduced into BECs as a control.

A prox-1 cDNA was amplified by RT-PCR using total RNA from human endothelial cells and the primers 5′-GCCATCTAGACTACTCATGAAGCAGCT-3′ (SEQ ID NO: 61) and 5′-GCGCAGAATTCGGCCCTGACCATGACAGCACA-3′ (SEQ ID NO: 62). The PCR product was cloned into the pAMC expression vector, producing N-terminally Myc-tagged Prox-1. The construct was then subcloned into pAdCMV to yield AdProx-1 for adenovirus production. AdProx-1 and AdLacZ virus stocks were produced as described (Laitinen et al., Hum. Gene Ther. 9:1481-1486. 1998). Adenovirally produced Prox-1 migrated with a molecular weight of about 85 kDa and it was also recognized by antibodies against a Prox-1 C-terminal peptide. Mutant Prox-1 N625A/R627A, (asparagine to alanine change at codon 625, arginine to alanine change at codon 627) was made using the QuikChange site-directed mutagenesis kit (Stratagene, La Jolla, Calif.) and the following primers:

(SEQ ID NO: 63)

5′-CTCATCAAGTGGTTTAGCGCTTTCCGTAGTTTTACTAC-3′
and

(SEQ ID NO: 64)

5′-GTAGTAAAACTCACGGAAGCGCTAAACCACTTGATGAG-3..

Human dermal microvascular endothelial cells, coronary artery endothelial cells (CAECs), saphenous vein endothelial cells (SAVECs), BECs and LECs were plated 24 hours before adenoviral infection at a density of 8,000 cells/cm² and infected for 1 hour in serum-free medium at 50-100 PFU/cell. At the end of the incubation period the cells were washed and then cultured in complete medium for 20-24 hours. Total RNA isolation and array hybridization were performed as described above.

Titration experiments showed that infection of human microvascular endothelial cells with AdProx-1 or AdLacZ led to nuclear expression of the adenovirus-encoded protein in >90% of the cells at 24 hours post-infection. To investigate the changes in gene expression induced by Prox-1, human cDNA filter arrays were used, which contain about 1,000 genes-known to be important for general cellular metabolism as wells as genes specifically implicated in the regulation of cardiovascular function or hematopoiesis. AdProx-1 up-regulated the expression of 28 LEC genes and down-regulated 63 BEC genes, (see Table 7 below), which was confirmed by Northern blotting for 10 of 11 selected genes. When compared with genes differentially expressed in LECs and BECs, 15 genes (i.e., about 30%) modulated by Prox-1 were found to be differentially expressed between cultured LECs and BECs, suggesting that Prox-1 is a major regulator of lymphatic endothelial cell identity.

TABLE 7
Prox-1 Regulated LEC/BEC Genes

| | | | ¹signal log | |
|---|---|---|---|---|
| Gene | Accession numbers | | ratio | ²s.d. |
| LEC-specific induced by AdProx-1 (28 genes) | | | | |
| cyclin E2 | AF091433 | NM_057735 | 4.95 | 1.17 |
| cysteine and glycine-rich protein 2 | U57646 | NM_001321 | 4.58 | 0.36 |
| Cdk-inhibitor p57KIP2 | U22398 | NM_000076 | 3.77 | 0.68 |
| paternally expressed 10 | AB028974 | NM_015068 | 3.54 | 0.95 |
| thromboxane A2 receptor | D38081 | NM_001060 | 2.32 | 0.13 |
| B-myb | X13293 | NM_002466 | 2.11 | 0.28 |
| retinoblastoma-associated protein HEC | AF017790 | NM_006101 | 1.86 | 0.13 |
| cholesterol 25-hydroxylase | AF059214 | NM_003956 | 1.86 | 0.56 |
| G protein-coupled receptor, family C, group 5, member B | AC004131 | | 1.83 | 0.32 |
| thymidine kinase 1 | M15205 | NM_003258 | 1.80 | 0.39 |
| CREM (cAMP responsive element modulator) | S68134 | NM_001881 | 1.78 | 0.30 |
| alpha-actinin-2-associated LIM protein | AF002282 | NM_014476 | 1.77 | 0.42 |
| desmoplakin (DPI, DPII) | AL031058 | | 1.74 | 1.03 |
| MCM6 minichromosome maintenance deficient 6 | D84557 | NM_005915 | 1.72 | 0.06 |
| erythrocyte membrane protein band 4.9 (dematin) | U28389 | NM_001978 | 1.71 | 0.25 |
| GTP cyclohydrolase 1 | U19523 | NM_000161 | 1.61 | 0.04 |
| KIAA0186 gene product | D80008 | NM_021067 | 1.47 | 0.11 |
| cell division cycle 2 protein | X05360 | NM_001786 | 1.35 | 0.43 |
| hypothetical protein from clone 643 | AF091087 | NM_020467 | 1.25 | 0.22 |
| ubiquitin carrier protein E2-C | U73379 | NM_007019 | 1.23 | 0.12 |
| mitotic checkpoint kinase Mad3L | AF053306 | NM_001211 | 1.22 | 0.47 |
| V-Erba Related Ear-3 Protein | HG3510-HT3704 | | 1.20 | 0.20 |
| glycogen phosphorylase (PYGL) | AF046798 | | 1.16 | 0.54 |
| fms-related tyrosine kinase 4, VEGFR-3 | X69878 | NM_002020 | 1.10 | 0.00 |
| BTB (POZ) domain containing 3 | AB023169 | NM_014962 | 1.10 | 0.08 |
| SMC4 structural maintenance of chromosomes 4-like 1 | AB019987 | NM_005496 | 1.09 | 0.59 |
| (yeast) | | | | |
| high-mobility group protein 2 | X62534 | NM_002129 | 1.07 | 0.04 |
| alpha topoisomerase | L47276 | | 1.04 | 0.49 |
| BEC-specific suppressed by AdProx-1 (63 genes) | | | | |
| neuropilin-1 | AF016050 | NM_003873 | −3.99 | 0.42 |
| ras-related C3 botulinum toxin substrate 2, RAC2 | M64595 | NM_002872 | −3.87 | 0.47 |
| tripartite motif-containing 22 | X82200 | NM_006074 | −3.56 | 0.28 |
| small inducible cytokine A2 (monocyte chemotactic protein | M26683 | NM_002982 | −3.56 | 0.03 |
| 1) | | | | |
| zinc finger protein 238 | AJ223321 | NM_006352 | −3.08 | 0.13 |
| uPA | X02419 | | −3.05 | 0.02 |
| transcription factor EC | D43945 | NM_012252 | −3.04 | 0.08 |
| RNase A, pancreatic | D26129 | NM_002933 | −2.72 | 0.02 |
| vitamin A responsive; cytoskeleton regulated | AF070523 | NM_006407 | −2.51 | 0.6 |
| interleukin 6 | X04430 | NM_000600 | −2.42 | 0.63 |
| Rho GDP dissociation inhibitor (GDI) beta | X69549 | NM_001175 | −2.42 | 0.03 |
| matrix metalloproteinase 14 | X83535 | NM_004995 | −2.37 | 0.08 |
| E3 ubiquitin ligase SMURF2 | AA630312 | NM_022739 | −2.22 | 0.06 |
| death receptor 6 | AF068868 | NM_014452 | −2.16 | 0.61 |
| protein C receptor, endothelial (EPCR) | L35545 | NM_006404 | −2.09 | 0.14 |
| hematopoietic and neural membrane protein (HNMP-1) | U87947 | NM_001425 | −2.08 | 0.63 |
| KIAA0836 | AB020643 | | −2.07 | 0.44 |
| chondroitin sulfate proteoglycan 2 (versican) | X15998 | NM_004385 | −1.99 | 0.65 |
| regulator of G-protein signaling 4 | AI267373 | NM_005613 | −1.93 | 0.54 |
| phosphofructokinase, muscle | U24183 | NM_000289 | −1.93 | 0.11 |
| IGF-II mRNA-binding protein 3 | U97188 | NM_006547 | −1.9 | 0.23 |
| neuronal cell adhesion molecule Nr-CAM/hBRAVO | AB002341 | NM_005010 | −1.89 | 0.13 |
| cell surface glycoprotein CD44 | L05424 | | −1.84 | 0.12 |
| plasminogen activator inhibitor-1 | J03764 | NM_000602 | −1.83 | 0.33 |
| AF1Q protein | U16954 | NM_006818 | −1.79 | 0.23 |
| Homo sapiens clone 24674 mRNA sequence | AF070578 | | −1.76 | 0.01 |
| nicotinamide N-methyltransferase | U08021 | NM_006169 | −1.74 | 0.49 |
| lactate dehydrogenase B | X13794 | | −1.73 | 0.08 |
| KIAA0537 gene product | AB011109 | NM_014840 | −1.73 | 0.08 |
| LIM domain protein | X93510 | NM_003687 | −1.67 | 0.11 |
| lymphocyte antigen 75, DEC-205 | AF011333 | NM_002349 | −1.61 | 0.08 |
| natural killer cell transcript 4 | AA631972 | NM_004221 | −1.59 | 0.05 |
| phospholipase A2 | M72393 | | −1.58 | 0.41 |
| R-ras | M14949 | | −1.56 | 0.1 |
| adenylyl cyclase-associated protein 2 | N90755 | NM_006366 | −1.55 | 0.08 |
| leupaxin | AF062075 | NM_004811 | −1.53 | 0.3 |
| signal transducer and activator of transcription 6 (STAT6) | AF067575 | | −1.51 | 0.45 |
| LYL-1 | M22637 | | −1.51 | 0.14 |
| selectin P | M25322 | NM_003005 | −1.47 | 0.37 |
| protein kinase, cAMP-dependent, catalytic, beta | M34181 | NM_002731 | −1.43 | 0.49 |
| TRAM-like protein | D31762 | NM_012288 | −1.42 | 0.43 |
| guanylate binding protein 2, interferon-inducible | M55543 | NM_004120 | −1.41 | 0.51 |
| intercellular adhesion molecule 2 | X15606 | NM_000873 | −1.38 | 0.13 |
| proteoglycan 1, secretory granule | X17042 | NM_002727 | −1.35 | 0.47 |
| tropomyosin 1 (alpha) | Z24727 | NM_000366 | −1.32 | 0.1 |
| fibroblast activation protein, alpha subunit | U09278 | NM_004460 | −1.25 | 0.12 |
| hypothetical protein DKFZp564D0462 | AL033377 | | −1.25 | 0.23 |
| mitogen-activated protein kinase-activated protein kinase 3 | U09578 | NM_004635 | −1.2 | 0.35 |
| amyloid beta (A4) precursor protein-binding | U62325 | | −1.2 | 0.18 |
| AXL receptor tyrosine kinase | M76125 | NM_001699 | −1.19 | 0.3 |
| integrin alpha 5 | X06256 | NM_002205 | −1.18 | 0.02 |
| prion protein (PrP) | U29185 | | −1.18 | 0.07 |
| TRAF family member-associated NFKB activator | U59863 | NM_004180 | −1.17 | 0.13 |
| annexin VI | Y00097 | NM_001155 | −1.16 | 0.12 |
| transcobalamin II | L02648 | NM_000355 | −1.16 | 0.12 |
| sushi-repeat-containing protein, X chromosome | U61374 | NM_006307 | −1.13 | 0.09 |
| bone morphogenetic protein 6 | M60315 | NM_001718 | −1.13 | 0.39 |
| hypothetical protein from clones 23549 and 23762 | U90908 | NM_021226 | −1.1 | 0.6 |
| retina cDNA randomly primed sublibrary, EST | W28438 | | −1.09 | 0.36 |
| TU3A protein | AF035283 | | −1.06 | 0.29 |
| keratin 7 | AJ238246 | NM_005556 | −1.05 | 0.53 |
| latent transforming growth factor beta binding protein 2 | Z37976 | NM_000428 | −1.04 | 0.13 |
| N-cadherin | M34064 | NM_001792 | −1.02 | 0.12 |
| cDNA DKFZp564J0323 (from clone DKFZp564J0323) | AL049957 | | −1.01 | 0.22 |

¹The change is expressed as the log₂ ratio.
²Standard deviation of the change in the expression level.

The ability of recombinant Prox-1 expression in BECs (where it is normally absent) to modify the transcriptional program of these cells towards the lymphatic endothlial cell phenotype was also investigated. The control, AdLacZ, did not significantly alter the expression of BEC- or LEC-specific transcripts as determined by oligonucleotide microarray analyses. By contrast, AdProx-1 increased expression of many LEC-specific mRNAs, such as VEGFR-3, p57Kip2, desmoplakin I/II and alpha-actinin-associated LIM protein (see Table 8). Suprisingly, Prox-1 also suppressed the expression of about 40% of genes characteristically expressed in BECs, such as the transcription factor STAT6, the UFO/axl receptor tyrosine kinase, neuropilin-1 (NRP-1), monocyte chemoattractant protein-1 (MCP-1) and integrin α5 (see Table 7 and Table 8). These gene expression results are in agreement with the in vivo studies of lymphatic vessels. For example, VEGFR-3 and desmoplakin I/II are found in the lymphatic endothelium (Ebata et al., Microvasc. Res. 61:40-48. 2001; Kaipainen et al., Proc. Natl. Acad. Sci. U.S.A. 92: 3566-70. 1995), and the VEGR co-receptor NRP-1, which was suppressed by Prox-1 in the BECs, was found to be expressed in blood vessels, but not in lymphatic vessels in mouse skin.

TABLE 8
Examples of LEC- and BEC-specific genes regulated by Prox-1

| | LEC-specific, up-regulated | BEC-specific, down-regulated |
|---|---|---|
| Adhesion molecules | | Integrin alpha 5 |
| | | ICAM-2 |
| | | CD44 |
| | | Nr-CAM |
| | | P-selectin |
| Cytoskeletal proteins | Desmoplakin I and II | leupaxin |
| | alpha-actinin-2 associated LIM | |
| | protein | |
| ECM proteins | | versican |
| | | proteoglycan 1 |
| ECM proteins | | versican |
| | | proteoglycan 1 |
| ECM modulation | | MMP-14 |
| | | uPA |
| | | PAI-I |
| Receptor tyrosine kinases | VEGFR-3 | UFO/axl |
| Transcription factors | CREM | STAT6 |
| | ear-3 | |
| Cytokines, chemokines | | TFEC |
| and receptors | | IL-6 |
| | | MCP-1 |
| Cell cycle control | p57Kip2 | |
| | cyclin E2 | |
| Other | cholesterol 25-hydroxylase | Neuropilin-1 |
| | thromboxane A2 receptor | endothelial cell protein C |
| | | receptor |
| Total | 28 genes | 63 genes |
| | (19% of LEC-specific genes) | (38% of BEC-specific genes) |

Genes shown in bold were confirmed by Northern blotting or RT-PCR.

In order to determine whether the Prox-1-induced changes in gene expression were cell-type specific, changes in gene expression after AdProx-1 or AdLacZ infection were analyzed in additional endothelial cell types, i.e., coronary artery endothelial cells (CAECS) and saphenous vein endothelial cells (SAVECs), as well as a non-endothelial cell type, i.e., amniotic epithelial cells (AEC). In all of these cell types, AdProx-1 strongly up-regulated Cyclins E1 and E2, Histone H2B, and PCNA. However, AdProx-1 induced VEGFR-3 expression only in CAECs and SAVECs, and not in AECs.

These results are consistent with the lack of lymphatic differentiation in Prox-1-deficient embryos. Interestingly, the expression of Prox-1 in primary endothelial cells leads to up-regulation of VEGFR-3 receptor tyrosine kinase, which is specific for the lymphatic endothelium after midgestation and is essential for proper lymphatic growth and function (Karkkainen and Petrova, Oncogene 19:5598-5605. 2000). For example, inactivating mutations of VEGFR-3 in humans and mice lead to lymphatic hypoplasia and lymphedema (Jeltsch et al., Science 276:1423-1425. 1997; Karkkainen et al., Nat. Genet., 25:153-159. 2000; Karkkainen et al, Trends Mol. Med. 7:18-22. 2001). The results described above therefore suggest that the up-regulation of VEGFR-3 expression by Prox-1 is one of the key pathways involved in the establishment of lymphatic endothelial cell identity and also suggest that the distinct phenotypes of cells in the adult vascular endothelium are plastic and sensitive to transcriptional reprogramming, which is useful in the therapeutic methods of the invention affecting endothelial cells.

### Example 5

### Ex-vivo Cell Stimulation and Gene Therapy for Lymphedema with AdProx-1 Transfected Cells

The ability of Prox-1 to regulate genes specifically involved in LEC development provides a means for treatment of individuals exhibiting a LEC disorder or condition resulting from either an increase or decrease in LEC gene expression levels. Prox-1 upregulation is useful in promoting LEC development as a treatment for LEC disorders characterized by an under-developed lymphatic system of a condition characterized by a risk of wider-development such as lymphedema. Conversely, Prox-1 inhibition is useful in downregulating LEC development as a treatment for LEC disorders characterized by an over-developed lymphatic system such as lymphedema. It is known in the art that ex vivo transfection of cells and subsequent transfer of these cells to patients is an effective method to upregulate in vivo levels of the specific gene transferred and to provide relief from a disease resulting from under-expression of the gene(s) (Gelse et al., Arthritis Rheum. 48:430-41. 2003; Huard et al, Gene Ther 9:1617-26. 2002; Kim et al., Mol. Ther. 6:591-600 2002).

To develop a therapy for treating irregularities of LEC development, endothelial cells, such as CAECs, SAVECs, LECs or BECs, are isolated from individuals experiencing an LEC disorder (e.g. lymphedema) and then placed in an appropriate culture medium (see above) to promote the growth and viability of the cells. The cells are then transfected as described with the AdProx-1 vector as described above to initiate LEC differentiation of the non-LECs in vitro and to promote growth of the LECs in culture. These transfected cells are then transferred into an affected patient in therapeutically effective numbers to promote LEC expansion in vivo. In preferred embodiments, the manipulated cells are autologous cells. These cells are delivered by one or more administrations typically involving injection. The cells are delivered at a local site of an LEC disease or disorder such as lymphedema or systemically.

Addition of the Prox-1 transfected cells to patients with lymphedema provides supplementary LECs that are incorporated into the lymphatic network to promote lymphatic development and effectuate lymph clearance to relieve the symptoms of lymphedema. It is contemplated that a method comprising AdProx-1 transfection into endothelial cells and administration of transfected cells is useful in the treatment of any disease characterized by an alteration in LEC numbers or activity, such as lymphedema, lymphangioma, lymphangiomyeloma, lymphangiomatosis, lymphangiectasis, lymphosarcoma, and lymphangiosclerosis. Additionally, such methods are useful in ameliorating a symptom (e.g., lymph-induced swelling in the case of lymphedema) associated with such diseases.

### Example 6

### Characterization of LEC-Specific Genes

LEC-specific genes were further analyzed using a subtraction library between the LEC and BEC genes. To construct the library, total RNA was isolated as previously described and 5 μg of total RNA was pre-amplified using a SMART™ PCR cDNA synthesis kit (BD Biosciences Clontech). After RsaI-digestion, PCR-Select cDNA subtraction was carried out in both directions, resulting in selective amplification of differentially expressed sequences, and subtracted LEC and BEC cDNA libraries were prepared (BD Biosciences Clontech). Subtractive hybridization was performed with a 1 (tester): 30 (driver) ratio in both directions and subtracted CDNA pools were amplified by PCR. Forty ng of the purified PCR-amplified product were cloned into the pAtlas vector (PUC-based vector) for the construction of subtracted libraries, although a number of other vectors could be used in the construction, as would be known in the art.

Differential screening of the subtracted LEC-specific library was carried out as described in the PCR-Select Differential Screening Kit User Manual (BD Biosciences Clontech). The LEC-specific subtracted library was plated and individual bacterial clones were picked and grown. After DNA extraction, the inserts were amplified by PCR and used for sequencing. An aliquot of each PCR-amplified insert was also arrayed onto a nylon membrane and used for hybridization with ³²P-labeled cDNA probes. The results from the hybridizations with subtracted LEC-specific (tester) and subtracted BEC-specific (driver) cDNA probes were used for the differential expression analyses.

BLAST (The Basic Local Alignment Search Tool) was used to compare the sequences against nucleotide, protein and EST sequence databases. For unknown sequences, EST contigs were searched and open reading frames were predicted using ORF finder. Protein domain architectures were analyzed using Pfam (Protein families database of alignments and HMMs) and Smart (Simple Modular Architecture Research Tool).

The nucleotide sequences of clones that were differentially expressed in LECs versus BECs were analyzed in the manner described above. Several of the EST or unknown gene fragments detected in the first screen have been investigated further to determine their sequence similarities to known gene sequences and to identify any open reading frames and functional domain similarities. The results are collected in Table 9.

TABLE 9

| Clone | | | | |
|---|---|---|---|---|
| designation | | Human Genome | KIAA Designation # | Expected Gene |
| and (SEQ ID NO) | EST | Accession # | and (SEQ ID NO) | Function |
| LE000100001_A06 | AB014526 | NM_021647 | KIAA0626 | Ig domain motif, |
| (SEQ ID NO: 61) | | | SEQ ID NO: 14 | likely cell adhes [symbol] |
| | | | | function |
| LE0000100050_A01 | AB014544 | NM_014817 | KIAA0644 | Leucine rich moti [symbol] |
| (SEQ ID NO: 59) | | | SEQ ID NO: 15 | cell adhesion |
| LE0000100055_H05 | | | | activity |
| | AI333655 | XM_059074 | no KIAA, | Leucine rich |
| | | | designated hLyrp | repeats, cell |
| | | | SEQ ID NO: 16 | adhesion protein |
| | AI681538 | NM_016647 | SEQ ID NO: 17 | Similar to |
| | | | | mesenchymal stem |
| | | | | cell protein |
| | AA447940 | XM_084655 | SEQ ID NO: 45 | similar to layili |
| | | | | likely cell adhes |
| | | | | function |
| LE000100017_C02 | XM_046677 | D31887 | KIAA0062 | Zinc transporter |
| (SEQ ID NO: 55) | | | SEQ ID NO: 47 | motif, metal ion |
| | | | | transport |
| LE0000100049_E10 | XM_047672 | XM_047672 | KIAA1673 | RNA-binding regio |
| LE0000100054_F09 | | | SEQ ID NO: 26 | similar to RNA |
| LE0000100056_F07 | | | | binding protein |
| SEQ ID NOs: | | | | |
| LE0000100053_A06 | AI761647 | NM_015147 | KIAA0582 | |
| SEQ ID NO: 56 | | | SEQ ID NO: 49 | |
| LE0000100055_G10 | D14657 | NM_014736 | KIAA0101 | |
| LE0000100046_C12 | | | SEQ ID NO: 51 | |
| SEQ ID NOs: 57-58 | | | | |

Several of the LEC-specific genes have been found to correspond to KIAA gene sequences, which are large nucleotide EST clones encoding unknown human proteins. (Kazusa DNA Research Institute, 1532-3, Yana Kisarazu, Chiba, 292-0812, Japan). These LEC-specific genes were further analyzed in several available databases to determine the existence of species homologs and the percent similarity in these homologs and also to reveal amino acid sequences that demonstrate similarity to conserved protein domains.

Analyses of the LEC clone sequences was performed using the HomoloGene database maintained by the U.S. National Center for Biotechnology Information offered by the National Institutes of Health to determine species homologs and orthologs and their percent similarity to the newly isolated human LEC-specific genes. Analyses of the sequences was performed using a resource of curated and calculated homologs for genes as represented by UniGene or by annotation of genomic sequences, generally comparing EST and mRNA sequences from UniGene, as well as transcripts extracted from annotated genomic sequences. (Zhang, et al., J. Comp. Biol. 7:203-14. 2000). The best match for a nucleotide sequence in one organism to a nucleotide sequence in a second organism is based on the degree of similarity between the two sequences, with a minimum alignment of 100 base pairs. The similarity between the two sequences was determined by an alignment score. The alignment score for a sequence pair is the sun of the similarity scores of the sections of the two sequences that aligned.

HomoloGene analyses indicate that human LEC genes corresponding to KIAA0626, KIAA0644, and KIAA0062, are homologous to EST and unknown gene sequences in mouse (all), rat (KIAA0062, KIAA0644), cow (KIAA0062), pig (KIAA0626, KIAA0644) and Xenopus (KIAA0644). The clones showed approximately 80% (±3%) similarity to the genes identified as homologs by HomoloGene, with KIAA0644 demonstrating as high as 86% homology to pig EST sequence BE233028.1 and as low as 72% similarity to an X. laevis gene.

Analyses of the LEC genes using Pfam comparison revealed that nucleotide sequences corresponding to KIAA0626 (SEQ ID NO: 14), KIAA0644 (SEQ ID NO: 15), hLyrp (SEQ ID NO: 16), XM_{—}084655 (SEQ ID NO: 45) and KIAA0062 (SEQ ID NO: 47), showed nucleotide sequence motifs characteristic of encoded transmembrane domains, indicating that the corresponding polypeptides (whose amino acid sequences are set out in SEQ ID NOS: 31, 32, 33, 46 and 48, respectively) are expressed on the cell surface. KIAA1673, KIAA0582 and KIAA0101 do not demonstrate an apparent transmembrane domain and are expected to be cytoplasmic or nuclear proteins. Tissue expression assayed by Northern blot reveals that KIAA0101 is detectable in kidney, thymus, colon and small intestine while KIAA0582 is expressed strongly in heart, skeletal muscle, and ovary, less in kidney and placenta, and more weakly in brain, lung, thymus, small intestine and prostate.

Northern blot analysis of the KIAA0626 transcript indicates that KIAA0626 is expressed specifically in LEC and is found in heart, skeletal muscle and kidney. In situ analysis demonstrates KIAA0626 expression in mouse embryonic day 11 (E11) embryos in the intersomitic tissue and pericytes surrounding the blood vessels, and in the yolk sac vessels, endothelial cells and in the surrounding pericytes. The polynucleotide sequence of KIAA0626 (SEQ ID NO: 14) encodes a 409 amino acid (409 aa) protein (SEQ ID NO: 31) possessing a signal sequence (at amino acids 1-29), an Ig superfamily domain (approximately aa 61-127), a short transmembrane region ( about aa 153-175) and a long 234-amino-acid cytoplasmic domain from about amino acids 176-409. The presence of an Ig domain is expected to assist in binding of the protein to its ligand while the long cytoplasmic domain indicates that KIAA0626 may be involved in intracellular signaling in LECs.

KIAA0644 (SEQ ID NO: 15) is detected by Northern blot analysis primarily in heart and brain tissue. In situ assay of E10 mouse embryos shows KIAA0644 expression throughout the embryo. The KIAA0644 polynucleotide encodes a 811-amino-acid polypeptide (SEQ ID NO: 32) demonstrating a total of 13 leucine rich regions. Leucine-rich regions comprise a short sequence motif of approximately 20-28 amino acids which are present in proteins functioning as cell-adhesion and receptor molecules. Leucine-rich regions, designated below as LRRNT and LRRCT are often flanked by cysteine-rich domains. The KIAA0644 protein contains a leucine-rich N-terminal region (LRRNT: aa 26-54), 11 internal leucine-rich regions (LRR1: aa84-107, LRR2: aa108-131, LRR3: aa132-155, LRR4: aa156-179, LRR5: aa180-203, LRR6: aa204-223, LRR7: aa230-253, LRR8: aa254-277, LRR9: aa278-301, LRR10: aa302-325, and LRR11: aa326-349) and a C-terminal leucine-rich region (LRRCT) from about amino acids 359-404. The KIAA0644 transmembrane domain spans approximately amino acids 696-718, leaving a cytoplasmic domain of about 95 amino acids, from aa719-8 11. The leucine-rich regions of the KIAA0644 gene implicate it in protein-protein interactions characteristic of cell-adhesion or ligand binding.

The hLyrp (SEQ ID NO: 16) mRNA is detectable in skeletal muscle tissue and is localized by in situ hybridization to the lymphatic vessels when compared to Prox-1 staining in E11 and yolk sac of mouse embryos. Similar to KIAA0644, the hLyrp protein (SEQ ID NO: 33) contains a series of leucine-rich regions beginning at the leucine-rich N-terminal region (LRRNT: aa27-55) extending through 5 internal leucine-rich regions (LRR1: aa57-80, LRR2: aa81-104, LRR3: aa105-128, LRR4: aa129-153, LRR5: aa154-176) and ending with a C-terminal leucine-rich region (LRRCT) from approximately aa186-240. The hLyrp polypeptide also contains a transmembrane domain from amino acids 249-272, leaving a short cytoplasmic domain of 22 amino acids. The presence of several consecutive-leucine-rich regions in the hLyrp polypeptide indicates that the polypeptide functions as a cell-adhesion molecule and/or a cell surface receptor.

Several additional sequences shown in Table 3 were isolated with full-length mRNA sequences which are expressed specifically in LECs. Domain prediction of these sequences indicates that KIAA0711 (SEQ ID NO: 81 and 82) contains a BPB/POZ domain spanning approximately amino acids 171-269, this domain is expected to function in protein-protein interactions. POZ domains appear in transcriptional co-factors such as zinc-finger proteins that mediate transcriptional repression and interact with components of histone deacetylase complexes. KIAA0711 also has three Kelch repeats, spanning amino acids 386-437, 439-480, and 484-525, and Kelch motifs have been implicated in the formation of beta sheet structures. Additionally, KIAA0711 mRNA is expressed in a variety of tissues. From highest expression levels to lowest, KIAA0711 mRNA is found in brain and kidney; liver; spleen; lung; ovary, pancreas and heart; smooth muscle and testis. Because this expression pattern was obtained from a single run of RT-PCR ELISA, the expression profile has a chance to include significant run-to-run variations. Accordingly, the expression profiles are most suitable for screening genes for tissue-specific expression on a qualitative level. If more accurate quantitative expression profiles are required, more statistically reliable approaches should be employed (e.g., multiple RT-PCR-ELISA measurements, DNA chip analyses, RNA blot analyses, and the like).

Domain mapping of the sequence corresponding to cDNA DKFZp5640222 (SEQ ID NO: 93) indicates the presence of an N-terminal signal peptide (amino acids 1-23), two internal repeat domains and:an olfactomedin domain (amino acids 361-616), which is detected in proteins such as myocilin, pancortin, and latrophilin. Mutations in the OLF domain-of myocilin are associated with glaucoma

Domain mapping of KIAA1233 (SEQ ID NO: 111) indicates that the KIAA sequence contains six thrombospondin type I repeats, which are found in extracellular matrix proteins and are implicated generally in cell-cell interactions, and more specifically in the complement pathway, in the inhibition of angiogenesis, and in apoptosis. KIAA1233 also contains three immnunoglobulin C-2 type domains, similar to many glycoproteins. Proteins possessing both thrombospondin repeats and immunoglobulin domains are also involved in intracellular interactions, such as cell-adhesion and apoptosis. From highest expression levels to lowest, KIAA1233 mRNA is found in the spinal cord; heart, general brain, lung, liver, kidney, pancreas, various regions of the brain (amygdala, corpus callosum, caudate nucleus, hippocampus, substantia nigra, thalamus, and subthalamic nucleus) and fetal liver; fetal brain; spleen; and testis.

The KIAA0846 (SEQ ID NO: 188) protein contains motifs found in guanine nucleotide exchange factors and is thus probably an intracellular protein, perhaps a signaling protein. KIAA0846 also exhibits two EF-hand motifs found in signalling proteins (e.g. calmodulin, S100B), which undergo a calcium-dependent conformational change and are also found in buffering/transport proteins. From highest expression levels to lowest, KIAA0846 mRNA is found in kidney; heart, brain and lung; liver, spleen and ovary; pancreas, smooth muscle and testis.

Protein FLJ13110 (SEQ ID NOS: 207 and 208) exhibits a TB2/DP1, HVA22 family protein domain and two short transmembrane regions (amino acids 4-22 and 43-65 of SEQ ID NO: 207). The HVA22 family includes members from a wide variety of eukaryotes, including the TB2/DP1 (deleted in severe familial adenomatous polyposis) protein which is deleted in severe forms of familial adenomatous polyposis, an autosomal dominant oncological inherited disease.

The LEC-specific gene screen also identified protein KIAA0937 (SEQ ID NOS: 211 and 212). KIAA0937 contains WWE domains (from approximately amino acids 30-112, and 113-189 of SEQ ID NO: 211) which is named after three of its conserved residues and is predicted to mediate specific protein-protein interactions in ubiquitin and ADP ribose conjugation systems. KIAA0937 is also predicted to contain a zinc finger domain (from amino acids 443-501 of SEQ ID NO: 211) and is expected to be an intracellular transcription factor. From highest expression levels to lowest, KIAA0937 mRNA is found in the spinal cord; the subthalanic nucleus and cerebellum of the brain; the brain in general (including the amygdale, corpus callosum and fetal brain) and ovary; fetal liver, heart, lung, kidney, spleen and parts of the brain (caudate nucleus and hippocampus); testis and pancreas; and smooth muscle.

KIAA0952 (SEQ ID NO: 241 and 242) contains a Broad-Complex, Tramtrack and a Bric-a-brac domain, also known as a POZ (poxvirus and zinc finger) domain. These domains are known to be protein-protein interaction domains found at the N-termnini of several C2H2-type transcription factors, as well as Shaw-type potassium channels. The known structure of these domains reveals a tightly intertwined dimer formed via interactions between an N-terminal polypeptide strand and helix structures.

The protein designated KIAA0429 (SEQ ID) NOS: 391 and 392) is similar to metastasis suppressor protein and contains an actin-binding WH2 domain from approximately amino acids 467-484, as well as a proline-rich region from amino acids 348-466.

Protein FLJ23403 (amino acid sequence, SEQ ID NO:859; polynucleotide sequence, SEQ ID NO:860) shows approximately 85% homology to an unknown mouse protein (GenBank Acc. No. XM_{—}129000) and contains a series of four transmembrane domains spanning amino acids 44-66, 86-108, 115-137 and 452-474.

Additional LEC-specific, upregulated genes include previously unidentified proteins KIAA0186 (SEQ ID NOS: 221 and 222), KIAA0513 (SEQ ID NOS: 235 and 236) and the protein designated FLJ13910 (SEQ ID NOS: 293 and 294).

The manipulation of lymphatic endothelial-cell-specific molecules is expected to be applicable to treatments of LEC diseases disorders associated with tissue edemas. Without wishing to be bound by theory, manipulation of such molecules is expected to modulate endothelial cell-cell or cell-matrix protein interactions or to affect transendothelial transport thereby altering the state of fluid transport across the lymphatic vessel wall. Further, such molecules provide targets for the delivery of therapeutic compounds, such as growth factors, mitogens, and the like, as well as cytostatic or cytotoxic agents known in the art. These therapeutic compounds are targeted to such cells by associating a therapeutic agent with, e.g., a binding partner (such as an antibody) of the LEC surface marker. The transmembrane proteins identified herein, in particular the leucine-rich proteins, also provide useful targets for modulating cell adhesion events integral to lymph clearance.

### Example 7

### Microarray Analysis to Detect LEC- and Lymph-Related Disorders

The LEC-specific genes identified herein are useful in the detection of LEC in vivo and in determining the extent of the lymphatic vasculature in a sample. The LEC-specific genes are also expected to be useful in diagnosing lymphedema and other LEC-related disorders.

Another aspect of the invention is a composition comprising a plurality of polynucleotide probes for use in detecting gene expression pattern(s) characteristic of particular cell type(s) and for detecting changes in the expression pattern of a particular cell type, e.g., lymphatic endothelial cells. The term “polynucleotide probe” is used herein to refer to any one of the nucleic acid sequences listed in SEQ ID NO: 1-30, 45, 47, 49 and 51, or any fragment thereof or a nucleic acid sequence encoding an amino acid sequence listed in SEQ ID NOS: 31-44, 46, 48, and 50, or a fragment thereof. Preferably, the fragment is at least 10 nucleotides in length; more preferably, it is at least 20 nucleotides in length. Such a composition is employed for the diagnosis and treatment of any condition or disease in which the dysfunction or non-function of lymphatic endothelial cells is implicated or suspected. In one embodiment, the present invention provides a composition comprising a plurality of polynucleotide probes, wherein at least a subset of the polynucleotide probes comprises at least a portion of an expressed gene isolated from a population of LEC-specific genes identified above. Also contemplated is a composition comprising a plurality of polynucleotide probes, with at least a subset of such probes each comprising a unique sequence selected from the group of SEQ ID NOs: 1-30, 45, 47, 49 and 51. Preferably, the composition comprises a subset of at least 3 polynucleotides, each having a different sequence selected from the group of SEQ ID NOs: 1-30, 45, 47, 49 and 51. Also preferred are compositions comprising at least 5, at least 7, at least 9, at least 15, at least 20, or at least 25 distinct polynucleotides having sequences selected from the group of SEQ ID NOs: 1-30, 45, 47, 49 and 51.

The composition is particularly useful as a set of hybridizable array elements in a microarray for monitoring the expression of a plurality of target polynucleotides. The microarray comprises a substrate and the hybridizable array elements. The microarray is used, for example, in the diagnosis and prognosis of a disease derived from aberrant lymphatic endothelial cell activity, such as lymphedema, lymphangioma, lymphangiomyeloma, lymphangiomatosis, lymphangiectasis, lymphosarcoma, and lymphangiosclerosis. Compositions may be useful in identifying more than one cell type and may be useful in the diagnosis and prognosis of more than one disease, disorder or condition. Further, useful information is obtained from those probes yielding a signal and from those probes not yielding a signal.

A polynucleotide comprising the sequence of any one of SEQ ID NOS: 1-30, 45, 47, 49 and 51 may be used for the diagnosis of conditions or diseases with which the abnormal expression of any one of the genes encoded by SEQ ID NOS: 1-30, 45, 47, 49 and 51 is associated. For example, a polynucleotide comprising any one of the sequences set forth in SEQ ID NOS: 1-30, 45, 47, 49 and 51 may be used in hybridization or PCR assays of fluids or tissues (e.g.,.obtained from biopsies) to detect abnormal gene expression in patients with lymphedema or another lymph-associated disease. In addition, a polynucleotide comprising a sequence encoding any of the amino acid sequences set forth in SEQ ID NOS: 31-44, 46, 48 or 50 is useful for the diagnosis of conditions or diseases associated with aberrant expression of a polypeptide having any one of those amino acid sequences. Fragments comprising at least 10 nucleotides are also useful in these diagnostic methods.

Expression profiles may be generated using the compositions of the invention comprising SEQ ID NOs: 1-30, 45, 47, 49 and 51. The expression profile generated from the microarray is used to detect changes in the expression of genes implicated in disease.

### Example 8

### Transcription Factors in BECs and LECs

Transcription factors preferentially expressed in the LECs included the zinc finger factor c-maf and the MADS-family transcription factor MEF2C (FIG. 1). Targeted mutagenesis of MEF2C leads to embryonic death at E9.5-10 due to defects in the remodeling of the primary vasculature and abnormal endocardiogenesis (Bi, et al., Dev. Biol. 211:255-267. 1999). MEF2C has been reported to bind the transcription factor Sox18 and to potentiate its activity in endothelial cells (Hosking, et al., Biochem. Biophys. Res. Commun. 287:493-500. 2001). Mouse pups with a homozygous mutation in Sox18 that disrupts the MEF2C complex develop chylous ascites in some genetic backgrounds (Pennisi, D., et al., Nat. Genet. 24:434-437. 2000), suggesting that both proteins may be involved in the regulation of lymphatic development. In line with this hypothesis, RT-PCR analysis of MEF2C^{-/-} embryos showed decreased VEGFR-3 expression (Bi, et al., Dev. Biol. supra).

The STAT6 transcription factor, which is activated in response to IL-4, was expressed specifically in the BECs. Consistent with this observation, the results herein show that the IL-4 receptor was expressed preferentially in BECs, as were some of the IL-4 target chemokines and receptors such as MCP-1 and CXCR4. VEGF stimulation and activation of VEGFR-2 is also known to lead to STAT6 phosphorylation and activation in endothelial cells (Bartoli, et al., J. Biol. Chem. 275:33189-33192. 2000). The absence of STAT6 in LECs, therefore, suggests that the downstream signaling pathways of VEGFR-2 differ in BECs and LECs. Expression patterns of other transcription factors are shown in Table 5.

### Example 9

### Sox18 and Hereditary Lymphedema

Expression of the transcription factor MEF2C is upregulated in LECs. Sox18 (SEQ ID NO: 53, and encoding SOX18, SEQ ID NO: 54), which was reported to interact with MEF2C in mice, was also shown to play a potential role in lymphatic endothelial cell development. To investigate the role of Sox18 in human lymphedema, the correlation of human Sox18 mutants with human hereditary lymphedema was investigated.

The SOX proteins, homologs of the family of SRY transcription factors, are ubiquitous transcription factors which contain a putative high-mobility-group (HMG) DNA binding domain. (Wegner, M., Nucl. Acids Res. 27:1409-20. 1999). SOX proteins bind their DNA targets at a heptameric SOX consensus binding sequence [5′-(A/T)(A/T)CAA(A/T)G-3′] (Pennisi et al., Mol. Cell Bio. 20:9331-36. 2000) and generally bind DNA in the minor groove rather than the major groove of the double helix, which results in transcriptional regulation of the target gene. SOX proteins may also be involved in recruiting other DNA binding proteins to a DNA-protein complex, thereby assisting in transcription regulation (Wegner, supra). SOX18 shares homology with both SOX7 and SOX17, all members of the Group F Sox genes.

SOX18 is involved in vascular development and has been localized to the developing cardiovascular system and sites of angiogenic activity. Mice homozygous for the Ragged (Ra) mutation in Sox18 exhibit chylous ascites and edema (Pennisi et al., Nat. Genet. 24:434-37. 2000), similar to the Chy mouse model of lymphedema (Lyon et al., Mouse News Lett. 71: 26. 1984). The mutation in Ra mice has been determined to be a frameshift mutation that causes truncation of the transactivating domain (Pennisi et al., Nat. Genet. 24:434-37. 2000). Sox18 null mice, however, demonstrate only a slight phenotypic change in hair follicle development and show no signs of edema or irregular vascular development (Downes and Koopman, Trends Cardio. Med. 11:318-24. 2001). This phenotype may be due to redundancy among the Group F Sox members, SOX7 and SOX17. These proteins may substitute for SOX18 function in its absence, but cannot overcome a Sox18 dominant negative mutant such as the Ra mutations. Hence, knocking out the entire Group F family may produce a lymphedema phenotype similar to the Ragged mice.

Mouse and human SOX18 are homologous proteins containing a DNA binding HMG-box of approximately 80 amino acids (97% homologous), a transactivating domain which in mouse is about 93 amino acids (90% homologous), and a C-terminal domain (92% homologous) (Downes and Koopman, supra). The human SOX18 HMG-box has been localized to nucleotides 395-598, corresponding to amino acids 84-151. The mouse HMG-box is encoded by nucleotides 320-532, corresponding to amino acids 78-148. The human transactivation domain has not been delineated to date, but one of skill in the art could readily obtain the human transactivating domain using the -homologous mouse sequence, which is found at amino acids 252-346 of mouse SOX18 (Hosking et al., Gene 262:239-47. 2001). Although the human SOX18 protein exhibits similarities to mouse SOX18 at the primary structural level, there is no known association of a human Sox18 mutant with a disease or condition, such as hereditary lymphedema.

Human Sox18 has been mapped to chromosome 20q.13.3 (Stanojcic et al., Biochem. Biophys. Acta. 1492:237-41. 2000). Elucidation of an inheritable mutation at or near this chromosomal location that correlates with hereditary lymphedema is useful in confirming the genetic basis of the disease, in the screening of patients affected by hereditary lymphedema, in the screening of patients for a pre-disposition to develop hereditary or other forms of lymphedema, and also as a basis for target treatment regimens directed to overcoming the inherited mutation.

To determine the linkage of Sox18 with lymphedema, families with inherited lymphedema are identified for the purpose of conducting linkage and positional candidate gene analyses. Family members are considered affected with hereditary lymphedema if they exhibit asymmetry or obvious swelling of one or both legs or if they have received a medical diagnosis of lymphedema or if there are personal or family reports of extremity swelling or asymmetry.

Biological samples are obtained from members of the families to conduct the genetic analyses. DNA is isolated from EDTA-anticoagulated whole blood by the method of Miller et al., (Nucleic Acids Res. 16:1215. 1998), and from cytobrush specimens using the Puregene DNA isolation kit (Gentra Systems, Minneapolis, Minn.). Analysis of the markers used in the genome scan are performed by methods recognized in the art. See Browman et al., Am. J. Hum. Genetic., 63:861-869 (1998); see also the NHLBI Mammalian Genotyping Service.

To explore the potential role of Sox18 in lymphedema, probands from the lymphedema families are screened for variation by direct sequencing of portions of the Sox18 gene. The sequencing strategy uses amplification primers generated based upon the Sox18 cDNA sequence (SEQ ID NO: 53) and information on the genomic organization (intron-exon data, identified domain motifs) of the related Sox genes. Variable positions (single nucleotide polymorphisms) and unique sequence primers are used to amplify sequences flanking each variable site located in the domains used for analysis.

The Sox18 genomic DNA from both the normal and lymphedema affected individuals is sequenced and a map of mutations detected in the Sox18 gene of lymphedema patients as compared to unaffected individuals is generated. Commonly detected mutations in lymphedema patients, such as a conservative or non-conservative nucleotide change, a deletion, or an insertion, indicates that a mutation in that particular nucleotide confers a pre-disposition to developing lymphedema. Analysis of the genomic DNA of the affected individuals will correlate mutations in the Sox18 genomic sequence and lymphedema.

To confirm the correlation of Sox18 mutations and the development of lymphedema, genetic linkage studies are performed, as set out in the method of identifying genetic polymorphisms described in U.S. patent application number US2003026759 and PCT/US99/06133, each of which is incorporated herein by reference.

Two-point linkage analysis is conducted using an autosomal dominant model predicting 80% penetrance in the heterozygous state, 99% penetrance in the homozygous state, and a 1% phenocopy rate. The frequency of the disease allele is set at 1/10,000. Microsatellite marker allele frequencies are calculated by counting founder alleles, with the addition of counts of non-transmitted alleles. Multipoint analysis is carried out using distances from the Location Database provided by the University of Southampton School of Medicine. Multipoint and 2-point analyses are facilitated using the VITESSE (v1.1) program. (O'Connell, and Weeks, Nature Genet., 11:402-408. 1995).

Analysis of the markers used in the genome scan are performed by methods recognized in the art. [See Browman et al., Am. J. Hum. Genetic., 63:861-869 (1998); see also the NHLBI Mammalian Genotyping Service and databases offered by the Center for Molecular Genetics (Marshfield, Wis.). One of skill in the art readily chooses genetic linkage markers identified in chromosome 20 (specifically 20q13.3), where Sox18 has been localized (Stanojcic et al., supra).

Linkage simulation is performed using SLINK (Weeks et al., Am. J. Hum. Genet. 47:A204. 1990) and linkage is analyzed using MSIM (Ott, J., Proc. Nat. Acad. Sci. USA, 86:4175-4178. 1989) to estimate the potential power of two point linkage analysis in the family being assessed. Marker genotypes are simulated for a marker with heterozygosity of 0.875 under a linked (θ=0) and unlinked (θ=0.5) model using the available individuals. The simulation is set such that the power to detect linkage is greater than 90% for a LOD score threshold of Z(θ) 2.0 and the false positive rate is less than 5%.

Mutations that correlate strongly with a heritable lymphedema are expected to be mutations in functional domains of the SOX18 protein, e.g., the HMG-Box domain or the transactivating domain. Exemplary mutations include missense mutations that cause non-conservative substitutions, nucleotide deletions or insertions that cause frameshifts in the Sox18 coding region, in-frame deletions or insertions such as those affecting a functional domain(s), or alterations of control regions affecting the level of Sox18 expression.

Upon identification of the Sox18 lymphedema-correlated mutations, Sox18 mutant expression vectors containing an isolated mutant Sox18 allele is expressed in, e.g., 293T or endothelial cells. The Sox18 mutant DNA can also be integrated into a plasmid useful in the mammalian two-hybrid system, such as pGAL4, to measure SOX18 interaction with its binding partners, such as MEF2C (Hosking et al., Biochem. Biophys. Res. Comm. 287: 493-500. 2001) or to screen for SOX18 binding partners. For example, pGAL4Sox18 vector links the Sox18 gene to the yeast Gal4 DNA binding domain and a transcriptional activator is linked to a SOX18 binding partner in a separate vector. Co-introduction of these vectors into a host cell will result in detectable reporter gene expression resulting from SOX18 interactions with the binding partner or candidate binding partner. The pCMV-BD and pCMV-AD vectors, which contain a GALA DNA binding domain and the NF-κB transcriptional domain, respectively, are useful in this assay (BD Biosciences Clontech) for constructing and expressing gene fusions, with SOX18 binding activity detected using the luciferase reporter system.

In such a di-hybrid assay, a Sox18 lymphedema-correlated mutant that contains a mutation affecting SOX18 binding via the transactivating domain will decrease the amount of luciferase reporter activity, indicating that the Sox18 lymphedema-correlated mutation may result in lymphedema through a defect in its ability to bind its binding partner through its transactivating domain.

A Sox18 allele is also assessed for a mutation in its HMG-box DNA binding domain through several techniques. DNA binding is assessed in a one-hybrid assay in which the DNA sequence bound by SOX18, e.g. 5′-(A/T)(A/T)CAA(A/T)G-3′ and permutations thereof, is placed in front of (i.e., upstream of or 5′ to) a promoter/reporter gene construct similar to the target plasmid in a two-hybrid assay. The reporter assay then detects binding between a SOX18 protein and its putative DNA binding sequence. DNA binding is also assessed using a gel shift assay performed by incubating a purified SOX18 protein with a ³²P end-labeled DNA fragment containing the SOX18 DNA-binding sequence. The reaction products are then analyzed on a non-denaturing polyacrylamide gel to measure the mobility of DNA-bound or free SOX18. The specificity of a SOX18 polypeptide for the putative binding site is established by competition experiments using DNA fragments or oligonucleotides containing a binding site for SOX18 or other unrelated DNA sequences.

Additionally, fluorescence-based assays for detection of DNA/protein binding are used. SOX18 DNA binding is detected by fluorescence measurement of single fluorophores which are bound to either the DNA or protein. In these assays, protein binding is determined by a change in fluorescence intensity or polarization when DNA-protein complexes form. Alternatively, two DNA fragments, each containing half of the protein binding site, are generated. The two double-stranded DNA fragments have complementary single-strand overhangs that comprise part of the protein binding site. One DNA fragment is labeled with a fluorescence donor while the other is labeled with an acceptor, with fluorescence detected only upon fluorescence resonance energy transfer (FRET). Upon protein binding, the overhangs of the two DNA fragments anneal and bring the fluorescence donor and acceptor into proximity, resulting in transfer of the fluorescence energy, which results in detectable fluorescence of the acceptor. See Heyduk, et al., Nat. Biotechnol. 20:171-6. 2002.

Correlation of a mutation in the human Sox18 genome with the risk of developing lymphedema provides another method for diagnosis and/or treatment of individuals affected by hereditary lymphedema. Elucidation of a Sox18 mutation associated with lymphedema allows for the determination of the SOX18 protein activity is disturbed by the mutation, e.g., DNA binding or protein binding, and provides direction for treatment of patients with lymphedema.

Additionally contemplated is the treatment of patients with Sox18-induced lymphedema with a lymphatic growth factor such as VEGF-C and/or VEGF-D to overcome impaired lymphatic vascular development. For example, treatment of VEGFR-3 defective animals with VEGF-C and/or VEGF-D overcomes the inability of VEGFR-3 to signal, thereby promoting lymphangiogenesis and ameliorating symptoms of lymphedema. Sox18-induced lymphedema patients are treated with a therapeutically effective amount of VEGF-C and/or VEGF-D. In an additional embodiment, VEGF-C and/or VEGF-D are administered to the above patients in conjunction with other therapies designed to relieve the symptoms of lymphedema.

### Example 10

VEGF-C and VEGF-D knockout mice demonstrate aberrant vascular development which can be overcome by administration of exogenous VEGF-C and/or VEGF-D polypeptide. To determine if Sox18 transcriptional regulation can overcome this defect due to its potential interaction with, and transcriptional effect on, the VEGFR-3 promoter, VEGF-C or VEGF-D knockout mice are genetically crossed by interbreeding with mice overexpressing Sox18 from a cell-specific-promoter (e.g. K-14 keratin promoter) or a retroviral vector. The effects of Sox18 activity on lymphedema are assessed through measurement of lymphedema and vascular development, as described in Example 10.

Survival of the knockout mice and detection of lymphatic development in the VEGF-C and/or VEGF-D knockout/Sox18-overexpressing mice indicates that Sox18 induces VEGFR-3 signaling and plays a key role in lymphangiogenesis.

VEGF-C overexpressing mice (K-14-VEGF-C Tg) exhibit an extensive network of lymphatic vasculature, are prone to tumor metastasis, and demonstrate upregulated VEGFR-3 expression and symptoms of lymphedema (U.S. Pat. No. 6,361,946). To determine if Sox18 regulates VEGF-C signaling through VEGFR-3, K-14-VEGF-C Tg mice are crossed to animals which express a naturally mutated Sox18 (Ragged mutation) or a laboratory-designed mutant constructed using site-directed mutagenesis and standard knockout techniques known in the art to generate a mutation in either the DNA-binding or transactivating domain of the SOX protein, resulting in a K-14-VEGF-C Tg/Sox18^{-/-} mouse.

Decreased lymphangiogenesis, decreased incidence of tumor metastasis, and decreased levels of VEGFR-3 exhibited by the K-14-VEGF-C TgSox18^{-/-} double mutant animals as compared to the K-14-VEGF-C Tg single mutant animal indicates that the Sox18 molecule interferes with VEGF-C signaling through VEGFR-3 and that inhibition of the VEGFR-3 signaling in the Sox18 mutant downregulates the lymphangiogenic effects of activated VEGFR-3.

Alternatively, K-14-VEGF-C Tg mice are crossed to mice transgenic for a Sox18 allele that is overexpressed (see above) and the effects of Sox18 upregulation are measured. A decrease in lymphangiogenesis, decreased incidence of tumor metastasis, and decreased levels of VEGFR-3 exhibited by the K-14-VEGF-C Tg/Sox18 overexpressing double mutant animals as compared to the K-14-VEGF-C Tg single mutation indicates that Sox18 transcriptional regulation inhibits VEGFR-3 signaling and is likely a factor in negatively regulating lymphangiogenesis.

A result indicating that Sox18 is a negative regulator of lymphangiogenesis provides a method of treating disorders mediated by extensive lymphatic vasculature, such as lymphangiogenesis in tumor development or lymphangiosarcoma, by administration of a vector providing the SOX18 transcription factor in excess thereby preventing the induction of lymphangiogenic signals.

### Example 11

### Sox18 in Lymphatic Development

Lymphatic endothelial cells show a unique development pattern that is highly regulated by several LEC-specific genes such as VEGFR-3 and Prox-1. Sox18, as a DNA binding protein and transcription factor, is expected to be involved in the regulation of these LEC-specific genes, contributing to the elaboration of a LEC cellular fate. Several lines of evidence indicate that Sox18 may be involved in VEGFR-3 transcription regulation: SOX18 binds to the transcription factor MEF2C in mice, both Sox18-mutant and MEF2C-deficient mice exhibit lymphedema symptoms similar to VEGFR-3 mutant mice, and the VEGFR-3 promoter contains a MEF2C binding site (Iljin et al, FASEB J. 15:1028-36. 2001). These observations support a role for SOX18 in lymphatic development.

To analyze the ability of Sox18 to affect the transcription of LEC-specific growth factors, blood vascular endothelial cells are induced to develop into LECs by the addition of an AdProx-1 vector. Sox18 mRNA and protein levels are measured before and after the addition of the Prox-1 vector. Upregulation of Sox18 after the addition of the Prox-1 vector is expected to correlate with the development of lymphatic endothelial cells, indicating that Sox18 is a factor in. LEC differentiation. Alternatively, either the DNA binding or transactivation activity of Sox18 is disrupted via site-directed mutagenesis, thereby resulting in either a dominant negative or inactive SOX18 protein. The plasmid containing the Sox18-disrupted allele is co-transfected into BECs with the AdProx-1 vector to assess LEC development in the presence of a dysfunctional Sox18 gene. Detection of LEC-specific markers such as LYVE-1 and podoplanin are also used in these experiments to measure the ability of Sox18 to modulate lymphatic development. Additionally, mutant Sox18 is also co-transfected with vectors encoding LEC-specific proteins (e.g., VEGFR-3, Prox-1, LYVE-1) into 293T cells and the ability of the mutated Sox18 to regulate the activities of those genes is assessed. For example, signaling in VEGFR-3 co-transfected 293T cells stimulated with VEGF-C in the presence and absence of Sox18 is assessed using a phosphorylation assay.

Development of the lymphatic vasculature can also be evaluated in Sox18 mutant mice, including Ra mice, Sox18 null mice, and Sox18 mice transgenic for a mutation described herein that correlates with a pre-disposition to lymphedema. Transgenic Sox18 mice exhibiting a symptom of lymphedema are engineered to express a mutation in the mouse gene homologous to the human mutation or are engineered to express the human Sox18 gene containing a lymphedema-specific mutation. Development of the vasculature in these animals is analyzed, as set out in U.S. Pat. No. 6,361,946 (see also Kaipainen et al., Proc. Natl. Acad. Sci. (USA), 92:3566-70. 1995), using techniques known in the art, such as in situ hybridization, to detect VEGF-C and/or VEGFR-3 mRNA expression, antibody detection of VEGF-C and/or VEGFR-3 proteins in vivo, and Evan's blue dye detection to determine the extent of LEC development and to visualize effective lymph drainage in vivo.

An increase in VEGFR-3 signaling in a dominant negative Sox18 mutant transfectant indicates that Sox18 expression has a detrimental effect on VEGFR-3-mediated activity. The invention contemplates a therapy to overcome this type of mutation comprising administering to mammal, such as a human patient, a composition comprising a SOX18 inhibitor, such as a dominant negative gene or dominant negative Sox18 ligand which interferes with the ability of SOX18 to interfere with VEGFR-3 signaling. Alternatively, if Sox18 activation promotes VEGFR-3 activity this provides an indication that a therapy for lymphedema comprises a composition which promotes SOX18 transcriptional activity, such as cells given ex-vivo which overexpress Sox18.

### Example 12

### Sox18 Directed Therapy in Lymphedema

Another aspect of the invention is the use of Sox18 to produce cell-based therapeutic compositions, particularly LEC cell-based compositions. In one embodiment, the cells are autologous cells, i.e., cells of the organism (e.g., human patient) receiving treatment for a disease or disorder of the lymphatic system. The invention contemplates elevating the endogenous expression of Sox18, for example by the modification of expression control regions, e.g., promoters, through recombinant techniques such as homologous recombination. Alternatively, the cells are transformed or transfected with an isolated Sox18, e.g., a heterologous Sox18, for heterologous Sox18 expression, either in vivo or ex vivo.

For example, SOX18 interacts with transcription factor MEF2C, with the complex binding to the VEGFR-3 promoter, thereby inducing VEGFR-3 transcription and affecting VEGFR-3 protein expression and signaling levels. It is contemplated that insertion of a Sox18 gene driven by a retroviral or adenoviral vector into an LEC expressing VEGFR-3 will upregulate VEGFR-3-mediated signaling.

These Sox18-expressing cells are then used as a therapeutic composition in the treatment of patients with an LEC disease or disorder, such as hereditary lymphedema or trauma-induced lymphedema These cells are used to treat any disease or condition associated with a decrease in expression of VEGFR-3, such as lymphangioma, lymphangiomyeloma, lymphangiomatosis, lymphangiectasis, lymphosarcoma, and lymphangiosclerosis.

Additionally, a SOX18 polypeptide or polypeptide fragment is administered to a patient experiencing lymphedema to relieve the symptoms of lymphedema. It is contemplated that administration of either a full-length SOX18 polypeptide or a fragment of SOX18, which contains either the DNA binding domain or the transactivating domain, will bind to its cognate binding partner in vivo and promote VEGFR-3 signaling, or will initiate downstream events in the lymphangiogenic process, thus bypassing a defect in VEGFR-3 signaling or VEGF-C ligand binding involved in lymphedema.

In a related aspect, if SOX18 expression inhibits VEGFR-3 signaling via decreased transcription factor binding or DNA binding, it is expected that inhibition of SOX18 will result in a compensatory upregulation of VEGFR-3, ameliorating deleterious symptoms associated with VEGFR-3 under-expression. Administration of antisense therapy specific for the Sox 18 gene in instances where Sox 18 negatively regulates VEGFR-3 activity will inhibit SOX18 activity thereby allowing VEGFR-3-mediated signaling and lymphatic growth. Due to the potential functional redundancy of the Group F SOX proteins (SOX7/17/18), however, it may be necessary to inactivate all three proteins through a mechanism that inhibits the DNA binding activity of all Group F proteins. This is done, e.g., by targeting the DNA binding domain, which is highly homologous among all the proteins. It is contemplated that recombinant SOX7/17/18 proteins expressing a mutated DNA binding domain, when administered as a pharmaceutical composition (containing all three mutant peptides), will inhibit SOX18 downregulation of VEGFR-3 and induce or promote VEGFR-3 signaling activity. From the foregoing it will be appreciated that, although specific embodiments of the invention have been described herein for purposes of illustration, various modifications may be made without deviating from the spirit and scope of the invention.

All of the above U.S. patents, U.S. patent application publications, U.S. patent applications, foreign patents, foreign patent applications and non-patent publications referred to in this specification and/or listed in the [Application Data Sheet] are incorporated herein by reference, in their entirety.

TABLE 3
Lymphatic EC (187 genes)

| | | | | | | Gene expression | | | | |
|---|---|---|---|---|---|---|---|---|---|---|
| | | | | | | analysis | | Confirmed | | |
| | accession | | Affymetrix | Detection¹ | | signal log | | by: | | signal log |
| | numbers | | ID | BEC | LEC | ratio² | s.d.³ | NB | IF | ratio |
| lung type-I cell membrane-associated | AF030428 | NM_006474 | 41870_at | A | P | 7.4 | 0.925 | | + | |
| protein, podoplanin | | | | | | | | | | |
| lung type-I cell membrane-associated | AI660929 | NM_006474 | 41871_at | A | P | 6.1 | 0.150 | | + | |
| protein, podoplanin | | | | | | | | | | |
| cellular retinol-binding protein | M11433 | NM_002899 | 38634_at | A | P | 7.3 | 0.255 | | | |
| macrophage mannose receptor | M93221 | | 36908_at | A | P | 7.1 | 0.682 | | | |
| (MRC1) | | | | | | | | | | |
| transcription factor C-MAF | AF055376 | NM_005360 | 41504_s_at | P | P | 5.1 | 0.522 | | | |
| transcription factor C-MAF | AF055376 | NM_005360 | 41505_r_at | A | P | 3.9 | 0.588 | | | |
| selenoprotein P | Z11793 | NM_005410 | 34363_at | A | P | 5.0 | 0.331 | | | |
| KIAA0466, immunoglobulin | AB007935 | NM_001542 | 38086_at | A | P | 4.9 | 2.028 | | | |
| superfamily, member 3 | | | | | | | | | | |
| type II membrane protein similar to | AB015629 | NM_014257 | 39270_at | A | P | 4.9 | 0.846 | | | |
| HIV gp120-binding C-type lectin, | | | | | | | | | | |
| CD209 antigen-like | | | | | | | | | | |
| KIAA0626 | AB014526 | NM_021647 | 33241_at | A | P | 4.7 | 0.212 | | | |
| KIAA0711 | AB018254 | NM_014867 | 36453_at | A | P | 4.6 | 0.055 | + | | |
| integrin alpha 9 | D25303 | NM_002207 | 1508_at | A | P | 4.3 | 0.712 | + | + | |
| integrin alpha 9 | D25303 | NM_002207 | 35948_at | A | P | 3.6 | 1.086 | + | + | |
| relaxin H2 | X00948 | NM_005059 | 31732_at | A | P | 4.1 | 0.432 | | | |
| KIAA0644 | AB014544 | NM_014817 | 34214_at | A | P | 3.9 | 0.803 | + | | |
| Cdk-inhibitor p57KIP2 (KIP2) | U22398 | NM_000076 | 1787_at | A | P | 3.8 | 2.020 | + | | 3.3 |
| Cdk-inhibitor p57KIP2 (KIP2) | U22398 | NM_000076 | 39545_at | A | P | 3.0 | 0.150 | + | | 1.4 |
| transient receptor potential channel | AJ006276 | NM_004621 | 36365_at | A | P | 3.8 | 0.988 | | | |
| TRPC6 | | | | | | | | | | |
| cDNA DKFZp564O222 (from clone | AL050002 | | 38312_at | A | P | 3.6 | 0.876 | | | |
| DKFZp564O222) | | | | | | | | | | |
| subtilisin-like protein (PACE4), | M80482 | NM_002570 | 32001_s_at | P | P | 3.6 | 0.334 | | | |
| paired basic amino acid cleaving | | | | | | | | | | |
| system 4 | | | | | | | | | | |
| regulator of G-protein signalling 16, | U70426 | NM_002928 | 41779_at | A | P | 3.6 | 0.673 | | | |
| A28-RGS14p | | | | | | | | | | |
| dihydropyrimidinase related protein- | D78012 | NM_001313 | 40272_at | A | P | 3.5 | 1.192 | | | |
| 1, collapsin response mediator | | | | | | | | | | |
| protein 1 | | | | | | | | | | |
| desmoplakin (DPI, DPII) | AL031058 | NM_004415 | 36133_at | A | P | 3.5 | 0.426 | | + | 1.0 |
| pendrin, solute carrier family, | AF030880 | NM_000441 | 36376_at | A | P | 3.3 | 1.156 | | | |
| member 4 | | | | | | | | | | |
| reelin (RELN) | U79716 | NM_005045 | 37530_s_at | P | P | 3.3 | 0.142 | | | |
| integrin, alpha 1 | X68742 | | 120_at | P | P | 3.3 | 0.080 | + | | |
| integrin alpha 1 | X68742 | | 37484_at | M | P | 2.4 | 0.345 | + | | |
| cholesterol 25-hydroxylase | AF059214 | NM_003956 | 32363_at | A | P | 3.3 | 0.137 | | | 1.5 |
| inhibin beta-B-subunit precursor | M31682 | NM_002193 | 38545_at | P | P | 3.2 | 0.056 | | | 3.0 |
| KIAA1233 | AL109724 | | 38856_at | A | P | 3.1 | 1.540 | | | |
| pre-B cell stimulating factor | L36033 | NM_000609 | 33834_at | A | P | 3.0 | 0.860 | RT- | | |
| homologue (SDF1b) | | | | | | | | PCR | | |
| V-Erba Related Ear-3 Protein | HG3510-HT3704 | | 1147_at | P | P | 2.9 | 0.398 | | | 1.7 |
| antigen identified by monoclonal | X05323 | | 37716_at | P | P | 2.9 | 0.283 | + | | 1.0 |
| antibody MRC OX-2 | | | | | | | | | | |
| apolipoprotein D | J02611 | NM_001647 | 36681_at | M | P | 2.9 | 0.150 | + | | |
| TIMP3, tissue inhibitor of matrix | U14394 | NM_000362 | 1035_g_at | A | P | 2.8 | 0.528 | + | | |
| metalloproteinases | | | | | | | | | | |
| TIMP3 | U14394 | NM_000362 | 1034_at | P | P | 1.9 | 0.224 | + | | |
| aldehyde dehydrogenase 1 | K03000 | NM_000689 | 37015_at | P | P | 2.8 | 0.275 | | | |
| prospero-related homeobox 1 (prox | U44060 | NM_002763 | 31918_at | A | P | 2.8 | 0.299 | + | + | (5, 6) |
| 1) | | | | | | | | | | |
| matrix Gla protein | AI953789 | NM_000900 | 36683_at | A | P | 2.6 | 0.250 | + | | |
| neuronal pentraxin II (NPTX2) | U29195 | | 35663_at | A | P | 2.6 | 1.267 | | | 2.9 |
| histatin 2 (HIS2) | M26665 | NM_000200 | 41148_at | A | P | 2.6 | 1.009 | | | |
| ADDL mRNA for adducin-like | D67031 | NM_016824 | 33102_at | P | P | 2.6 | 0.277 | + | | |
| protein, adducin 3 (gamma) | | | | | | | | | | |
| adducin 3 (gamma) | U37122 | NM_016824 | 33103_s_at | M | P | 2.4 | 0.397 | + | | |
| MADS box transcription enhancer | L08895 | NM_002397 | 37710_at | A | P | 2.5 | 0.540 | + | | |
| factor 2, polypeptide C (myocyte | | | | | | | | | | |
| enhancer factor 2C) | | | | | | | | | | |
| MADS box transcription enhancer | | NM_002397 | 37712_g_at | A | P | 1.9 | 0.197 | + | | |
| factor 2, (myocyte enhancer factor | | | | | | | | | | |
| 2C) | | | | | | | | | | |
| MADS box transcription enhancer | S57212 | NM_002397 | 37711_at | A | P | 1.4 | 0.442 | + | | |
| factor 2, polypeptide C (myocyte | | | | | | | | | | |
| enhancer factor 2C) | | | | | | | | | | |
| phosphoglucomutase 5 | L40933 | NM_021965 | 33694_at | A | P | 2.5 | 0.431 | | | |
| cyclin E2 | AF102778 | NM_004702 | 35249_at | A | P | 2.5 | 0.906 | | | 5.8 |
| interleukin 7 (IL7) | M29053 | | 33966_at | A | P | 2.4 | 0.191 | | | |
| interleukin 7 | J04156 | NM_000880 | 1159_at | A | P | 1.9 | 0.921 | | | |
| cDNA DKFZp586L0120 (from clone | AL050154 | | 38351_at | P | P | 2.4 | 0.135 | | | |
| DKFZp586L0120) | | | | | | | | | | |
| peroxisome proliferative activated | L40904 | NM_005037 | 37104_at | A | P | 2.4 | 0.502 | | | |
| receptor, gamma, PPARG | | | | | | | | | | |
| fatty acid binding protein 4 | AA128249 | NM_001442 | 38430_at | P | P | 2.4 | 0.132 | | | |
| protein kinase C zeta | Z15108 | NM_002744 | 362_at | P | P | 2.4 | 0.008 | | | |
| 46 kDa coxsackievirus and | Y07593 | NM_001338 | 37534_at | P | P | 2.3 | 0.137 | | | 2.2 |
| adenovirus receptor (CAR) protein | | | | | | | | | | |
| PAC clone RP4-751H13 from 7q35-qter, | AC004877 | | 39837_s_at | A | P | 2.3 | 0.714 | | | |
| zinc finger-like | | | | | | | | | | |
| thymidine kinase 1, soluble | M15205 | NM_003258 | 910_at | P | P | 2.3 | 0.205 | | | 2.1 |
| thymidine kinase 1 | K02581 | NM_003258 | 41400_at | M | P | 1.7 | 0.193 | | | 1.4 |
| Pig7 (PIG7), LPS-induced TNF-alpha | AF010312 | NM_004862 | 37024_at | A | P | 2.3 | 0.233 | | | 4.6 |
| factor | | | | | | | | | | |
| LPS-induced TNF-alpha factor | AL120815 | NM_004862 | 37025_at | P | P | 1.3 | 0.327 | | | 3.8 |
| lipase A, lysosomal acid, cholesterol | X76488 | NM_000235 | 38745_at | P | P | 2.3 | 0.281 | | | |
| esterase | | | | | | | | | | |
| ubiquitin specific protease 13 | U75362 | NM_003940 | 40701_at | A | P | 2.2 | 0.334 | | | |
| (isopeptidase T-3) | | | | | | | | | | |
| carcinoembryonic antigen-related cell | X16354 | NM_001712 | 988_at | P | P | 2.2 | 0.048 | | | |
| adhesion molecule 1 (biliary | | | | | | | | | | |
| glycoprotein) CEACAM1 | | | | | | | | | | |
| cDNA DKFZp586D0918 (from clone | AL049370 | | 41856_at | P | P | 2.1 | 0.385 | | | |
| DKFZp586D0918) | | | | | | | | | | |
| KIAA0598, B cell RAG associated | AB011170 | NM_014863 | 35350_at | P | P | 2.1 | 0.154 | | | |
| protein | | | | | | | | | | |
| RAMP2 (receptor (calcitonin) | AJ001015 | NM_005854 | 38177_at | P | P | 2.1 | 0.361 | | | |
| activity modifying protein 2) | | | | | | | | | | |
| cholesteryl ester transfer protein | M30185 | NM_000078 | 40741_at | A | P | 2.1 | 0.191 | | | |
| precursor | | | | | | | | | | |
| epithelial membrane protein 2 | U52100 | NM_001424 | 39631_at | P | P | 2.0 | 0.141 | | | 1.0 |
| MHC class II lymphocyte antigen | M83664 | NM_002121 | 38095_i_at | A | P | 2.0 | 1.368 | | | |
| (HLA-DP) beta chain | | | | | | | | | | |
| MHC class II lymphocyte antigen | M83664 | NM_002121 | 38096_f_at | A | P | 1.2 | 0.034 | | | |
| (HLA-DP) beta chain | | | | | | | | | | |
| beta-arrestin 2 | AF106941 | NM_004313 | 33283_at | A | P | 2.0 | 0.273 | | | |
| mitotic checkpoint kinase Bub1 | AF053305 | NM_004336 | 41081_at | A | p | 2.0 | 0.195 | | | |
| (BUB1) | | | | | | | | | | |
| KIAA0229, similar to human ankyrin | D86982 | | 40971_at | P | P | 2.0 | 0.195 | | | 1.6 |
| 1(S08275) | | | | | | | | | | |
| Sprouty 1 homolog (antagonist of | AF041037 | | 38767_at | P | P | 2.0 | 0.209 | | | |
| FGF signaling) | | | | | | | | | | |
| guanine nucleotide exchange factor | | NM_012294 | 38062_at | P | P | 2.0 | 0.497 | | | 2.1 |
| for Rap1; M-Ras-regulated GEF, | | | | | | | | | | |
| KIAA0277 | | | | | | | | | | |
| translin | X78627 | NM_004622 | 36177_at | A | P | 2.0 | 0.140 | | | |
| erythrocyte membrane protein band | U28389 | NM_0019178 | 37192_at | P | P | 2.0 | 0.265 | | | 1.9 |
| 4.9 (dematin) | | | | | | | | | | |
| KIAA0846 protein | AB020653 | NM_015376 | 34748_at | A | P | 2.0 | 0.457 | | | |
| glia maturation factor, gamma | W07033 | NM_004877 | 35261_at | P | P | 1.9 | 0.083 | | | 1.0 |
| insulin-like growth factor binding | X16302 | NM_000597 | 40422_at | A | P | 1.9 | 1.157 | | | |
| protein 2 (IGFBP-2) | | | | | | | | | | |
| smooth muscle myosin heavy chain | S67247 | | 32838_at | A | P | 1.9 | 0.179 | | | |
| isoform Smemb | | | | | | | | | | |
| TTG-2 (cysteine rich protein with | X61118 | NM_005574 | 32184_at | P | P | 1.9 | 0.221 | 1.0 | | |
| LIM motif), LIM domain only 2 | | | | | | | | | | |
| (rhombotin-like 1) | | | | | | | | | | |
| cyclin B2 | AL080146 | NM_004701 | 32263_at | A | P | 1.9 | 0.276 | | | |
| KIAA0353 | AB002351 | | 39544_at | P | P | 1.9 | 0.158 | | | 1.8 |
| KIAA0559, piccolo (presynaptic | AB011131 | | 37780_at | A | P | 1.9 | 0.330 | | | |
| cytomatrix protein) | | | | | | | | | | |
| G protein-coupled receptor, family C, | AC00413 | NM_016235 | 40240_at | P | P | 1.9 | 0.047 | | | 2.1 |
| group 5, member B | | | | | | | | | | |
| G protein-coupled receptor, family C, | AI801872 | NM_016235 | 40239_g_at | P | P | 1.4 | 0.303 | | | |
| group 5, member B | | | | | | | | | | |
| CREM (cyclic AMP-responsive | S68134 | NM_001881 | 32066_g_at | P | P | 1.9 | 0.098 | | | 1.7 |
| element modulator beta isoform) | | | | | | | | | | |
| CREM (cyclic AMP-responsive | S68134 | NM_001881 | 32065_at | P | P | 1.8 | 0.241 | | | 2.0 |
| element modulator beta isoform) | | | | | | | | | | |
| CREM (cyclic AMP-responsive | S68271 | NM_001881 | 32067_at | P | P | 1.5 | 0.182 | | | 2.0 |
| element modulator beta isoform) | | | | | | | | | | |
| hypothetical protein FLJ13110 | AL080222 | NM_022912 | 36096_at | A | P | 1.9 | 0.387 | | | |
| inositol(myo)-1(or 4)- | AF014398 | NM_014214 | 36496_at | A | P | 1.8 | 0.590 | | | 3.2 |
| monophosphatase 2 | | | | | | | | | | |
| KIAA0937 protein | AB023154 | | 35369_at | P | P | 1.8 | 0.185 | | | |
| mitotic spindle coiled-coil related | AF063308 | NM_006461 | 32120_at | A | P | 1.8 | 0.257 | | | |
| protein | | | | | | | | | | |
| cysteine and glycine-rich protein 2 | U57646 | NM_001321 | 41401_at | A | P | 1.8 | 0.431 | | | 4.3 |
| (CSRP2) | | | | | | | | | | |
| topoisomerase (DNA) II alpha | AI375913 | NM_001067 | 40145_at | P | P | 1.8 | 0.239 | | | 1.0 |
| (170 kD) | | | | | | | | | | |
| DNA topoisomerase II | J04088 | NM_001067 | 1592_at | P | P | 1.2 | 0.162 | | | |
| protein phosphatase inhibitor 2 | U68111 | | 33180_at | P | P | 1.8 | 0.319 | | | |
| (PPP1R2) | | | | | | | | | | |
| KIAA0186 | D80008 | NM_021067 | 39677_at | A | P | 1.8 | 0.269 | | | 1.4 |
| dual-specificity tyrosine-(Y)- | Y12735 | NM_003582 | 39931_at | P | P | 1.8 | 0.146 | | | |
| phosphorylation regulated kinase 3 | | | | | | | | | | |
| (Dyrk3) | | | | | | | | | | |
| kinesin-like spindle protein HKSP | U37426 | NM_004523 | 40726_at | M | P | 1.8 | 0.439 | | | |
| (HKSP) | | | | | | | | | | |
| huntingtin-associated protein | U94190 | NM_003947 | 40655_at | P | P | 1.8 | 0.529 | | | 1.4 |
| interacting protein (duo) | | | | | | | | | | |
| diubiquitin | AL031983 | NM_006398 | 39959_at | A | P | 1.8 | 0.841 | | | |
| bikunin, serine protease inhibitor, | U78095 | NM_021102 | 34348_at | A | P | 1.8 | 0.398 | | | |
| Kunitz type, 2 | | | | | | | | | | |
| cytochrome P-450-1 (TCDD- | K03191 | NM_000499 | 36767_at | A | P | 1.7 | 0.165 | | | 2.0 |
| inducible) | | | | | | | | | | |
| cytochrome P(1)-450 | X02612 | NM_000499 | 1025_g_at | P | P | 1.1 | 0.125 | | | 1.7 |
| KIAA0513 | | NM_014732 | 38735_at | A | P | 1.7 | 0.297 | | | |
| protein phosphatase inhibitor 2 | U68111 | | 812_at | P | P | 1.7 | 0.185 | | | |
| (PPP1R2) | | | | | | | | | | |
| RAMP3 (receptor (calcitonin) | AJ001016 | NM_005856 | 35152_at | P | P | 1.7 | 0.228 | | | |
| activity modifying protein 3) | | | | | | | | | | |
| B-myb | X13293 | NM_002466 | 1854_at | M | P | 1.7 | 0.455 | | | 2.3 |
| KIAA0952 | AB023169 | NM_014962 | 37755_at | P | P | 1.7 | 0.254 | | | 1.0 |
| interferon stimulated gene (20 kD), | U88964 | NM_002201 | 33304_at | A | P | 1.7 | 0.178 | | | |
| HEM45 | | | | | | | | | | |
| GS3955 | D87119 | NM_021643 | 717_at | P | P | 1.7 | 0.107 | | | 1.6 |
| GS3955 | D87119 | NM_021643 | 40113_at | P | P | 1.3 | 0.098 | | | 1.3 |
| GRB2-related adaptor protein (Grap) | U52518 | NM_006613 | 805_at | A | P | 1.7 | 0.147 | | | |
| KIAA1071 protein | AB028994 | | 38286_at | A | P | 1.7 | 0.625 | | | |
| RNA-binding protein gene with | D84111 | NM_006867 | 34162_at | P | P | 1.7 | 0.347 | | | |
| multiple splicing, RBP-MS/type 5 | | | | | | | | | | |
| RNA-binding protein gene with | D84111 | NM_006867 | 34163_g_at | P | P | 1.6 | 0.147 | | | |
| multiple splicing, RBP-MS/type 5 | | | | | | | | | | |
| RBP-MS/type 4, RNA-binding protein | D84110 | NM_006867 | 1276_g_at | P | P | 1.5 | 0.263 | | | |
| gene with multiple splicing | | | | | | | | | | |
| RBP-MS/type 4, RNA-binding protein | D84110 | NM_006867 | 38049_g_at | P | P | 1.3 | 0.268 | | | |
| gene with multiple splicing | | | | | | | | | | |
| RBP-MS/type 3, RNA-binding protein | D84109 | NM_006867 | 38047_at | P | P | 1.2 | 0.225 | | | |
| gene with multiple splicing | | | | | | | | | | |
| alpha-actinin-2-associated LIM | AF002282 | NM_014476 | 39690_at | A | P | 1.7 | 0.728 | | | 1.5 |
| protein | | | | | | | | | | |
| semaphorin-III (Hsema-I), | L26081 | NM_006080 | 33461_at | A | P | 1.6 | 0.445 | | | |
| semaphorin 3A | | | | | | | | | | |
| IQ motif containing GTPase | U51903 | NM_006633 | 1647_at | M | P | 1.6 | 0.395 | | | |
| activating protein 2 | | | | | | | | | | |
| Arrestin, Beta 2 | HG2059-HT2114 | | 957_at | P | P | 1.6 | 0.342 | | | |
| retinoblastoma-associated protein | AF017790 | NM_006101 | 40041_at | P | P | 1.6 | 0.153 | | | 2.0 |
| HEC | | | | | | | | | | |
| LIM domain binding protein (LDB1) | AF052389 | NM_001290 | 36065_at | P | P | 1.6 | 0.153 | | | 1.9 |
| dual specificity phosphatase 5 | U15932 | NM_004419 | 529_at | P | P | 1.6 | 0.207 | | | |
| Homo sapiens cDNA 3′, mRNA | AI557322 | | 39611_at | P | P | 1.6 | 0.081 | | | |
| sequence | | | | | | | | | | |
| monoamine oxidase A (MAOA) | M68840 | NM_000240 | 41772_at | P | P | 1.6 | 0.148 | | | |
| monoamine oxidase A | AA420624 | NM_000240 | 41771_g_at | P | P | 1.4 | 0.230 | | | |
| NECDIN related protein | U35139 | NM_002487 | 36073_at | P | P | 1.6 | 0.245 | | | |
| regulatory solute carrier protein, | X82877 | NM_006511 | 31695_g_at | A | P | 1.6 | 0.916 | | | |
| family 1, member 1 | | | | | | | | | | |
| TTK protein kinase | M86699 | NM_003318 | 572_at | P | P | 1.6 | 0.196 | | | |
| fms-related tyrosine kinase 4, | X69878 | NM_002020 | 403_s_at | A | P | 1.5 | 0.403 | + | + | 1.1 |
| VEGFR-3 | | | | | | | | | | |
| TSC403, similar to lysosome- | AB013924 | NM_014398 | 37168_at | P | P | 1.5 | 0.164 | | | |
| associated membrane glycoprotein | | | | | | | | | | |
| HMG-2 | X62534 | | 38065_at | P | P | 1.5 | 0.105 | | | 1.1 |
| Homo sapiens clone 24416 mRNA | AF052159 | | 35342_at | P | P | 1.5 | 0.253 | | | |
| sequence | | | | | | | | | | |
| calcitonin receptor-like | L76380 | NM_005795 | 34995_at | P | P | 1.5 | 0.509 | | | 1.4 |
| KIAA0582 protein | AI761647 | NM_015147 | 40191_s_at | M | P | 1.5 | 0.558 | | | 1.4 |
| cDNA DKFZp434B102 (from clone | AL080192 | | 38630_at | A | P | 1.5 | 0.719 | | | |
| DKFZp434B102) | | | | | | | | | | |
| cDNA DKFZp586G1922 (from clone | AL080110 | | 39600_at | P | P | 1.5 | 0.160 | | | |
| DKFZp586G1922) | | | | | | | | | | |
| Acyl-CoA synthetase 3 | D89053 | NM_004457 | 33880_at | P | P | 1.5 | 0.264 | | | |
| fatty-acid-Coenzyme A ligase, long- | AA977580 | NM_004457 | 33881_at | P | P | 1.0 | 0.120 | | | 1.7 |
| chain 3 | | | | | | | | | | |
| STAT induced STAT inhibitor-2 | AF037989 | | 38994_at | A | P | 1.5 | 0.391 | | | |
| Homeotic Protein Hox5.4 | HG3502-HT3696 | | 696_at | P | P | 1.5 | 0.181 | | | |
| hypothetical protein FLJ13910, | AL050139 | NM_022780 | 36580_at | P | P | 1.5 | 0.228 | | | |
| cDNA DKFZp586M141 (from clone | | | | | | | | | | |
| DKFZp586M141) | | | | | | | | | | |
| cDNA DKFZp586N012 (from clone | AL049471 | | 41690_at | P | P | 1.4 | 0.320 | | | |
| DKFZp586N012) | | | | | | | | | | |
| UbcH10, ubiquitin carrier protein E2-C | U73379 | NM_007019 | 1651_at | P | P | 1.4 | 0.022 | | | 1.1 |
| cyclin-dependent kinase inhibitor 3, | L25876 | NM_005192 | 1599_at | P | P | 1.4 | 0.431 | | | |
| protein tyrosine phosphatase (CIP2) | | | | | | | | | | |
| glycogen phosphorylase (PYGL) | AF046798 | | 37215_at | P | P | 1.4 | 0.423 | | | 1.5 |
| Angiopoietin-2 | AF004327 | NM_001147 | 1951_at | P | P | 1.4 | 0.175 | + | | 1.2 |
| Angiopoietin-2 | AF004327 | NM_001147 | 37461_at | P | P | 1.2 | 0.134 | + | | |
| forkhead box M1 | U74612 | NM_021953 | 34715_at | M | P | 1.4 | 0.367 | | | 1.4 |
| potentially prenylated protein | AF041434 | NM_007079 | 36008_at | A | P | 1.4 | 0.094 | | | 2.3 |
| tyrosine phosphatase hPRL-3 | | | | | | | | | | |
| RAB31, Low Mr GTP-binding | U59877 | NM_006868 | 33371_s_at | P | P | 1.4 | 0.299 | | | |
| protein of the Rab subfamily | | | | | | | | | | |
| RAB31, member RAS oncogene | AI189226 | NM_006868 | 33372_at | P | P | 1.2 | 0.444 | | | |
| family | | | | | | | | | | |
| myosin VIIA | U39226 | NM_000260 | 33197_at | P | P | 1.4 | 0.038 | | | 1.2 |
| Grb2-associated binder-1, docking | U43885 | NM_002039 | 1249_at | A | P | 1.4 | 0.073 | | | |
| protein related to IRS-1 | | | | | | | | | | |
| lamin B1 | L37747 | | 37985_at | P | P | 1.4 | 0.643 | | | |
| minichromosome maintenance | D84557 | NM_005915 | 40117_at | P | P | 1.4 | 0.170 | | | 1.8 |
| deficient (mis5, S. pombe) 6 | | | | | | | | | | |
| HsMcm6 | | | | | | | | | | |
| cyclin B1 | M25753 | | 1945_at | P | P | 1.4 | 0.398 | | | |
| cyclin B1 | M25753 | | 34736_at | P | P | 1.3 | 0.160 | | | |
| RTP, N-myc downstream regulated | D87953 | NM_006096 | 36933_at | P | P | 1.4 | 0.131 | | | |
| alpha2,3-sialyltransferase | AB022918 | NM_006100 | 39298_at | P | P | 1.4 | 0.150 | | | |
| ADP-ribosylation factor-like protein 4 | U73960 | NM_005738 | 33796_at | P | P | 1.4 | 0.281 | | | |
| centromere protein F (350/400 kD, | U30872 | NM_016343 | 37302_at | A | P | 1.4 | 0.245 | | | |
| mitosin) | | | | | | | | | | |
| paternally expressed 10, KIAA1051 | AB028974 | NM_015068 | 39696_at | P | P | 1.4 | 0.300 | | | 4.2 |
| tubulin, alpha 1 (testis specific) | X06956 | | 36591_at | M | P | 1.4 | 0.300 | | | 1.8 |
| KIAA0101 | D14657 | NM_014736 | 38116_at | P | P | 1.4 | 0.409 | | | |
| KIAA0128, septin 2 | D50918 | | 38826_at | P | P | 1.4 | 0.381 | | | 1.8 |
| protein phosphatase 2, regulatory | Z69030 | NM_002719 | 40785_g_at | P | P | 1.4 | 0.453 | | | |
| subunit B (B56), gamma | | | | | | | | | | |
| deoxycytidine kinase | M60527 | NM_000788 | 886_at | A | P | 1.3 | 0.455 | | | |
| integrin beta 3 binding protein | U37139 | NM_014288 | 38501_s_at | P | P | 1.3 | 0.171 | | | |
| (beta3-endonexin) | | | | | | | | | | |
| TAL1 (SCL) interrupting locus | M74558 | NM_003035 | 32767_at | M | P | 1.3 | 0.150 | | | |
| KIAA0666 | AB014566 | | 33753_at | P | P | 1.3 | 0.356 | | | |
| cAMP-specific phosphodiesterase | AF056490 | | 37676_at | P | P | 1.3 | 0.222 | | | |
| 8A, PDE8A1 | | | | | | | | | | |
| mitotic checkpoint kinase Mad3L | AF053306 | NM_001211 | 35699_at | P | P | 1.3 | 0.216 | | | 1.6 |
| (MAD3L), BUB1B | | | | | | | | | | |
| ribosomal S6 kinase | X85106 | NM_021135 | 32892_at | P | P | 1.3 | 0.145 | | | |
| HPTP epsilon (protein tyrosine | X54134 | NM_006504 | 32916_at | P | P | 1.3 | 0.100 | | | 1.0 |
| phosphatase epsilon) | | | | | | | | | | |
| Lyn tyrosine kinase, v-yes-1 | M79321 | NM_002350 | 2024_s_at | P | P | 1.3 | 0.054 | + | | |
| Yamaguchi sarcoma viral related | | | | | | | | | | |
| oncogene homolog | | | | | | | | | | |
| lyn tyrosine kinase, v-yes-1 | M16038 | NM_002350 | 1402_at | P | P | 1.3 | 0.382 | + | | |
| Yamaguchi sarcoma viral related | | | | | | | | | | |
| oncogene homolog | | | | | | | | | | |
| lyn tyrosine kinase | M16038 | NM_002350 | 32616_at | P | P | 1.2 | 0.066 | + | | |
| brachyury variant A (TBX1), T-box 1 | AF012130 | NM_005992 | 32285_g_at | P | P | 1.3 | 0.352 | | | |
| transcription factor | | | | | | | | | | |
| mki67a mRNA (long type) for | X65550 | NM_002417 | 418_at | A | P | 1.3 | 0.357 | | | 1.5 |
| antigen of monoclonal antibody Ki- | | | | | | | | | | |
| 67 | | | | | | | | | | |
| protein tyrosine phosphatase receptor | U81561 | NM_002847 | 36160_s_at | P | P | 1.3 | 0.193 | | | |
| pi (PTPRP) | | | | | | | | | | |
| cbl-b | U26710 | NM_004351 | 514_at | A | P | 1.3 | 0.482 | | | |
| Cyclin A2 | X51688 | NM_001237 | 1943_at | P | P | 1.3 | 0.277 | | | |
| nucleoside phosphorylase | X00737 | NM_000270 | 430_at | P | P | 1.3 | 0.272 | | | |
| TNF-related apoptosis inducing | U37518 | NM_003810 | 1715_at | P | P | 1.3 | 0.316 | | | |
| ligand TRAIL | | | | | | | | | | |
| phosphodiesterase 4B, cAMP- | L20971 | NM_002600 | 33705_at | P | P | 1.3 | 0.275 | | | |
| specific | | | | | | | | | | |
| nidogen (enactin) | M30269 | NM_002508 | 35366_at | P | P | 1.3 | 0.050 | | | |
| HYA22 protein | D88153 | NM_005808 | 40196_at | P | P | 1.3 | 0.150 | | | 1.3 |
| phosphatidic acid phosphatase type | AF014402 | NM_003711 | 34797_at | P | P | 1.3 | 0.191 | | | |
| 2A | | | | | | | | | | |
| KIAA0512, ALEX2 | AB011084 | NM_014782 | 36057_at | P | P | 1.2 | 0.268 | | | |
| thromboxane A2 receptor | D38081 | NM_001060 | 336_at | M | P | 1.2 | 0.385 | | | 2.4 |
| trefoil factor 3 (intestinal) | AI985964 | NM_003226 | 37897_s_at | P | P | 1.2 | 0.183 | | | |
| G-2 and S-phase expressed 1 | AL031588 | NM_016426 | 41660_at | A | P | 1.2 | 0.424 | | | 3.1 |
| ADP-ribosyltransferase (NAD+; poly | AJ236876 | NM_005484 | 34756_g_at | M | P | 1.2 | 0.362 | | | 1.1 |
| (ADP-ribose) polymerase)-like 2 | | | | | | | | | | |
| serine/threonine kinase 12 | AF015254 | NM_004217 | 33266_at | P | P | 1.2 | 0.126 | | | |
| Tubulin, Alpha 1, Isoform 44 | HG2259-HT2348 | | 330_s_at | P | P | 1.2 | 0.096 | | | 1.1 |
| lamin B receptor | L25931 | NM_002296 | 288_s_at | P | P | 1.2 | 0.141 | | | |
| KIAA0429 | AB007889 | NM_014751 | 37363_at | P | P | 1.2 | 0.150 | | | 2.5 |
| transcription factor 4 | M74719 | NM_003199 | 36605_at | P | P | 1.2 | 0.050 | | | 1.1 |
| syndecan 3 (N-syndecan), KIAA0468 | AB007937 | NM_014654 | 32092_at | P | P | 1.2 | 0.206 | | | 1.0 |
| RECK protein precursor | AA099265 | NM_021111 | 35236_g_at | P | P | 1.2 | 0.173 | | | |
| Putative prostate cancer tumor | U42349 | NM_006765 | 36852_at | P | P | 1.1 | 0.082 | | | 1.1 |
| suppressor | | | | | | | | | | |
| protein phosphatase 1, regulatory | AB020630 | | 41577_at | P | P | 1.1 | 0.082 | | | |
| (inhibitor) subunit | | | | | | | | | | |
| PDZ and LIM domain 1 (elfin) | U90878 | NM_020992 | 36937_s_at | P | P | 1.1 | 0.096 | | | |
| hypothetical protein from clone 643 | AF091087 | NM_020467 | 34176_at | P | P | 1.1 | 0.096 | | | 1.1 |
| p53-regulated DDA3 | AA926959 | | 37347_at | P | P | 1.1 | 0.058 | | | |
| KIAA0062 | D31887 | | 38797_at | P | P | 1.1 | 0.058 | | | |
| medium-chain acyl-CoA | M91432 | | 37532_at | P | P | 1.1 | 0.308 | | | |
| dehydrogenase | | | | | | | | | | |
| gap junction protein, alpha 1, 43 kD | M65188 | NM_000165 | 2018_at | P | P | 1.1 | 0.329 | | | |
| (connexin 43) | | | | | | | | | | |
| MyoD family inhibitor | U78313 | NM_005586 | 38156_at | P | P | 1.1 | 0.381 | | | |
| endo/exonuclease Mre11 (MRE11A) | AF073362 | NM_005591 | 32869_at | P | P | 1.1 | 0.642 | | | |
| nuclear receptor subfamily 2, group | X16155 | NM_005654 | 39294_at | A | P | 1.0 | 0.446 | | | |
| F, member 1 | | | | | | | | | | |

¹A measurement indicating whether the transcript was detected (present, P), not detected (absent, A) or marginally detected (marginal, M; also if P in one experiment but A in another)
²The change in expression level for a transcript between two independently harvested BECs and LECs (=total of 4 comparisons). The change is expressed as the log2 ratio.
³Standard deviation of the change in the expression level (in 4 comparisons)
NB = Northern blot,
IF = immunofluorescence

TABLE 4
Blood Vascular EC (222 genes)

| | | | | | | Gene expression | | | | |
|---|---|---|---|---|---|---|---|---|---|---|
| | | | | | | analysis | | Confirmed | | |
| | accession | | Affymetrix | Detection¹ | | signal log | | by: | | signal log |
| | numbers | | ID | BEC | LEC | ratio² | s.d.³ | NB | IF | ratio |
| p27 mRNA, interferon alpha- | X67325 | NM_005532 | 425_at | P | A | 8.3 | 0.620 | | | 1.5 |
| inducible protein 27 | | | | | | | | | | |
| ribonuclease A (RNase A), | D26129 | NM_002933 | 37402_at | P | A | 7.2 | 0.208 | | | 2.7 |
| pancreatic | | | | | | | | | | |
| hematopoietic neural membrane | U87947 | NM_001425 | 39182_at | P | A | 5.9 | 0.381 | | | 1.6 |
| protein (HNMP-1) | | | | | | | | | | |
| N-cadherin | M34064 | NM_001792 | 2054_g_at | P | A | 5.7 | 1.345 | | + | |
| N-cadherin | M34064 | NM_001792 | 2053_at | P | P | 3.7 | 0.514 | | + | |
| interleukin 8 (IL8) | M28130 | NM_000584 | 1369_s_at | P | A | 5.3 | 1.477 | + | | |
| interleukin 8, beta- | M17017 | NM_000584 | 35372_r_at | P | P | 2.8 | 0.406 | + | | 1.1 |
| thromboglobulin-like protein | | | | | | | | | | |
| precursor | | | | | | | | | | |
| tyrosine kinase receptor (axl) | M76125 | NM_001699 | 38433_at | P | A | 5.1 | 1.112 | + | | 1.0 |
| Tyrosine Kinase, Receptor Axl, Alt. | HG162- | | 1278_at | P | A | 4.7 | 0.937 | + | | 1.1 |
| Splice 2 | HT3165 | | | | | | | | | |
| cell surface glycoprotein CD44 | L05424 | | 1126_s_at | P | A | 4.9 | 1.527 | | | 1.9 |
| (CD44) | | | | | | | | | | |
| cell adhesion molecule (CD44) | M59040 | NM_000610 | 2036_s_at | P | A | 1.9 | 0.402 | | | 2.6 |
| hyaluronate receptor (CD44) | L05424 | | 40493_at | P | P | 1.9 | 0.136 | | | 2.0 |
| vascular endothelial growth factor | U43142 | NM_005429 | 159_at | P | A | 4.6 | 0.850 | + | | |
| related protein VRP, VEGF-C | | | | | | | | | | |
| Vascular endothelial growth factor C | X94216 | NM_005429 | 1934_s_at | P | A | 4.4 | 1.342 | + | | |
| collagen type XIII, alpha 1 | M33653 | NM_005203 | 38952_s_at | P | A | 4.5 | 0.213 | | | 2.7 |
| (=COL4A2) | | | | | | | | | | |
| collagen type XIII, alpha-1 | M59217 | NM_005203 | 38951_at | P | A | 3.6 | 1.683 | | | 1.1 |
| collagen alpha-2 type I | K01079 | | 32306_g_at | P | A | 4.5 | 2.161 | | | |
| collagen alpha-2 type I | K01079 | | 32305_at | P | A | 2.8 | 1.464 | | | |
| collagen, type I, alpha 2 | V00503 | NM_000089 | 32307_s_at | P | A | 2.4 | 1.394 | | | |
| proteoglycan 1 | X17042 | NM_002727 | 32227_at | P | P | 4.3 | 0.385 | | | 1.0 |
| phospholipase A2, group IVA, | M72393 | | 35938_at | P | A | 4.3 | 2.398 | | | 1.9 |
| calcium-dependent phospholipid- | | | | | | | | | | |
| binding protein (PLA2) | | | | | | | | | | |
| carbohydrate (keratan sulfate Gal-6) | AB003791 | NM_003654 | 41395_at | P | P | 4.2 | 0.232 | | | |
| sulfotransferase | | | | | | | | | | |
| tropomyosin 2 (beta), fibroblast | M12125 | NM_003289 | 32314_g_at | P | A | 4.2 | 1.845 | | | |
| tropomyosin | | | | | | | | | | |
| chondroitin sulfate proteoglycan 2 | X15998 | NM_004385 | 38111_at | P | A | 4.1 | 1.746 | | | 1.5 |
| (versican) | | | | | | | | | | |
| chondroitin sulfate proteoglycan 2 | X15998 | NM_004385 | 38112_g_at | P | A | 2.0 | 1.219 | | | |
| (versican) | | | | | | | | | | |
| latent transforming growth factor- | Z37976 | NM_000428 | 37906_at | P | P | 4.1 | 0.381 | | | 1.1 |
| beta binding protein (LTBP-2) | | | | | | | | | | |
| interleukin 6 (interferon, beta 2) | X04430 | NM_000600 | 38299_at | P | A | 4.0 | 0.776 | | | 2.0 |
| bone morphogenetic protein-4 | U43842 | NM_001202 | 40333_at | P | A | 4.0 | 0.883 | | | |
| (hBMP-4) | | | | | | | | | | |
| bone morphogenetic protein 2B, | M22490 | NM_001202 | 1114_at | P | A | 2.6 | 1.146 | | | |
| BMP-4 | | | | | | | | | | |
| sarcolectin, keratin 7 | AJ238246 | NM_005556 | 41294_at | P | P | 3.9 | 0.631 | | | |
| neuronal cell adhesion molecule, | AB002341 | NM_005010 | 37286_at | P | A | 3.9 | 1.642 | | | 1.8 |
| KIAA0343 | | | | | | | | | | |
| neuronal cell adhesion molecule, | U55258 | NM_005010 | 37288_g_at | P | A | 1.5 | 0.309 | | | |
| hBRAVO/Nr-CAM precursor | | | | | | | | | | |
| matrix metalloproteinase 1 | M13509 | NM_002421 | 38428_at | P | P | 3.9 | 0.521 | + | | |
| (interstitial collagenase), skin | | | | | | | | | | |
| collagenase | | | | | | | | | | |
| stem cell factor, KIT ligand | M59964 | NM_000899 | 597_at | P | A | 3.9 | 0.554 | + | | |
| uPA | X02419 | NM_002658 | 37310_at | P | A | 3.8 | 0.282 | | | 3.0 |
| plasminogen activator inhibitor-1 | J03764 | NM_000602 | 672_at | P | P | 3.7 | 0.161 | | | 1.6 |
| plasminogen activator inhibitor 1 | M14083 | NM_000602 | 38125_at | P | P | 2.9 | 0.118 | | | 1.9 |
| selectin P, CD62, granule | M25322 | NM_003005 | 40366_at | P | P | 3.6 | 1.869 | | | 1.2 |
| membrane protein-140 (GMP-140) | | | | | | | | | | |
| precursor | | | | | | | | | | |
| latrophilin-2 | AJ131581 | NM_012302 | 34174_s_at | P | A | 3.6 | 0.098 | | | 1.1 |
| actin, alpha 2 | X13839 | NM_001613 | 32755_at | P | P | 3.6 | 1.067 | | | |
| fibroblast activation protein, alpha | U09278 | NM_004460 | 39945_at | P | A | 3.6 | 0.789 | | | 1.2 |
| regulator of G-protein signalling 20 | AF060877 | NM_003702 | 41086_at | P | A | 3.5 | 0.615 | | | |
| IGF-II mRNA-binding protein 3 | U97188 | NM_006547 | 37558_at | P | P | 3.5 | 0.528 | | | 2.1 |
| retina cDNA randomly primed | W28438 | | 36497_at | P | A | 3.5 | 0.414 | | | |
| sublibrary, EST | | | | | | | | | | |
| brain acid-soluble protein 1, | AF039656 | NM_006317 | 32607_at | P | A | 3.4 | 0.104 | | | 1.6 |
| neuronal tissue-enriched acidic | | | | | | | | | | |
| protein (NAP-22) | | | | | | | | | | |
| profilin 2 | AL096719 | NM_002628 | 38839_at | P | P | 3.4 | 0.111 | | | 1.6 |
| profilin 2 | L10678 | NM_002628 | 38840_s_at | P | P | 3.1 | 0.076 | | | 1.6 |
| Na,K-ATPase beta-1 subunit | U16799 | NM_001677 | 37669_s_at | P | A | 3.4 | 0.249 | | | |
| Claudin-7 | AJ011497 | NM_001307 | 38482_at | P | A | 3.4 | 0.798 | | | |
| normal gingiva | U51712 | | 39698_at | P | A | 3.4 | 0.391 | | | 1.1 |
| a disintegrin and metalloproteinase | AB009672 | NM_003812 | 40350_at | P | A | 3.4 | 0.600 | | | |
| domain 23 | | | | | | | | | | |
| COL8A1 mRNA for alpha 1(VIII) | X57527 | NM_001850 | 37459_at | P | A | 3.3 | 0.819 | + | | 1.7 |
| collagen | | | | | | | | | | |
| signal transducer and activator of | AF067575 | | 41222_at | P | P | 3.3 | 0.338 | + | + | 1.8 |
| transcription 6 (STAT6) | | | | | | | | | | |
| transcription factor IL-4 Stat, | U16031 | NM_003153 | 845_at | P | A | 2.1 | 0.493 | + | + | 1.6 |
| STAT6 | | | | | | | | | | |
| lipocortin-III, annexin A3 | M20560 | NM_005139 | 31792_at | P | P | 3.3 | 0.265 | | | 1.1 |
| intercellular adhesion molecule 1 | M24283 | NM_000201 | 32640_at | P | A | 3.2 | 0.184 | + | | |
| (CD54), major group rhinovirus | | | | | | | | | | |
| receptor precusor | | | | | | | | | | |
| solute carrier family 1 | U08989 | NM_004170 | 38268_at | P | A | 3.2 | 0.687 | | | 1.3 |
| (neuronal/epithelial high affinity | | | | | | | | | | |
| glutamate transporter, system Xag) | | | | | | | | | | |
| solute carrier family 1 | AI928365 | NM_004170 | 38267_at | P | A | 2.6 | 0.524 | | | 1.2 |
| (neuronal/epithelial high affinity | | | | | | | | | | |
| glutamate transporter, system Xag) | | | | | | | | | | |
| p53 inducible protein | L47738 | | 37579_at | P | A | 3.2 | 0.249 | | | 1.0 |
| dihydropyrimidine dehydrogenase, | U20938 | NM_000110 | 38220_at | P | A | 3.2 | 0.306 | | | |
| DPYD | | | | | | | | | | |
| natural killer cell transcript 4 | AA631972 | NM_004221 | 39119_s_at | P | P | 3.1 | 0.082 | | | 1.6 |
| PFTAIRE protein kinase 1, | AB020641 | NM_012395 | 36502_at | P | A | 3.1 | 0.841 | | | |
| KIAA0834 | | | | | | | | | | |
| RGP4, regulator of G-protein | U27768 | NM_005613 | 34272_at | P | A | 3.0 | 0.396 | | | 1.5 |
| signalling 4 | | | | | | | | | | |
| regulator of G-protein signalling 4 | AI267373 | NM_005613 | 34273_at | P | A | 2.6 | 0.473 | | | 1.5 |
| Oncogene Aml1-Evi-1, Fusion | HG4058- | | 1882_g_at | P | M | 2.9 | 0.590 | | | 1.5 |
| Activated | HT4328 | | | | | | | | | |
| Oncogene Aml1-Evi-1, Fusion | HG4058- | | 1881_at | P | A | 2.0 | 0.374 | | | |
| Activated | HT4328 | | | | | | | | | |
| adenylyl cyclase-associated protein 2 | N90755 | NM_006366 | 33405_at | P | A | 2.8 | 0.596 | | | 1.5 |
| clusterin (complement lysis | M25915 | NM_001831 | 36780_at | P | P | 2.8 | 0.161 | | | |
| inhibitor, SP-40, 40, sulfated | | | | | | | | | | |
| glycoprotein 2, apolipoprotein J) | | | | | | | | | | |
| ADP ribosylation factor-like 7 | AB016811 | NM_005737 | 39829_at | P | A | 2.7 | 0.531 | | | |
| H factor (complement)-like 1 | M65292 | NM_002113 | 32249_at | P | A | 2.7 | 0.589 | | | |
| RNA helicase-related protein, | H68340 | NM_007372 | 41446_f_at | P | P | 2.7 | 0.296 | | | |
| metallothionein-If | | | | | | | | | | |
| stimulated trans-acting factor | X82200 | NM_006074 | 36825_at | P | A | 2.7 | 0.730 | | | 6.2 |
| (50 kDa) Staf50 | | | | | | | | | | |
| cyclooxygenase-2 (hCox-2) | U04636 | NM_000963 | 1069_at | P | A | 2.6 | 1.281 | | | |
| GRO1 oncogene, melanoma growth | X54489 | NM_001511 | 408_at | P | A | 2.6 | 0.369 | | | |
| stimulatory activity (MGSA) | | | | | | | | | | |
| NRGN, neurogranin | X99076 | NM_006176 | 33925_at | M | A | 2.6 | 1.237 | | | |
| homologue of mouse dkk-1 | AB020315 | | 35977_at | P | A | 2.5 | 0.398 | | | |
| gastrointestinal tumor-associated | J04152 | NM_002353 | 291_s_at | P | P | 2.5 | 0.139 | | | |
| antigen GA733-1, tumor-associated | | | | | | | | | | |
| calcium signal transducer 2 | | | | | | | | | | |
| laminin | Z15008 | NM_005562 | 35280_at | P | A | 2.5 | 0.824 | | | |
| transgelin, 22 kDa smooth muscle | M95787 | NM_003186 | 36931_at | P | A | 2.5 | 0.980 | | | |
| protein (SM22) | | | | | | | | | | |
| JE gene encoding a monocyte | M28225 | | 34375_at | P | P | 2.4 | 0.186 | | | 3.6 |
| secretory protein | | | | | | | | | | |
| zinc finger protein 238, RP58 | AJ223321 | NM_006352 | 35824_at | P | A | 2.4 | 0.498 | | | 3.2 |
| cathepsin C | X87212 | NM_001814 | 133_at | P | P | 2.4 | 0.244 | | | |
| tissue-type plasminogen activator | M15518 | NM_000930 | 33452_at | P | A | 2.4 | 0.479 | | | |
| (t-PA) | | | | | | | | | | |
| sushi-repeat protein | AF060567 | NM_014467 | 37805_at | P | A | 2.4 | 0.670 | | | |
| annexin A6 | D00510 | NM_001155 | 39082_at | P | A | 2.4 | 0.181 | | | 1.2 |
| EphrinB1 | U09303 | NM_004429 | 39721_at | P | A | 2.4 | 0.946 | | | |
| EphrinB1 | U09303 | NM_004429 | 188_at | P | A | 1.2 | 0.489 | | | |
| TFEC isoform (transcription factor | D43945 | NM_012252 | 34470_at | P | A | 2.4 | 0.028 | | | 3.1 |
| EC) | | | | | | | | | | |
| small inducible cytokine A2, | M26683 | NM_002982 | 875_g_at | P | P | 2.3 | 0.283 | | | 3.5 |
| (monocyte chemotactic protein1) | | | | | | | | | | |
| small inducible cytokine A2 | M26683 | NM_002982 | 874_at | P | A | 1.2 | 0.385 | | | |
| (monocyte chemotactic protein 1) | | | | | | | | | | |
| endothelial cell protein C/APC | L35545 | NM_006404 | 647_at | P | P | 2.3 | 0.259 | | | 2.2 |
| receptor (EPCR) | | | | | | | | | | |
| transglutaminase 2 (TGase) | M55153 | NM_004613 | 231_at | P | M | 2.3 | 0.413 | | | |
| transglutaminase (TGase) | M55153 | NM_004613 | 38404_at | P | P | 1.6 | 0.093 | | | |
| human metallothionein-If | M10943 | | 31622_f_at | P | M | 2.3 | 0.230 | | | |
| transforming growth factor beta- | M77349 | NM_000358 | 1385_at | P | A | 2.3 | 0.951 | | | |
| induced (BIGH3) | | | | | | | | | | |
| ENO2 gene for neuron specific | X51956 | | 40193_at | P | A | 2.3 | 0.121 | | | 1.6 |
| (gamma) enolase | | | | | | | | | | |
| FAT tumor suppressor (Drosophila) | X87241 | NM_005245 | 40454_at | P | A | 2.3 | 1.204 | | | |
| homolog | | | | | | | | | | |
| malignant cell expression-enhanced | S82470 | NM_024298 | 181_g_at | P | P | 2.2 | 0.469 | | | |
| gene/tumor progression-enhanced | | | | | | | | | | |
| gene | | | | | | | | | | |
| malignant cell expression-enhanced | S82470 | NM_024298 | 180_at | P | A | 1.7 | 0.420 | | | |
| gene/tumor progression-enhanced | | | | | | | | | | |
| gene | | | | | | | | | | |
| cDNA DKFZp566G0746 (from | AL050078 | | 39324_at | P | A | 2.2 | 1.281 | | | |
| clone DKFZp566G0746) | | | | | | | | | | |
| lysyl oxidase-like 2 | U89942 | NM_002318 | 33127_at | P | P | 2.2 | 0.274 | | | |
| ras-related C3 botulinum toxin | M64595 | NM_002872 | 32737_at | P | P | 2.2 | 0.143 | | | 3.5 |
| substrate 2 (rho family, small GTP | | | | | | | | | | |
| binding protein Rac2) | | | | | | | | | | |
| endothelial leukocyte adhesion | M24736 | NM_000450 | 265_s_at | P | A | 2.2 | 0.448 | | | |
| molecule 1 (ELAM-1), selectin E | | | | | | | | | | |
| laminin, alpha 5, KIAA0533 | AB011105 | | 41610_at | P | P | 2.2 | 0.146 | | | |
| placenta growth factor (P1GF) | X54936 | NM_002632 | 793_at | P | P | 2.2 | 0.301 | + | | 1.0 |
| ALL1-fused gene from | U16954 | NM_006818 | 36941_at | P | P | 2.2 | 0.592 | | | |
| chromosome 1q, AF1q | | | | | | | | | | |
| stromelysin-2, MMP-10 | X07820 | NM_002425 | 1006_at | P | P | 2.1 | 0.159 | | | 1.0 |
| metallothionein-I-A | K01383 | | 31623_f_at | P | P | 2.1 | 0.238 | | | |
| collagen VI alpha-1 | X15880 | | 38722_at | P | A | 2.1 | 0.898 | | | |
| mad protein homolog (hMAD-3) | U68019 | NM_005902 | 1433_g_at | P | A | 2.1 | 0.269 | | | 1.1 |
| mad protein homolog (hMAD-3) | U68019 | NM_005902 | 38944_at | P | A | 1.8 | 0.367 | | | 1.4 |
| mad protein homolog (hMAD-3) | U68019 | NM_005902 | 1454_at | P | A | 2.0 | 0.304 | | | |
| integral membrane protein 2A | AL021786 | | 40775_at | P | P | 2.1 | 0.570 | | | |
| interleukin 1 receptor-like 1 | D12763 | NM_003856 | 40322_at | P | A | 2.1 | 0.718 | | | |
| high-mobility group (nonhistone | X92518 | | 35200_at | P | A | 2.0 | 0.177 | | | |
| chromosomal) protein isoform I-C | | | | | | | | | | |
| (HMGI-C) | | | | | | | | | | |
| epidermal growth factor receptor | U12535 | NM_004447 | 1467_at | P | A | 2.0 | 0.710 | | | 1.0 |
| kinase substrate (Eps8) | | | | | | | | | | |
| lactate dehydrogenase B | X13794 | NM_002300 | 33819_at | P | P | 2.0 | 0.029 | | | 1.6 |
| mRNA for unknown product | D29810 | | 40227_at | P | A | 2.0 | 1.170 | | | |
| hypothetical protein | AL033377 | | 36014_at | P | A | 2.0 | 0.137 | | | 1.1 |
| DKFZp564D0462 | | | | | | | | | | |
| lysyl hydroxylase isoform 2 | U84573 | NM_000935 | 34795_at | P | P | 2.0 | 0.157 | | | |
| (PLOD2) | | | | | | | | | | |
| follistatin-like 3, follistatin-related | U76702 | NM_005860 | 33900_at | P | A | 2.0 | 0.075 | | | 2.6 |
| protein (FLRG) | | | | | | | | | | |
| Homo sapiens clone 24674 mRNA | AF070578 | | 36758_at | P | A | 2.0 | 0.612 | | | 1.8 |
| sequence | | | | | | | | | | |
| L-iditol-2 dehydrogenase | L29254 | | 38763_at | P | A | 1.9 | 0.140 | | | |
| neuronal pentraxin 1 | U61849 | NM_002522 | 37921_at | P | A | 1.9 | 0.744 | | | 2.5 |
| hypothetical protein from clones | U90908 | NM_021226 | 34010_at | P | A | 1.9 | 0.870 | | | |
| 23549 and 23762 | | | | | | | | | | |
| UDP-N-acetylglucosamine | AB011004 | NM_003115 | 41242_at | P | P | 1.9 | 0.342 | | | |
| pyrophosphorylase | | | | | | | | | | |
| zinc finger protein 185 (LIM | Y09538 | NM_007150 | 32139_at | P | A | 1.8 | 0.062 | | | 1.6 |
| domain) | | | | | | | | | | |
| four and a half LIM domains 2, | U29332 | NM_001450 | 38422_s_at | P | P | 1.8 | 0.229 | | | |
| heart protein (FHL-2) | | | | | | | | | | |
| mitogen-activated protein kinase- | U09578 | NM_004635 | 1637_at | P | A | 1.8 | 0.454 | | | 1.4 |
| activated protein kinase 3, | | | | | | | | | | |
| MAPKAP kinase (3pK) | | | | | | | | | | |
| metallothionein 1E (functional) | R92331 | | 36130_f_at | P | P | 1.8 | 0.131 | | | |
| TU3A protein | AF035283 | NM_007177 | 38044_at | P | A | 1.8 | 0.298 | | | 1.8 |
| metallothionein 1H | R93527 | NM_005951 | 39594_f_at | P | P | 1.8 | 0.415 | | | |
| guanylate binding protein isoform II | M55543 | NM_004120 | 32700_at | P | P | 1.8 | 0.304 | | | 1.1 |
| (GBP-2) | | | | | | | | | | |
| soluble vascular endothelial cell | U01134 | NM_002019 | 1964_g_at | P | M | 1.8 | 0.384 | + | | |
| growth factor receptor 1 (sVEGFR- | | | | | | | | | | |
| 1) | | | | | | | | | | |
| R-Ras | M14949 | | 38338_at | P | P | 1.8 | 0.119 | | | |
| R-ras | M14949 | | 1879_at | P | P | 1.2 | 0.330 | | | 1.5 |
| creatine transporter (SLC6A8), | U36341 | NM_005629 | 40926_at | P | A | 1.8 | 0.305 | | | |
| solute carrier family 6, member 8 | | | | | | | | | | |
| target of myb1 (chicken) homolog, | Z82244 | NM_005488 | 33802_at | P | P | 1.8 | 0.149 | | | |
| Heme Oxygenase 1 (HO-1) | | | | | | | | | | |
| procollagen-lysine, 2-oxoglutarate | L06419 | NM_000302 | 36184_at | P | P | 1.8 | 0.310 | | | |
| 5-dioxygenase, lysyl hydroxylase | | | | | | | | | | |
| (PLOD) | | | | | | | | | | |
| KIAA0836 | AB020643 | | 33296_at | P | P | 1.8 | 0.203 | | | 1.8 |
| cDNA DKFZp434C171 (from clone | AL080169 | | 34183_at | P | A | 1.8 | 0.606 | | | 1.2 |
| DKFZp434C171) | | | | | | | | | | |
| IL-4-R mRNA for the interleukin 4 | X52425 | NM_000418 | 404_at | P | P | 1.7 | 0.244 | | | |
| receptor | | | | | | | | | | |
| chemokine (C—C motif) receptor- | AF014958 | NM_003965 | 1445_at | P | A | 1.7 | 0.386 | | | 1.8 |
| like 2 (CCRL2), chemokine | | | | | | | | | | |
| receptor X (CKRX) | | | | | | | | | | |
| phospholipase C, beta 3 | Z16411 | NM_000932 | 364_s_at | P | A | 1.7 | 0.098 | | | |
| (phosphatidylinositol-specific) | | | | | | | | | | |
| LIM domain protein | X93510 | NM_003687 | 32610_at | P | P | 1.7 | 0.067 | | | 1.9 |
| protein kinase (cAMP-dependent, | M34181 | NM_002731 | 36215_at | P | A | 1.7 | 0.315 | | | 1.0 |
| catalytic) inhibitor beta | | | | | | | | | | |
| rho GDP-dissociation Inhibitor 2 | X69549 | NM_001175 | 1984_s_at | P | P | 1.7 | 0.163 | | | 2.4 |
| KIAA0975, imidazoline receptor | AB023192 | NM_007184 | 33916_at | P | A | 1.7 | 0.343 | | | |
| candidate | | | | | | | | | | |
| poliovirus receptor | X64116 | NM_006505 | 32698_at | P | P | 1.7 | 0.173 | | | |
| poliovirus receptor | X64116 | NM_006505 | 32699_s_at | P | P | 1.3 | 0.102 | | | |
| immediate early response 3 | S81914 | NM_003897 | 1237_at | P | P | 1.7 | 0.171 | | | |
| metallothionein 2A | AI547258 | NM_005953 | 39081_at | P | A | 1.7 | 0.247 | | | |
| tropomyosin 1 (alpha) | M19267 | NM_000366 | 36791_g_at | P | P | 1.6 | 0.184 | | | 1.0 |
| tropomyosin 1 (alpha) | Z24727 | NM_000366 | 36792_at | P | P | 1.4 | 0.094 | | | 1.3 |
| tropomyosin 1 (alpha) | M19267 | NM_000366 | 36790_at | P | P | 1.2 | 0.213 | | | |
| TRAM-like protein | D31762 | NM_012288 | 40051_at | P | P | 1.6 | 0.244 | | | 1.1 |
| E3 ubiquitin ligase SMURF2 | AA630312 | NM_022739 | 33354_at | P | P | 1.6 | 0.244 | | | 2.2 |
| EGF-containing fibulin-like | U03877 | NM_004105 | 32551_at | P | P | 1.6 | 0.152 | | | |
| extracellular matrix protein 1 | | | | | | | | | | |
| G protein-coupled receptor 56 | AJ011001 | NM_005682 | 35769_at | P | P | 1.6 | 0.075 | | | |
| c-jun proto oncogene (JUN) | J04111 | NM_002228 | 32583_at | P | A | 1.6 | 0.377 | | | |
| regulator of G-protein signalling 10, | AF045229 | NM_002925 | 33121_g_at | P | A | 1.6 | 0.064 | | | |
| RGS10 | | | | | | | | | | |
| amyloid beta (A4) precursor | U62325 | | 40148_at | P | P | 1.6 | 0.312 | | | 1.3 |
| protein-binding, family B, member | | | | | | | | | | |
| 2 (Fe65-like) | | | | | | | | | | |
| ras-related rho protein | M12174 | NM_004040 | 1826_at | P | P | 1.6 | 0.281 | | | 1.4 |
| proteasome (prosome, macropain) | AL031177 | NM_002814 | 37957_at | P | A | 1.5 | 0.265 | | | |
| 26S subunit, non-ATPase, 10 | | | | | | | | | | |
| KIAA0537 | AB011109 | NM_014840 | 33787_at | P | P | 1.5 | 0.135 | | | 1.8 |
| lysosome-associated membrane | X77196 | NM_002294 | 38403_at | P | P | 1.5 | 0.432 | | | |
| protein-2 | | | | | | | | | | |
| phospholipid transfer protein | L26232 | NM_006227 | 40081_at | P | P | 1.5 | 0.046 | | | |
| N-myristoyltransferase 2 | AF043325 | NM_004808 | 41656_at | P | P | 1.5 | 0.038 | | | |
| phosphofructokinase (PFKM) | U24183 | NM_000289 | 36196_at | P | P | 1.5 | 0.374 | | | 2.0 |
| integrin, beta 4 | X53587 | NM_000213 | 406_at | P | A | 1.5 | 0.195 | | | |
| leupaxin | AF062075 | NM_004811 | 36062_at | P | A | 1.5 | 0.231 | | | 1.3 |
| endothelin-converting-enzyme 1 | Z35307 | NM_001397 | 41726_at | P | P | 1.5 | 0.180 | | | |
| wild-type p53 activated fragment-1 | U03106 | NM_000389 | 2031_s_at | P | P | 1.5 | 0.399 | | | |
| (WAF1), cyclin-dependent kinase | | | | | | | | | | |
| inhibitor 1A (p21, Cip1) | | | | | | | | | | |
| ICAM-2, cell adhesion ligand for | X15606 | NM_000873 | 38453_at | P | P | 1.5 | 0.024 | | | 1.5 |
| LFA-1 | | | | | | | | | | |
| ICAM-2, cell adhesion ligand for | X15606 | NM_000873 | 38454_g_at | P | P | 1.5 | 0.153 | | | 2.0 |
| LFA-1 | | | | | | | | | | |
| intercellular adhesion molecule 2 | M32334 | | 590_at | P | P | 1.4 | 0.152 | | | 1.5 |
| (ICAM-2) | | | | | | | | | | |
| eukaryotic translation initiation | AF035280 | NM_014239 | 40515_at | P | P | 1.5 | 0.108 | | | |
| factor 2B, eIF-2B beta subunit | | | | | | | | | | |
| uridine phosphorylase | X90858 | NM_003364 | 37351_at | P | M | 1.5 | 0.064 | | | |
| integrin, beta 5 | X53002 | NM_002213 | 39754_at | P | P | 1.5 | 0.068 | | | |
| N-sulfoglucosamine sulfohydrolase | U30894 | NM_000199 | 35626_at | P | P | 1.5 | 0.077 | | | |
| (sulfamidase) | | | | | | | | | | |
| synaptojanin 2 | AF039945 | | 36532_at | P | A | 1.5 | 0.164 | | | 1.1 |
| metallothionein 1L | AA224832 | NM_002450 | 39120_at | P | A | 1.4 | 0.664 | | | |
| macrophage capping protein, | M94345 | NM_001747 | 38391_at | P | P | 1.4 | 0.281 | | | |
| gelsolin-like | | | | | | | | | | |
| HSPC022 protein | W68830 | NM_014029 | 32736_at | P | P | 1.4 | 0.062 | | | 2.7 |
| Human clone 137308 mRNA, | AW006742 | | 38207_at | P | A | 1.4 | 0.442 | | | |
| partial cds | | | | | | | | | | |
| protocadherin 42, PC42, | L11370 | NM_002587 | 37562_at | P | A | 1.4 | 0.166 | | | |
| protocadherin 1 (cadherin-like 1) | | | | | | | | | | |
| caspase-like apoptosis regulatory | AF005775 | NM_003879 | 1867_at | P | P | 1.4 | 0.363 | | | 1.5 |
| protein 2 (CLARP2) | | | | | | | | | | |
| caspase-like apoptosis regulatory | AF005775 | NM_003879 | 1868_g_at | P | P | 1.2 | 0.325 | | | 2.2 |
| protein 2 (CLARP2) | | | | | | | | | | |
| major vault protein, lrp | X79882 | NM_005115 | 38064_at | P | P | 1.4 | 0.252 | | | 1.0 |
| Fanconi anemia, complementation | AC004472 | NM_004629 | 33842_at | P | A | 1.4 | 0.233 | | | |
| group G | | | | | | | | | | |
| prion protein (PrP) | U29185 | NM_000311 | 36159_s_at | P | P | 1.4 | 0.342 | | | 1.1 |
| interferon-stimulated protein, 15 kDa | AA203213 | NM_005101 | 38432_at | P | A | 1.4 | 0.244 | | | |
| serine (or cysteine) proteinase | L40377 | NM_002640 | 36312_at | P | A | 1.3 | 0.360 | | | 2.2 |
| inhibitor, clade B (ovalbumin), | | | | | | | | | | |
| cytoplasmic antiproteinase 2 | | | | | | | | | | |
| (CAP2) | | | | | | | | | | |
| biglycan | J04599 | NM_001711 | 38126_at | P | P | 1.3 | 0.101 | | | |
| chemokine (C—X—C motif), | L06797 | NM_003467 | 649_s_at | P | P | 1.3 | 0.177 | + | | |
| receptor 4 (fusin) | | | | | | | | | | |
| ubiquitin carboxyl-terminal esterase | X04741 | NM_004181 | 36990_at | P | P | 1.3 | 0.117 | | | |
| L1 (ubiquitin thiolesterase) | | | | | | | | | | |
| KIAA0469 | AB007938 | NM_014851 | 37230_at | P | P | 1.3 | 0.124 | | | |
| TNF (ligand) superfamily, member | AL022310 | NM_003326 | 32319_at | P | A | 1.3 | 0.349 | | | |
| 4 (tax-transcriptionally activated | | | | | | | | | | |
| glycoprotein 1, 34 kD) | | | | | | | | | | |
| KIAA1053 | AB028976 | | 40855_at | P | P | 1.3 | 0.242 | | | 1.6 |
| NAD(P)H-quinone oxireductase | M81600 | | 38066_at | P | P | 1.3 | 0.058 | | | 1.8 |
| sushi-repeat-containing protein | U61374 | NM_006307 | 31855_at | P | P | 1.3 | 0.610 | | | 1.2 |
| integrin, alpha 5 | X06256 | NM_002205 | 39753_at | P | P | 1.3 | 0.179 | | + | 1.2 |
| enigma (LIM domain protein) | L35240 | NM_005451 | 39530_at | P | P | 1.3 | 0.396 | | | |
| ectonucleoside triphosphate | AJ133133 | NM_001776 | 32826_at | P | A | 1.3 | 0.412 | | | 1.5 |
| diphosphohydrolase 1 | | | | | | | | | | |
| transforming growth factor-beta | M60315 | NM_001718 | 39279_at | P | P | 1.3 | 0.206 | | | |
| (tgf-beta), bone morphogenetic | | | | | | | | | | |
| protein 6 | | | | | | | | | | |
| transforming growth factor-beta | M60315 | NM_001718 | 1733_at | P | P | 1.0 | 0.308 | | | |
| (tgf-beta), bone morphogenetic | | | | | | | | | | |
| protein 6 | | | | | | | | | | |
| nicotinamide N-methyltransferase, | U08021 | NM_006169 | 37032_at | P | P | 1.2 | 0.083 | | | 2.1 |
| NNMT | | | | | | | | | | |
| cDNA DKFZp564J0323 (from | AL049957 | | 39170_at | P | P | 1.2 | 0.264 | | | 1.2 |
| clone DKFZp564J0323) | | | | | | | | | | |
| thioredoxin reductase beta | AB019694 | NM_006440 | 41711_at | P | A | 1.2 | 0.206 | | | |
| f-box and leucine-rich repeat | AL049953 | | 36525_at | P | A | 1.2 | 0.300 | | | 1.2 |
| protein 2 | | | | | | | | | | |
| transcobalamin II (TCN2) | L02648 | NM_000355 | 37922_at | P | A | 1.2 | 0.342 | | | 1.2 |
| aldehyde dehydrogenase 2, | X05409 | NM_000690 | 32747_at | P | P | 1.2 | 0.117 | | | |
| mitochondrial | | | | | | | | | | |
| GTP-binding protein ragB | X90530 | NM_006064 | 39989_at | P | M | 1.2 | 0.602 | | | |
| lymphocyte antigen 75 | AF011333 | NM_002349 | 38160_at | P | A | 1.2 | 0.132 | | | 1.7 |
| GM2 activator protein | X62078 | | 35820_at | P | P | 1.2 | 0.101 | | | 1.5 |
| type 3 inositol 1,4,5-trisphosphate | U01062 | NM_002224 | 182_at | P | P | 1.2 | 0.052 | | | 1.5 |
| receptor (ITPR3) | | | | | | | | | | |
| KIAA0284 | AI828210 | | 38592_s_at | P | P | 1.2 | 0.078 | | | 1.9 |
| metallothionein I-B | M13485 | | 609_f_at | P | P | 1.2 | 0.266 | | | |
| BTG2 | U72649 | NM_006763 | 36634_at | P | P | 1.2 | 0.210 | | | |
| adenylate kinase 1 | J04809 | NM_000476 | 36997_at | P | A | 1.2 | 0.246 | | | |
| tumor necrosis factor receptor | Y09392 | NM_003790 | 41189_at | P | P | 1.2 | 0.350 | | | |
| superfamily, member 12, WSL-LR, | | | | | | | | | | |
| WSL-S1 and WSL-S2 proteins | | | | | | | | | | |
| aminopeptidase N/CD13 | M22324 | NM_001150 | 39385_at | P | P | 1.2 | 0.398 | | | |
| growth arrest and DNA-damage- | M60974 | NM_001924 | 1911_s_at | P | P | 1.2 | 0.177 | | | |
| inducible protein (gadd45) | | | | | | | | | | |
| KIAA0638 protein | AB014538 | | 37375_at | P | P | 1.2 | 0.680 | | | |
| vinculin | M33308 | NM_003373 | 36601_at | P | P | 1.2 | 0.078 | | + | |
| procollagen-proline, 2-oxoglutarate | U90441 | NM_004199 | 34390_at | P | P | 1.1 | 0.347 | | | |
| 4-dioxygenase (proline 4- | U65093 | NM_006079 | 33113_at | P | P | 1.1 | 0.164 | | | 1.2 |
| hydroxylase), alpha polypeptide II | | | | | | | | | | |
| msg1-related gene 1 (mrg1), | | | | | | | | | | |
| Cbp/p300-interacting transactivator | | | | | | | | | | |
| microsomal glutathione S- | AF026977 | NM_004528 | 39018_at | P | P | 1.1 | 0.191 | | | 1.1 |
| transferase 3 | | | | | | | | | | |
| vitamin A responsive; cytoskeleton | AF070523 | NM_006407 | 39091_at | P | P | 1.1 | 0.216 | | | 2.9 |
| related | | | | | | | | | | |
| 17-kDa protein, interferon- | M13755 | NM_005101 | 1107_s_at | P | P | 1.1 | 0.119 | | | |
| stimulated protein, 15 kDa | | | | | | | | | | |
| matrix metalloproteinase 14 | X83535 | NM_004995 | 34747_at | P | A | 1.1 | 0.487 | | | 2.4 |
| (membrane-inserted) | | | | | | | | | | |
| 4F2 cell-surface antigen, solute | J02939 | NM_002394 | 38029_at | P | P | 1.1 | 0.143 | | | |
| carrier family 3, member 2 | | | | | | | | | | |
| metallothionein-III | M93311 | NM_005954 | 870_f_at | P | P | 1.1 | 0.334 | | | |
| protein kinase (cAMP-dependent, | S76965 | NM_006823 | 36202_at | P | P | 1.1 | 0.046 | | | |
| catalytic) inhibitor alpha | | | | | | | | | | |
| protein kinase (cAMP-dependent, | S76965 | NM_006823 | 546_at | P | P | 1.0 | 0.367 | | | |
| catalytic) inhibitor alpha | | | | | | | | | | |
| reticulocalbin 1, EF-hand calcium | D42073 | NM_002901 | 40556_at | P | P | 1.1 | 0.035 | | | 1.1 |
| binding domain | | | | | | | | | | |
| lipin 1, KIAA0188 | D80010 | | 38098_at | P | P | 1.1 | 0.080 | | | 1.4 |
| protease, serine, 23 | AF015287 | NM_007173 | 40078_at | P | P | 1.0 | 0.099 | | | 1.3 |
| hect domain and RLD 2 | AF041080 | NM_004667 | 40877_s_at | P | P | 1.0 | 0.104 | | | |
| GATA-binding protein (GATA2) | M68891 | NM_002050 | 37194_at | P | P | 1.0 | 0.325 | | | 1.0 |
| agrin precursor | AF016903 | | 33454_at | P | P | 1.0 | 0.272 | | | |
| equilibrative nucleoside transporter | U81375 | NM_004955 | 33901_at | P | P | 1.0 | 0.352 | | | |
| 1 (hENT1) | | | | | | | | | | |
| coronin, actin-binding protein 2B, | AB023142 | | 34772_at | P | A | 1.0 | 0.459 | | | |
| KIAA0925 | | | | | | | | | | |
| f-box and WD-40 domain protein 3 | U07000 | NM_012165 | 537_f_at | P | M | 1.0 | 0.212 | | | |
| nonsyndromic hearing impairment | AF073308 | NM_004403 | 41872_at | P | P | 1.0 | 0.535 | | | 1.0 |
| protein (DFNA5) | | | | | | | | | | |
| actin filament associated protein | D25248 | NM_021638 | 37578_at | P | P | 1.0 | 0.218 | | | |
| TNFR-related death receptor-6 | AF068868 | NM_014452 | 35402_at | P | A | 1.0 | 0.235 | | | 1.7 |
| (DR6) | | | | | | | | | | |
| serum/glucocorticoid regulated | Y10032 | NM_005627 | 973_at | P | A | 1.0 | 0.174 | | | |
| kinase | | | | | | | | | | |
| DNase X | X90392 | NM_006730 | 37214_g_at | P | P | 1.0 | 0.507 | | | |
| DNase X | X90392 | NM_006730 | 37213_at | P | P | 1.0 | 0.376 | | | |
| fatty acid desaturase 3 | AC004770 | NM_021727 | 34224_at | P | P | 1.0 | 0.294 | | | |
| LYL-1 | M22637 | | 39971_at | P | P | 1.0 | 0.313 | | | 1.4 |
| ATP-binding cassette, sub-family C | X78338 | NM_004996 | 34016_s_at | P | A | 1.0 | 0.258 | | | 2.3 |
| (CFTR/MRP), member 1 | | | | | | | | | | |
| transmembrane protein (CD59) | M84349 | | 39351_at | P | P | 1.0 | 0.141 | | | 1.1 |
| fms-related tyrosine kinase 1, | S77812 | NM_002019 | 1545_g_at | P | P | 1.0 | 0.535 | + | | 1.9 |
| VEGFR-1 | | | | | | | | | | |

¹A measurement indicating whether the transcript was detected (present, P), not detected (absent, A) or marginally detected (marginal, M; also if P in one experiment but A in another)
²The change in expression level for a transcript between two independently harvested BECs and LECs (=total of 4 comparisons). The change is expressed as the log2 ratio.
³Standard deviation of the change in the expression level (in 4 comparisons)
NB = Northern blot,
IF = immunofluorescence

TABLE 10
Known LEC-specific genes

| | | Accession numbers | |
|---|---|---|---|
| Gene | Detection* | starting EST | possible gene |
| CD36 = | Af (S/4, 3) | R20784 | M98399 |
| COL1/TSP receptor, fatty-acid transport | | H54254 | |
| protein | | | |
| beta1-syntrophin | Af (S/4, 5) | AA447177 | L31529 |
| collectin sub-family member 12 | Af (S/4, 5) | R74387 | NM_030781 |
| a disintegrin and metalloprotease | Af (S/4, 3) | AA147933 | NM_003474 |
| domain 12 | | | |
| cytotoxic T-lymphocyte- | Af (S/4, 0) | AI733018 | NM_005214 |
| associated protein 4 | | | |
| niban protein NM_022083 niban | Af (S/3, 7) | AA554814 | NM_052966 |
| protein | | | |
| multi-PDZ-domain-containing | Af (S/3, 5) | AI738919 | NM_032622 |
| protein, LNX | | | |
| MAGE-E1 protein | Af (S/3, 2) | AI435112 | NM_030801 |
| upstream stimulatory factor 1, | Af (S/2, 6) | AA701033 | AB017568 |
| USF1 (genomic match) | | | |
| hairy/enhancer-of-split related | Af (NS/2, 6) | R61374 | NM_012258 |
| with YRPW motif 1 | | | |
| alpha-2,8-polysialyltransferase | Af (S/2, 5) | AI422986 | L41680 |
| semaphorin 6A1 | Af (S/2, 4) | W21965 | NM_020796 |
| guanine nucleotide binding | Af (S/2, 3) | AA738022 | |
| protein (G prot), gamma 2 | | | |
| integral membrane protein 3 | Af (S/2, 3) | AA128019 | NM_030926 |
| similar to mouse glucocorticoid- | Af (S/2, 0) | AI678080 | XM_070471 |
| induced gene 1 | | | |
| YAP65 (Yes-associated protein of | Af (NS/2, 0) | AL048399 | X80507 |
| 65 kDa MW) | | | |
| 17 kDa fetal brain protein | Af (NS/1, 9) | H92988 | NM_022343 |
| Kruppel-like factor 5 | Af (S/1, 8) | AI815057 | NM_001730 |
| calcitonin receptor-like, CGRP | Af (S/1, 7) | AI741128, | NM_005795, L76380 |
| type 1 receptor | | T94540 | |
| fibroblast growth factor 13, | Af (NS/1, 7) | AW014749 | NM_004114 |
| isoform 1A | | | |
| tetraspan NET-6 protein | Af (NS/1, 6) | W22687 | NM_014399 |
| ring finger protein 11 | Af (S/1, 6) | AL079648 | BC020964 |

*Af = Affymetrix, S = specific for LEC, NS = nonspecific (also expressed in BEC), numbers represent log2 ratio of the signal intensities between BEC and LEC

TABLE 11
Differentially expressed genes identified by accession number

| Gene | Detection* | starting EST | SEQ ID NO: |
|---|---|---|---|
| EST | Af (S/4, 9) | AL079386 | 1 |
| EST | Af (S/3, 7) | N21555 | 2 |
| EST | Af (S/3, 2) | AL119027 | 3 |
| EST | Af (S/2, 9) | H05299 | 4 |
| EST | Af (S/2, 8) | AA973128 | 5 |
| EST | Af (NS/2, 6) | AI128820 | |
| EST | Af (S/2, 3) | AW044647 | 6 |
| EST | Af (S/2, 2) | AI333058 | 7 |
| EST | Af (S/2, 1) | AI536067 | 8 |
| EST | Af (NS/2, 0) | AA156409 | |
| EST | Af (S/1, 9) | AI770080 | 9 |
| EST | Af (NS/1, 9) | AA456099 | |
| EST | Af (S/1, 8) | AI692645 | 10 |
| EST | Af (S/1, 7) | AL119265 | 11 |
| EST | Af (S/1, 6) | AI478114 | 12 |
| EST | Af (S/1, 6) | AI817448 | 13 |

*Af = Affymetrix, S = specific for LEC, NS = nonspecific (also expressed in BEC), numbers represent log2 ratio of the signal intensities between BEC and LEC

TABLE 12
Other Proteins Identified

| | | Accession numbers | |
|---|---|---|---|
| Gene | Detection* | starting EST | possible gene |
| KIAA1392, hypothetical protein | Af (S/5, 3) | N50545 | XM_048721 (20) |
| DKFZp762K222 | | | |
| similar to phosphoglucomutase 5 | Af (S/4, 5) | AL046941 | XM_047649 (21) |
| Similar to transmembrane | | | |
| receptor Unc5H1 | Af (S/4, 5) | R56359 | XM_030300 (22) |
| hypothetical protein MGC21854 | Af (S/3, 7) | AI659418 | NM_052862 (23) |
| KIAA1877 | Af (S/3, 4) | AW004016 | |
| similar to unnamed protein | | | |
| product | Af (NS/3, 1) | AA036952 | XM_085235 |
| unknown protein | Af (S/2, 9) | AA846091 | XM_038314 (24) |
| KIAA1058 (+missing N-term | Af (S/2, 6) | AA007697 | AB028981 (25) |
| from ests) | | | |
| similar to KIAA1673 | Af (S/2, 3) | AI948598 | XM_059607 (26) |
| similar to lysosomal amino acid | Af (S/2, 3) | AI692279 | XM_058449 (27) |
| transporter 1 | | | |
| Homo sapiens similar to | | | |
| KIAA1673 protein | Af (S/2, 3) | AI948598 | XM_059607 |
| KIAA0493 | Af (S/2, 3) | AA532655 | AB007962 (28) |
| hypothetical protein MGC2780 | Af (S/2, 3) | AI734962 | NM_025266 (29) |
| transmembrane protein 2 | Af (NS/2, 3) | NM_013390 | 57094_at |
| Novel human gene mapping to | Af (S/2, 2) | AA651889 | HS455J72 (30) |
| chomosome 1 | | | |

*Af = Affymetrix, S = specific for LEC, NS = nonspecific (also expressed in BEC), numbers represent log2 ratio of the signal intensities between BEC and LEC

In Tables 5, 6 and 12, the numbers in parentheses refer to the SEQ ID NO: in the Sequence Listing. Table 13 below correlates these sequences with polypeptide sequences SEQ ID NO:31-44 and 46 (Open reading frames, ORF's).

TABLE 13
Polypeptides corresponding to LEC-specific polynucleotides

| Accession number | Polynucleotide | Polypeptide |
|---|---|---|
| NM_021647 | SEQ ID NO: 14 | SEQ ID NO: 31 |
| NM_014817 | SEQ ID NO: 15 | SEQ ID NO: 32 |
| XM_059074 | SEQ ID NO: 16 | SEQ ID NO: 33 |
| NM_016647 | SEQ ID NO: 17 | SEQ ID NO: 34 |
| XM_048721 | SEQ ID NO: 20 | SEQ ID NO: 35 |
| XM_047649 | SEQ ID NO: 21 | SEQ ID NO: 36 |
| XM_030300 | SEQ ID NO: 22 | SEQ ID NO: 37 |
| NM_052862 | SEQ ID NO: 23 | SEQ ID NO: 38 |
| XM_039314 | SEQ ID NO: 24 | SEQ ID NO: 39 |
| AB028981 | SEQ ID NO: 25 | SEQ ID NO: 40 |
| XM_059607 | SEQ ID NO: 26 | SEQ ID NO: 41 |
| XM_058449 | SEQ ID NO: 27 | SEQ ID NO: 42 |
| NM_025266 | SEQ ID NO: 29 | SEQ ID NO: 43 |
| AL137762/HS455J72 | SEQ ID NO: 30 | SEQ ID NO: 44 |
| XM_084655 | SEQ ID NO: 45 | SEQ ID NO: 46 |

TABLE 14
Sequence identifiers for sequences in Table 3

| | accession | | | |
|---|---|---|---|---|
| | numbers | | aa SEQ ID NO: | nt SEQ ID NO: |
| lung type-I cell membrane-associated protein, | AF030428 | NM_006474 | SEQ ID NO: 65 | SEQ ID NO: 66 |
| podoplanin | | | | |
| lung type-I cell membrane-associated protein, | AI660929 | NM_006474 | duplicate | |
| podoplanin | | | | |
| cellular retinol-binding protein | M11433 | NM_002899 | SEQ ID NO: 67 | SEQ ID NO: 68 |
| macrophage mannose receptor (MRC1) | M93221 | | SEQ ID NO: 69 | SEQ ID NO: 70 |
| transcription factor C-MAF | AF055376 | NM_005360 | SEQ ID NO: 71 | SEQ ID NO: 72 |
| transcription factor C-MAF | AF055376 | NM_005360 | duplicate | |
| selenoprotein P | Z11793 | NM_005410 | SEQ ID NO: 73 | SEQ ID NO: 74 |
| KIAA0466, immunoglobulin superfamily, | AB007935 | NM_001542 | SEQ ID NO: 75 | SEQ ID NO: 76 |
| member 3 | | | | |
| type II membrane protein similar to HIV gp120- | AB015629 | NM_014257 | SEQ ID NO: 77 | SEQ ID NO: 78 |
| binding C-type lectin, CD209 antigen-like | | | | |
| KIAA0626 | AB014526 | NM_021647 | SEQ ID NO: 79 | SEQ ID NO: 80 |
| KIAA0711 | AB018254 | NM_014867 | SEQ ID NO: 81 | SEQ ID NO: 82 |
| integrin alpha 9 | D25303 | NM_002207 | SEQ ID NO: 83 | SEQ ID NO: 84 |
| integrin alpha 9 | D25303 | NM_002207 | duplicate | |
| relaxin H2 | X00948 | NM_005059 | SEQ ID NO: 85 | SEQ ID NO: 86 |
| KIAA0644 | AB014544 | NM_014817 | SEQ ID NO: 87 | SEQ ID NO: 88 |
| Cdk-inhibitor p57KIP2 (KIP2) | U22398 | NM_000076 | SEQ ID NO: 89 | SEQ ID NO: 90 |
| Cdk-inhibitor p57KIP2 (KIP2) | U22398 | NM_000076 | duplicate | |
| transient receptor potential channel TRPC6 | AJ006276 | NM_004621 | SEQ ID NO: 91 | SEQ ID NO: 92 |
| cDNA DKFZp564O222 (from clone | AL050002 | | | SEQ ID NO: 93 |
| DKFZp564O222) | | | | |
| | M80482 | NM_002570 | SEQ ID NO: 94 | SEQ ID NO: 95 |
| regulator of G-protein signalling 16, A28-RGS14p | U70426 | NM_002928 | SEQ ID NO: 96 | SEQ ID NO: 97 |
| dihydropyrimidinase related protein-1, collapsin | D78012 | NM_001313 | SEQ ID NO: 98 | SEQ ID NO: 99 |
| response mediator protein 1 | | | | |
| desmoplakin (DPI, DPII) | AL031058 | NM_004415 | SEQ ID NO: 100 | SEQ ID NO: 101 |
| pendrin, solute carrier family, member 4 | AF030880 | NM_000441 | SEQ ID NO: 102 | SEQ ID NO: 103 |
| reelin (RELN) | U79716 | NM_005045 | SEQ ID NO: 104 | SEQ ID NO: 105 |
| integrin, alpha 1 | X68742 | | | SEQ ID NO: 106 |
| integrin alpha 1 | X68742 | | duplicate | |
| cholesterol 25-hydroxylase | AF059214 | NM_003956 | SEQ ID NO: 107 | SEQ ID NO: 108 |
| inhibin beta-B-subunit precursor | M31682 | NM_002193 | SEQ ID NO: 109 | SEQ ID NO: 110 |
| KIAA1233 | AL109724 | | | |
| pre-B cell stimulating factor homologue (SDF1b) | L36033 | NM_000609 | SEQ ID NO: 112 | SEQ ID NO: 113 |
| V-Erba Related Ear-3 Protein | HG3510-HT3704 | | | SEQ ID NO: 114 |
| antigen identified by monoclonal antibody MRC | X05323 | | SEQ ID NO: 115 | SEQ ID NO: 116 |
| OX-2 | | | | |
| apolipoprotein D | J02611 | NM_001647 | SEQ ID NO: 117 | SEQ ID NO: 118 |
| TIMP3, tissue inhibitor of matrix | U14394 | NM_000362 | SEQ ID NO: 119 | SEQ ID NO: 120 |
| metalloproteinases | | | | |
| TIMP3 | U14394 | NM_000362 | duplicate | |
| aldehyde dehydrogenase 1 | K03000 | NM_000689 | SEQ ID NO: 121 | SEQ ID NO: 122 |
| prospero-related homeobox 1 (prox 1) | U44060 | NM_002763 | SEQ ID NO: 123 | SEQ ID NO: 124 |
| matrix Gla protein | AI953789 | NM_000900 | SEQ ID NO: 125 | SEQ ID NO: 126 |
| neuronal pentraxin II (NPTX2) | U29195 | | SEQ ID NO: 127 | SEQ ID NO: 128 |
| histatin 2 (HIS2) | M26665 | NM_000200 | SEQ ID NO: 129 | SEQ ID NO: 130 |
| ADDL mRNA for adducin-like protein, adducin 3 | D67031 | NM_016824 | SEQ ID NO: 131 | SEQ ID NO: 132 |
| (gamma) | | | | |
| adducin 3 (gamma) | U37122 | NM_016824 | duplicate | |
| MADS box transcription enhancer factor 2, | L08895 | NM_002397 | SEQ ID NO: 133 | SEQ ID NO: 134 |
| polypeptide C (myocyte enhancer factor 2C) | | | | |
| MADS box transcription enhancer factor 2, | | NM_002397 | duplicate | |
| (myocyte enhancer factor 2C) | | | | |
| MADS box transcription enhancer factor 2, | S57212 | NM_002397 | duplicate | |
| polypeptide C (myocyte enhancer factor 2C) | | | | |
| phosphoglucomutase 5 | L40933 | NM_021965 | SEQ ID NO: 135 | SEQ ID NO: 136 |
| cyclin E2 | AF102778 | NM_004702 | SEQ ID NO: 137 | SEQ ID NO: 138 |
| interleukin 7 (IL7) | M29053 | | SEQ ID NO: 139 | SEQ ID NO: 140 |
| interleukin 7 | J04156 | NM_000880 | duplicate | |
| cDNA DKFZp586L0120 (from clone | AL050154 | | | SEQ ID NO: 141 |
| DKFZp586L0120) | | | | |
| peroxisome proliferative activated receptor, | L40904 | NM_005037 | SEQ ID NO: 142 | SEQ ID NO: 143 |
| gamma, PPARG | | | | |
| fatty acid binding protein 4 | AA128249 | NM_001442 | SEQ ID NO: 144 | SEQ ID NO: 145 |
| protein kinase C zeta | Z15108 | NM_002744 | SEQ ID NO: 146 | SEQ ID NO: 147 |
| 46 kDa coxsackievirus and adenovirus receptor | Y07593 | NM_001338 | SEQ ID NO: 148 | SEQ ID NO: 149 |
| (CAR) protein | | | | |
| PAC clone RP4-751H13 from 7q35-qter, zinc | AC004877 | | SEQ ID NO: 150 | SEQ ID NO: 151 |
| finger-like | | | | |
| thymidine kinase 1, soluble | M15205 | NM_003258 | SEQ ID NO: 152 | SEQ ID NO: 153 |
| thymidine kinase 1 | K02581 | NM_003258 | duplicate | |
| Pig7 (PIG7), LPS-induced TNF-alpha factor | AF010312 | NM_004862 | SEQ ID NO: 154 | SEQ ID NO: 155 |
| LPS-induced TNF-alpha factor | AL120815 | NM_004862 | duplicate | |
| lipase A, lysosomal acid, cholesterol esterase | X76488 | NM_000235 | SEQ ID NO: 156 | SEQ ID NO: 157 |
| ubiquitin specific protease 13 (isopeptidase T-3) | U75362 | NM_003940 | SEQ ID NO: 158 | SEQ ID NO: 159 |
| carcinoembryonic antigen-related cell adhesion | X16354 | NM_001712 | SEQ ID NO: 160 | SEQ ID NO: 161 |
| molecule 1 (biliary glycoprotein) CEACAM1 | | | | |
| cDNA DKFZp586D0918 (from clone | AL049370 | | | SEQ ID NO: 162 |
| DKFZp586D0918) | | | | |
| KIAA0598, B cell RAG associated protein | AB011170 | NM_014863 | SEQ ID NO: 163 | SEQ ID NO: 164 |
| RAMP2 (receptor (calcitonin) activity modifying | AJ001015 | NM_005854 | SEQ ID NO: 165 | SEQ ID NO: 166 |
| protein 2) | | | | |
| cholesteryl ester transfer protein precursor | M30185 | NM_000078 | SEQ ID NO: 167 | SEQ ID NO: 168 |
| epithelial membrane protein 2 | U52100 | NM_001424 | SEQ ID NO: 169 | SEQ ID NO: 170 |
| MHC class II lymphocyte antigen (HLA-DP) beta | M83664 | NM_002121 | SEQ ID NO: 171 | SEQ ID NO: 172 |
| chain | | | | |
| MHC class II lymphocyte antigen (HLA-DP) beta | M83664 | NM_002121 | duplicate | |
| chain | | | | |
| beta-arrestin 2 | AF106941 | NM_004313 | SEQ ID NO: 173 | SEQ ID NO: 174 |
| mitotic checkpoint kinase Bub1 (BUB1) | AF053305 | NM_004336 | SEQ ID NO: 175 | SEQ ID NO: 176 |
| KIAA0229, similar to human ankyrin 1(S08275) | D86982 | | SEQ ID NO: 177 | SEQ ID NO: 178 |
| Sprouty 1 homolog (antagonist of FGF signaling) | AF041037 | | SEQ ID NO: 179 | SEQ ID NO: 180 |
| guanine nucleotide exchange factor for Rap1; M- | | NM_012294 | SEQ ID NO: 181 | SEQ ID NO: 182 |
| Ras-regulated GEF, KIAA0277 | | | | |
| translin | X78627 | NM_004622 | SEQ ID NO: 183 | SEQ ID NO: 184 |
| erythrocyte membrane protein band 4.9 (dematin) | U28389 | NM_001978 | SEQ ID NO: 185 | SEQ ID NO: 186 |
| KIAA0846 protein | AB020653 | NM_015376 | SEQ ID NO: 187 | SEQ ID NO: 188 |
| glia maturation factor, gamma | W07033 | NM_004877 | SEQ ID NO: 189 | SEQ ID NO: 190 |
| insulin-like growth factor binding protein 2 | X16302 | NM_000597 | SEQ ID NO: 191 | SEQ ID NO: 192 |
| (IGFBP-2) | | | | |
| smooth muscle myosin heavy chain isoform | S67247 | | SEQ ID NO: 193 | SEQ ID NO: 194 |
| Smemb | | | | |
| TTG-2 (cysteine rich protein with LIM motif), | X61118 | NM_005574 | SEQ ID NO: 195 | SEQ ID NO: 196 |
| LIM domain only 2 (rhombotin-like 1) | | | | |
| cyclin B2 | AL080146 | NM_004701 | SEQ ID NO: 197 | SEQ ID NO: 198 |
| KIAA0353 | AB002351 | | SEQ ID NO: 199 | SEQ ID NO: 200 |
| KIAA0559, piccolo (presynaptic cytomatrix | AB011131 | | SEQ ID NO: 201 | SEQ ID NO: 202 |
| G protein-coupled receptor, family C, group 5, | AC004131 | NM_016235 | SEQ ID NO: 203 | SEQ ID NO: 204 |
| G protein-coupled receptor, family C, group 5, | AI801872 | NM_016235 | duplicate | dup |
| member B | | | | |
| CREM (cyclic AMP-responsive element | S68134 | NM_001881 | SEQ ID NO: 205 | SEQ ID NO: 206 |
| modulator beta isoform) | | | | |
| CREM (cyclic AMP-responsive element | S68134 | NM_001881 | duplicate | dup |
| modulator beta isoform) | | | | |
| CREM (cyclic AMP-responsive element | S68271 | NM_001881 | duplicate | dup |
| modulator beta isoform) | | | | |
| hypothetical protein FLJ13110 | AL080222 | NM_022912 | SEQ ID NO: 207 | SEQ ID NO: 208 |
| inositol(myo)-1(or 4)-monophosphatase 2 | AF014398 | NM_014214 | SEQ ID NO: 209 | SEQ ID NO: 210 |
| KIAA0937 protein | AB023154 | | SEQ ID NO: 211 | SEQ ID NO: 212 |
| mitotic spindle coiled-coil related protein | AF063308 | NM_006461 | SEQ ID NO: 213 | SEQ ID NO: 214 |
| cysteine and glycine-rich protein 2 (CSRP2) | U57646 | NM_001321 | SEQ ID NO: 215 | SEQ ID NO: 216 |
| topoisomerase (DNA) II alpha (170 kD) | AI375913 | NM_001067 | SEQ ID NO: 217 | SEQ ID NO: 218 |
| DNA topoisomerase II | J04088 | NM_001067 | duplicate | dup |
| protein phosphatase inhibitor 2 (PPP1R2) | U68111 | | SEQ ID NO: 219 | SEQ ID NO: 220 |
| KIAA0186 | D80008 | NM_021067 | SEQ ID NO: 221 | SEQ ID NO: 222 |
| dual-specificity tyrosine-(Y)-phosphorylation | Y12735 | NM_003582 | SEQ ID NO: 223 | SEQ ID NO: 224 |
| regulated kinase 3 (Dyrk3) | | | | |
| kinesin-like spindle protein HKSP (HKSP) | U37426 | NM_004523 | SEQ ID NO: 225 | SEQ ID NO: 226 |
| huntingtin-associated protein interacting protein | U94190 | NM_003947 | SEQ ID NO: 227 | SEQ ID NO: 228 |
| (duo) | | | | |
| diubiquitin | AL031983 | NM_006398 | SEQ ID NO: 229 | SEQ ID NO: 230 |
| bikunin, serine protease inhibitor, Kunitz type, 2 | U78095 | NM_021102 | SEQ ID NO: 231 | SEQ ID NO: 232 |
| cytochrome P-450-1 (TCDD-inducible) | K03191 | NM_000499 | SEQ ID NO: 233 | SEQ ID NO: 234 |
| cytochrome P(1)-450 | X02612 | NM_000499 | duplicate | dup |
| KIAA0513 | | NM_014732 | SEQ ID NO: 235 | SEQ ID NO: 236 |
| protein phosphatase inhibitor 2 (PPP1R2) | U68111 | | duplicate | |
| RAMP3 (receptor (calcitonin) activity modifying | AJ001016 | NM_005856 | SEQ ID NO: 237 | SEQ ID NO: 238 |
| protein 3) | | | | |
| B-myb | X13293 | NM_002466 | SEQ ID NO: 239 | SEQ ID NO: 240 |
| KIAA0952 | AB023169 | NM_014962 | SEQ ID NO: 241 | SEQ ID NO: 242 |
| interferon stimulated gene (20 kD), HEM45 | U88964 | NM_002201 | SEQ ID NO: 243 | SEQ ID NO: 244 |
| GS3955 | D87119 | NM_021643 | SEQ ID NO: 245 | SEQ ID NO: 246 |
| GS3955 | D87119 | NM_021643 | duplicate | dup |
| GRB2-related adaptor protein (Grap) | U52518 | NM_006613 | SEQ ID NO: 247 | SEQ ID NO: 248 |
| KIAA1071 protein | AB028994 | | SEQ ID NO: 249 | SEQ ID NO: 250 |
| RNA-binding protein gene with multiple splicing, | D84111 | NM_006867 | SEQ ID NO: 251 | SEQ ID NO: 252 |
| RBP-MS/type 5 | | | | |
| RNA-binding protein gene with multiple splicing, | D84111 | NM_006867 | duplicate | dup |
| RBP-MS/type 5 | | | | |
| RBP-MS/type 4, RNA-binding protein gene with | D84110 | NM_006867 | duplicate | |
| multiple splicing | | | | |
| RBP-MS/type 4, RNA-binding protein gene with | D84110 | NM_006867 | duplicate | dup |
| multiple splicing | | | | |
| RBP-MS/type 3, RNA-binding protein gene with | D84109 | NM_006867 | duplicate | dup |
| multiple splicing | | | | |
| alpha-actinin-2-associated LIM protein | AF002282 | NM_014476 | SEQ ID NO: 253 | SEQ ID NO: 254 |
| semaphorin-III (Hsema-I), semaphorin 3A | L26081 | NM_006080 | SEQ ID NO: 255 | SEQ ID NO: 256 |
| IQ motif containing GTPase activating protein 2 | U51903 | NM_006633 | SEQ ID NO: 257 | SEQ ID NO: 258 |
| Arrestin, Beta 2 | HG2059-HT2114 | | duplicate | |
| retinoblastoma-associated protein HEC | AF017790 | NM_006101 | SEQ ID NO: 259 | SEQ ID NO: 260 |
| LIM domain binding protein (LDB1) | AF052389 | NM_001290 | SEQ ID NO: 261 | SEQ ID NO: 262 |
| dual specificity phosphatase 5 | U15932 | NM_004419 | SEQ ID NO: 263 | SEQ ID NO: 264 |
| Homo sapiens cDNA 3′, mRNA sequence | AI557322 | | | SEQ ID NO: 265 |
| monoamine oxidase A (MAOA) | M68840 | NM_000240 | SEQ ID NO: 266 | SEQ ID NO: 267 |
| monoamine oxidase A | AA420624 | NM_000240 | duplicate | |
| NECDIN related protein | U35139 | NM_002487 | SEQ ID NO: 268 | SEQ ID NO: 269 |
| regulatory solute carrier protein, family 1, | X82877 | NM_006511 | SEQ ID NO: 270 | SEQ ID NO: 271 |
| member 1 | | | | |
| TTK protein kinase | M86699 | NM_003318 | SEQ ID NO: 272 | SEQ ID NO: 273 |
| fms-related tyrosine kinase 4, VEGFR-3 | X69878 | NM_002020 | SEQ ID NO: 274 | SEQ ID NO: 275 |
| TSC403, similar to lysosome-associated | AB013924 | NM_014398 | SEQ ID NO: 276 | SEQ ID NO: 277 |
| membrane glycoprotein | | | | |
| HMG-2 | X62534 | | SEQ ID NO: 278 | SEQ ID NO: 279 |
| Homo sapiens clone 24416 mRNA sequence | AF052159 | | SEQ ID NO: 280 | SEQ ID NO: 281 |
| calcitonin receptor-like | L76380 | NM_005795 | SEQ ID NO: 282 | SEQ ID NO: 283 |
| KIAA0582 protein | AI761647 | NM_015147 | SEQ ID NO: 284 | SEQ ID NO: 285 |
| cDNA DKFZp434B102 (from clone | AL080192 | | | SEQ ID NO: 286 |
| DKFZp434B102) | | | | |
| cDNA DKFZp586G1922 (from clone | AL080110 | | SEQ ID NO: 287 | SEQ ID NO: 287 |
| DKFZp586G1922) | | | | |
| Acyl-CoA synthetase 3 | D89053 | NM_004457 | SEQ ID NO: 288 | SEQ ID NO: 289 |
| fatty-acid-Coenzyme A ligase, long-chain 3 | AA977580 | NM_004457 | duplicate | |
| STAT induced STAT inhibitor-2 | AF037989 | | SEQ ID NO: 290 | SEQ ID NO: 291 |
| Homeotic Protein Hox5.4 | HG3502-HT3696 | | | SEQ ID NO: 292 |
| hypothetical protein FLJ13910, cDNA | AL050139 | NM_022780 | SEQ ID NO: 293 | SEQ ID NO: 294 |
| DKFZp586M141 (from clone DKFZp586M141) | | | | |
| cDNA DKFZp586N012 (from clone | AL049471 | | | SEQ ID NO: 295 |
| DKFZp586N012) | | | | |
| UbcH10, ubiquitin carrier protein E2-C | U73379 | NM_007019 | SEQ ID NO: 296 | SEQ ID NO: 297 |
| cyclin-dependent kinase inhibitor 3, protein | L25876 | NM_005192 | SEQ ID NO: 298 | SEQ ID NO: 299 |
| tyrosine phosphatase (CIP2) | | | | |
| glycogen phosphorylase (PYGL) | AF046798 | | SEQ ID NO: 300 | SEQ ID NO: 301 |
| Angiopoietin-2 | AF004327 | NM_001147 | SEQ ID NO: 302 | SEQ ID NO: 303 |
| Angiopoietin-2 | AF004327 | NM_001147 | duplicate | dup |
| forkhead box M1 | U74612 | NM_021953 | SEQ ID NO: 304 | SEQ ID NO: 305 |
| potentially prenylated protein tyrosine | AF041434 | NM_007079 | SEQ ID NO: 306 | SEQ ID NO: 307 |
| phosphatase hPRL-3 | | | | |
| RAB31, Low Mr GTP-binding protein of the Rab | U59877 | NM_006868 | SEQ ID NO: 308 | SEQ ID NO: 309 |
| subfamily | | | | |
| RAB31, member RAS oncogene family | AI189226 | NM_006868 | | |
| myosin VIIA | U39226 | NM_000260 | SEQ ID NO: 310 | SEQ ID NO: 311 |
| Grb2-associated binder-1, docking protein related | U43885 | NM_002039 | SEQ ID NO: 312 | SEQ ID NO: 313 |
| to IRS-1 | | | | |
| lamin B1 | L37747 | | SEQ ID NO: 314 | SEQ ID NO: 315 |
| minichromosome maintenance deficient (mis5, S. pombe) | D84557 | NM_005915 | SEQ ID NO: 316 | SEQ ID NO: 317 |
| 6 HsMcm6 | | | | |
| cyclin B1 | M25753 | | | SEQ ID NO: 318 |
| cyclin B1 | M25753 | | duplicate | dup |
| RTP, N-myc downstream regulated | D87953 | NM_006096 | SEQ ID NO: 319 | SEQ ID NO: 320 |
| alpha2,3-sialyltransferase | AB022918 | NM_006100 | SEQ ID NO: 321 | SEQ ID NO: 322 |
| ADP-ribosylation factor-like protein 4 | U73960 | NM_005738 | SEQ ID NO: 323 | SEQ ID NO: 324 |
| centromere protein F (350/400 kD, mitosin) | U30872 | NM_016343 | SEQ ID NO: 325 | SEQ ID NO: 326 |
| paternally expressed 10, KIAA1051 | AB028974 | NM_015068 | SEQ ID NO: 327 | SEQ ID NO: 328 |
| tubulin, alpha 1 (testis specific) | X06956 | | SEQ ID NO: 329 | SEQ ID NO: 330 |
| KIAA0101 | D14657 | NM_014736 | SEQ ID NO: 331 | SEQ ID NO: 332 |
| KIAA0128, septin 2 | D50918 | | SEQ ID NO: 333 | SEQ ID NO: 334 |
| protein phosphatase 2, regulatory subunit B (B56), | Z69030 | NM_002719 | SEQ ID NO: 335 | SEQ ID NO: 336 |
| gamma | | | | |
| deoxycytidine kinase | M60527 | NM_000788 | SEQ ID NO: 337 | SEQ ID NO: 338 |
| integrin beta 3 binding protein (beta3-endonexin) | U37139 | NM_014288 | SEQ ID NO: 339 | SEQ ID NO: 340 |
| TAL1 (SCL) interrupting locus | M74558 | NM_003035 | SEQ ID NO: 341 | SEQ ID NO: 342 |
| KIAA0666 | AB014566 | | SEQ ID NO: 343 | SEQ ID NO: 344 |
| cAMP-specific phosphodiesterase 8A, PDE8A1 | AF056490 | | SEQ ID NO: 345 | SEQ ID NO: 346 |
| mitotic checkpoint kinase Mad3L (MAD3L), | AF053306 | NM_001211 | SEQ ID NO: 347 | SEQ ID NO: 348 |
| BUB1B | | | | |
| ribosomal S6 kinase | X85106 | NM_021135 | SEQ ID NO: 349 | SEQ ID NO: 350 |
| HPTP epsilon (protein tyrosine phosphatase | X54134 | NM_006504 | SEQ ID NO: 351 | SEQ ID NO: 352 |
| epsilon) | | | | |
| Lyn tyrosine kinase, v-yes-1 Yamaguchi sarcoma | M79321 | NM_002350 | SEQ ID NO: 353 | SEQ ID NO: 354 |
| viral related oncogene homolog | | | | |
| lyn tyrosine kinase, v-yes-1 Yamaguchi sarcoma | M16038 | NM_002350 | duplicate | |
| viral related oncogene homolog | | | | |
| lyn tyrosine kinase | M16038 | NM_002350 | duplicate | |
| brachyury variant A (TBX1), T-box 1 | AF012130 | NM_005992 | SEQ ID NO: 355 | SEQ ID NO: 356 |
| transcription factor | | | | |
| mki67a mRNA (long type) for antigen of | X65550 | NM_002417 | SEQ ID NO: 357 | SEQ ID NO: 358 |
| monoclonal antibody Ki-67 | | | | |
| protein tyrosine phosphatase receptor pi (PTPRP) | U81561 | NM_002847 | SEQ ID NO: 359 | SEQ ID NO: 360 |
| cb1-b | U26710 | NM_004351 | SEQ ID NO: 361 | SEQ ID NO: 362 |
| Cyclin A2 | X51688 | NM_001237 | SEQ ID NO: 363 | SEQ ID NO: 364 |
| nucleoside phosphorylase | X00737 | NM_000270 | SEQ ID NO: 365 | SEQ ID NO: 366 |
| TNF-related apoptosis inducing ligand TRAIL | U37518 | NM_003810 | SEQ ID NO: 367 | SEQ ID NO: 368 |
| phosphodiesterase 4B, cAMP-specific | L20971 | NM_002600 | SEQ ID NO: 369 | SEQ ID NO: 370 |
| nidogen (enactin) | M30269 | NM_002508 | SEQ ID NO: 371 | SEQ ID NO: 372 |
| HYA22 protein | D88153 | NM_005808 | SEQ ID NO: 373 | SEQ ID NO: 374 |
| phosphatidic acid phosphatase type 2A | AF014402 | NM_003711 | SEQ ID NO: 375 | SEQ ID NO: 376 |
| KIAA0512, ALEX2 | AB011084 | NM_014782 | SEQ ID NO: 377 | SEQ ID NO: 378 |
| thromboxane A2 receptor | D38081 | NM_001060 | SEQ ID NO: 379 | SEQ ID NO: 380 |
| trefoil factor 3 (intestinal) | AI985964 | NM_003226 | SEQ ID NO: 381 | SEQ ID NO: 382 |
| G-2 and S-phase expressed 1 | AL031588 | NM_016426 | SEQ ID NO: 383 | SEQ ID NO: 384 |
| ADP-ribosyltransferase (NAD+; poly (ADP- | AJ236876 | NM_005484 | SEQ ID NO: 385 | SEQ ID NO: 386 |
| ribose) polymerase)-like 2 | | | | |
| serine/threonine kinase 12 | AF015254 | NM_004217 | SEQ ID NO: 387 | SEQ ID NO: 388 |
| Tubulin, Alpha 1, Isoform 44 | HG2259-HT2348 | | duplicate | |
| lamin B receptor | L25931 | NM_002296 | SEQ ID NO: 389 | SEQ ID NO: 390 |
| KIAA0429 | AB007889 | NM_014751 | SEQ ID NO: 391 | SEQ ID NO: 392 |
| transcription factor 4 | M74719 | NM_003199 | SEQ ID NO: 393 | SEQ ID NO: 394 |
| syndecan 3 (N-syndecan), KIAA0468 | AB007937 | NM_014654 | SEQ ID NO: 395 | SEQ ID NO: 396 |
| RECK protein precursor | AA099265 | NM_021111 | SEQ ID NO: 397 | SEQ ID NO: 398 |
| Putative prostate cancer tumor suppressor | U42349 | NM_006765 | SEQ ID NO: 399 | SEQ ID NO: 400 |
| protein phosphatase 1, regulatory (inhibitor) | AB020630 | | SEQ ID NO: 401 | SEQ ID NO: 402 |
| subunit | | | | |
| PDZ and LIM domain 1 (elfin) | U90878 | NM_020992 | SEQ ID NO: 403 | SEQ ID NO: 404 |
| hypothetical protein from clone 643 | AF091087 | NM_020467 | SEQ ID NO: 405 | SEQ ID NO: 406 |
| p53-regulated DDA3 | AA926959 | | | SEQ ID NO: 407 |
| KIAA0062 | D31887 | | SEQ ID NO: 408 | SEQ ID NO: 409 |
| medium-chain acyl-CoA dehydrogenase | M91432 | | SEQ ID NO: 410 | SEQ ID NO: 411 |
| gap junction protein, alpha 1, 43 kD (connexin 43) | M65188 | NM_000165 | SEQ ID NO: 412 | SEQ ID NO: 413 |
| MyoD family inhibitor | U78313 | NM_005586 | SEQ ID NO: 414 | SEQ ID NO: 415 |
| endo/exonuclease Mre11 (MRE11A) | AF073362 | NM_005591 | SEQ ID NO: 416 | SEQ ID NO: 417 |
| nuclear receptor subfamily 2, group F, member 1 | X16155 | NM_005654 | SEQ ID NO: 418 | SEQ ID NO: 419 |

TABLE 15
Sequence identifiers for sequences in Table 4

| | accession | | aa SEQ ID | nt SEQ ID |
|---|---|---|---|---|
| | numbers | | NO: | NO: |
| p27 mRNA, interferon alpha- | X67325 | NM_005532 | 420 | 421 |
| inducible protein 27 | | | | |
| ribonuclease A (RNase A), | D26129 | NM_002933 | 422 | 423 |
| pancreatic | | | | |
| hematopoietic neural membrane | U87947 | NM_001425 | 424 | 425 |
| protein (HNMP-1) | | | | |
| N-cadherin | M34064 | NM_001792 | 426 | 427 |
| N-cadherin | M34064 | NM_001792 | duplicate | |
| interleukin 8 (IL8) | M28130 | NM_000584 | 428 | 429 |
| interleukin 8, beta-thromboglobulin- | M17017 | NM_000584 | 430 | 431 |
| like protein precursor | | | | |
| tyrosine kinase receptor (axl) | M76125 | NM_001699 | 432 | 433 |
| | HG162-HT3165 | | duplicate | |
| cell surface glycoprotein CD44 | L05424 | | 434 | 435 |
| (CD44) | | | | |
| cell adhesion molecule (CD44) | M59040 | NM_000610 | duplicate | |
| hyaluronate receptor (CD44) | L05424 | | duplicate | |
| vascular endothelial growth factor | U43142 | NM_005429 | 436 | 437 |
| related protein VRP, VEGF-C | | | | |
| Vascular endothelial growth factor C | X94216 | NM_005429 | duplicate | |
| collagen type XIII, alpha 1 | M33653 | NM_005203 | 438 | 439 |
| (=COL4A2) | | | | |
| collagen type XIII, alpha-1 | M59217 | NM_005203 | duplicate | |
| collagen alpha-2 type I | K01079 | | | 440 |
| collagen alpha-2 type I | K01079 | | duplicate | |
| collagen, type I, alpha 2 | V00503 | NM_000089 | duplicate | |
| proteoglycan 1 | X17042 | NM_002727 | 441 | 442 |
| phospholipase A2, group IV A, | M72393 | | 443 | 444 |
| calcium-dependent phospholipid- | | | | |
| binding protein (PLA2) | | | | |
| carbohydrate (keratan sulfate Gal-6) | AB003791 | NM_003654 | 445 | 446 |
| sulfotransferase | | | | |
| tropomyosin 2 (beta), fibroblast | M12125 | NM_003289 | 447 | 448 |
| tropomyosin | | | | |
| chondroitin sulfate proteoglycan 2 | X15998 | NM_004385 | 449 | 450 |
| (versican) | | | | |
| chondroitin sulfate proteoglycan 2 | X15998 | NM_004385 | duplicate | |
| (versican) | | | | |
| latent transforming growth factor- | Z37976 | NM_000428 | 451 | 452 |
| beta binding protein (LTBP-2) | | | | |
| interleukin 6 (interferon, beta 2) | X04430 | NM_000600 | 453 | 454 |
| bone morphogenetic protein-4 | U43842 | NM_001202 | 455 | 456 |
| (hBMP-4) | | | | |
| bone morphogenetic protein 2B, | M22490 | NM_001202 | duplicate | |
| BMP-4 | | | | |
| sarcolectin, keratin 7 | AJ238246 | NM_005556 | 457 | 458 |
| neuronal cell adhesion molecule, | AB002341 | NM_005010 | 459 | 460 |
| KIAA0343 | | | | |
| neuronal cell adhesion molecule, | U55258 | NM_005010 | duplicate | |
| hBRAVO/Nr-CAM precursor | | | | |
| matrix metalloproteinase 1 | M13509 | NM_002421 | 461 | 462 |
| (interstitial collagenase), skin | | | | |
| collagenase | | | | |
| stem cell factor, KIT ligand | M59964 | NM_000899 | 463 | 464 |
| uPA | X02419 | NM_002658 | 465 | 466 |
| plasminogen activator inhibitor-1 | J03764 | NM_000602 | 467 | 468 |
| plasminogen activator inhibitor 1 | M14083 | NM_000602 | duplicate | |
| selectin P, CD62, granule membrane | M25322 | NM_003005 | 469 | 470 |
| protein-140 (GMP-140) precursor | | | | |
| latrophilin-2 | AJ131581 | NM_012302 | 471 | 472 |
| actin, alpha 2 | X13839 | NM_001613 | 473 | 474 |
| fibroblast activation protein, alpha | U09278 | NM_004460 | 475 | 476 |
| regulator of G-protein signalling 20 | AF060877 | NM_003702 | 477 | 478 |
| IGF-II mRNA-binding protein 3 | U97188 | NM_006547 | 479 | 480 |
| retina cDNA randomly primed | W28438 | | | 481 |
| sublibrary, EST | | | | |
| brain acid-soluble protein 1, | AF039656 | NM_006317 | 482 | 483 |
| neuronal tissue-enriched acidic | | | | |
| protein (NAP-22) | | | | |
| profilin 2 | AL096719 | NM_002628 | 484 | 485 |
| profilin 2 | L10678 | NM_002628 | duplicate | |
| Na,K-ATPase beta-1 subunit | U16799 | NM_001677 | 486 | 487 |
| Claudin-7 | AJ011497 | NM_001307 | 488 | 489 |
| normal gingiva | U51712 | | | 490 |
| a disintegrin and metalloproteinase | AB009672 | NM_003812 | 491 | 492 |
| domain 23 | | | | |
| COL8A1 mRNA for alpha 1(VIII) | X57527 | NM_001850 | 493 | 494 |
| collagen | | | | |
| signal transducer and activator of | AF067575 | | 495 | 496 |
| transcription 6 (STAT6) | | | | |
| transcription factor IL-4 Stat, | U16031 | NM_003153 | duplicate | |
| STAT6 | | | | |
| lipocortin-III, annexin A3 | M20560 | NM_005139 | 497 | 498 |
| intercellular adhesion molecule 1 | M24283 | NM_000201 | 499 | 500 |
| (CD54), major group rhinovirus | | | | |
| receptor precusor | | | | |
| solute carrier family 1 | U08989 | NM_004170 | 501 | 502 |
| (neuronal/epithelial high affinity | | | | |
| glutamate transporter, system Xag) | | | | |
| solute carrier family 1 | AI928365 | NM_004170 | duplicate | |
| (neuronal/epithelial high affinity | | | | |
| glutamate transporter, system Xag) | | | | |
| p53 inducible protein | L47738 | | 503 | 504 |
| dihydropyrimidine dehydrogenase, | U20938 | NM_000110 | 505 | 506 |
| DPYD | | | | |
| natural killer cell transcript 4 | AA631972 | NM_004221 | 507 | 508 |
| PFTAIRE protein kinase 1, | AB020641 | NM_012395 | 509 | 510 |
| KIAA0834 | | | | |
| RGP4, regulator of G-protein | U27768 | NM_005613 | 511 | 512 |
| signalling 4 | | | | |
| regulator of G-protein signalling 4 | AI267373 | NM_005613 | duplicate | |
| Oncogene Amll-Evi-1, Fusion | HG4058-HT4328 | | 513 | 514 |
| Activated | | | | |
| Oncogene Amll-Evi-1, Fusion | HG4058-HT4328 | | duplicate | |
| Activated | | | | |
| adenylyl cyclase-associated protein 2 | N90755 | NM_006366 | 515 | 516 |
| clusterin (complement lysis | M25915 | NM_001831 | 517 | 518 |
| inhibitor, SP-40, 40, sulfated | | | | |
| glycoprotein 2, apolipoprotein J) | | | | |
| ADP ribosylation factor-like 7 | AB016811 | NM_005737 | 519 | 520 |
| H factor (complement)-like 1 | M65292 | NM_002113 | 521 | 522 |
| RNA helicase-related protein, | H68340 | NM_007372 | 523 | 524 |
| metallothionein-If | | | | |
| stimulated trans-acting factor (50 kDa) | X82200 | NM_006074 | 525 | 526 |
| Staf50 | | | | |
| cyclooxygenase-2 (hCox-2) | U04636 | NM_000963 | 527 | 528 |
| GRO1 oncogene, melanoma growth | X54489 | NM_001511 | 529 | 530 |
| stimulatory activity (MGSA) | | | | |
| NRGN, neurogranin | X99076 | NM_006176 | 531 | 532 |
| homologue of mouse dkk-1 | AB020315 | | 533 | 534 |
| gastrointestinal tumor-associated | J04152 | NM_002353 | 535 | 536 |
| antigen GA733-1, tumor-associated | | | | |
| calcium signal transducer 2 | | | | |
| laminin | Z15008 | NM_005562 | 537 | 538 |
| transgelin, 22 kDa smooth muscle | M95787 | NM_003186 | 539 | 540 |
| protein (SM22) | | | | |
| JE gene encoding a monocyte | M28225 | | 541 | 542 |
| secretory protein | | | | |
| zinc finger protein 238, RP58 | AJ223321 | NM_006352 | 543 | 544 |
| cathepsin C | X87212 | NM_001814 | 545 | 546 |
| tissue-type plasminogen activator (t- | M15518 | NM_000930 | 547 | 548 |
| PA) | | | | |
| sushi-repeat protein | AF060567 | NM_014467 | 549 | 550 |
| annexin A6 | D00510 | NM_001155 | 551 | 552 |
| EphrinB1 | U09303 | NM_004429 | 553 | 554 |
| EphrinB1 | U09303 | NM_004429 | duplicate | |
| TFEC isoform (transcription factor | D43945 | NM_012252 | 555 | 556 |
| EC) | | | | |
| small inducible cytokine A2, | M26683 | NM_002982 | 557 | 558 |
| (monocyte chemotactic protein1) | | | | |
| small inducible cytokine A2 | M26683 | NM_002982 | duplicate | |
| (monocyte chemotactic protein 1) | | | | |
| endothelial cell protein C/APC | L35545 | NM_006404 | 559 | 560 |
| receptor (EPCR) | | | | |
| transglutaminase 2 (TGase) | M55153 | NM_004613 | 561 | 562 |
| transglutaminase (TGase) | M55153 | NM_004613 | duplicate | |
| human metallothionein-If | M10943 | | 563 | 564 |
| transforming growth factor beta- | M77349 | NM_000358 | 565 | 566 |
| induced (BIGH3) | | | | |
| ENO2 gene for neuron specific | X51956 | | 567 | 568 |
| (gamma) enolase | | | | |
| FAT tumor suppressor (Drosophila) | X87241 | NM_005245 | 569 | 570 |
| homolog | | | | |
| malignant cell expression-enhanced | S82470 | NM_024298 | 571 | 572 |
| gene/tumor progression-enhanced | | | | |
| gene | | | | |
| malignant cell expression-enhanced | S82470 | NM_024298 | duplicate | 574 |
| gene/tumor progression-enhanced | | | | |
| gene | | | | |
| cDNA DKFZp566G0746 (from | AL050078 | | | 575 |
| clone DKFZp566G0746) | | | | |
| lysyl oxidase-like 2 | U89942 | NM_002318 | 576 | 577 |
| ras-related C3 botulinum toxin | M64595 | NM_002872 | 578 | 579 |
| substrate 2 (rho family, small GTP | | | | |
| binding protein Rac2) | | | | |
| endothelial leukocyte adhesion | M24736 | NM_000450 | 580 | 581 |
| molecule 1 (ELAM-1), selectin E | | | | |
| laminin, alpha 5, KIAA0533 | AB011105 | | 582 | 583 |
| placenta growth factor (PIGF) | X54936 | NM_002632 | 584 | 585 |
| ALL1-fused gene from chromosome | U16954 | NM_006818 | 586 | 587 |
| 1q, AF1q | | | | |
| stromelysin-2, MMP-10 | X07820 | NM_002425 | 588 | 589 |
| metallothionein-I-A | K01383 | | 590 | 591 |
| collagen VI alpha-1 | X15880 | | 592 | 593 |
| mad protein homolog (hMAD-3) | U68019 | NM_005902 | 594 | 595 |
| mad protein homolog (hMAD-3) | U68019 | NM_005902 | duplicate | |
| mad protein homolog (hMAD-3) | U68019 | NM_005902 | duplicate | |
| integral membrane protein 2A | AL021786 | | | 596 |
| interleukin 1 receptor-like 1 | D12763 | NM_003856 | 597 | 598 |
| high-mobility group (nonhistone | X92518 | | 599 | 600 |
| chromosomal) protein isoform I-C | | | | |
| (HMGI-C) | | | | |
| epidermal growth factor receptor | U12535 | NM_004447 | 601 | 602 |
| kinase substrate (Eps8) | | | | |
| lactate dehydrogenase B | X13794 | NM_002300 | 603 | 604 |
| mRNA for unknown product | D29810 | | 605 | 606 |
| hypothetical protein | AL033377 | | | 607 |
| DKFZp564D0462 | | | | |
| lysyl hydroxylase isoform 2 | U84573 | NM_000935 | 608 | 609 |
| (PLOD2) | | | | |
| follistatin-like 3, follistatin-related | U76702 | NM_005860 | 610 | 611 |
| protein (FLRG) | | | | |
| Homo sapiens clone 24674 mRNA | AF070578 | | | 612 |
| sequence | | | | |
| L-iditol-2 dehydrogenase | L29254 | | 613 | 614 |
| neuronal pentraxin 1 | U61849 | NM_002522 | 615 | 616 |
| hypothetical protein from clones | U90908 | NM_021226 | 617 | 618 |
| 23549 and 23762 | | | | |
| UDP-N-acetylglucosamine | AB011004 | NM_003115 | 619 | 620 |
| pyrophosphorylase | | | | |
| zinc finger protein 185 (LIM | Y09538 | NM_007150 | 621 | 622 |
| domain) | | | | |
| four and a half LIM domains 2, | U29332 | NM_001450 | 623 | 624 |
| heart protein (FHL-2) | | | | |
| mitogen-activated protein kinase- | U09578 | NM_004635 | 625 | 626 |
| activated protein kinase 3, | | | | |
| MAPKAP kinase (3pK) | | | | |
| metallothionein 1E (functional) | R92331 | | | 627 |
| TU3A protein | AF035283 | NM_007177 | 628 | 629 |
| metallothionein 1H | R93527 | NM_005951 | 630 | 631 |
| guanylate binding protein isoform II | M55543 | NM_004120 | 632 | 633 |
| (GBP-2) | | | | |
| soluble vascular endothelial cell | U01134 | NM_002019 | 634 | 635 |
| growth factor receptor 1 (sVEGFR- | | | | |
| 1) | | | | |
| R-Ras | M14949 | | 636 | 637 |
| R-ras | M14949 | | 638 | 639 |
| creatine transporter (SLC6A8), | U36341 | NM_005629 | 640 | 641 |
| solute carrier family 6, member 8 | | | | |
| target of myb1 (chicken) homolog, | Z82244 | NM_005488 | 642 | 643 |
| Heme Oxygenase 1 (HO-1) | | | | |
| procollagen-lysine, 2-oxoglutarate | L06419 | NM_000302 | 644 | 645 |
| 5-dioxygenase, lysyl hydroxylase | | | | |
| (PLOD) | | | | |
| KIAA0836 | AB020643 | | 646 | 647 |
| cDNA DKFZp434C171 (from clone | AL080169 | | 648 | 649 |
| DKFZp434C171) | | | | |
| IL-4-R mRNA for the interleukin 4 | X52425 | NM_000418 | 650 | 651 |
| receptor | | | | |
| chemokine (C—C motif) receptor- | AF014958 | NM_003965 | 652 | 653 |
| like 2 (CCRL2), chemokine receptor | | | | |
| X (CKRX) | | | | |
| phospholipase C, beta 3 | Z16411 | NM_000932 | 654 | 655 |
| (phosphatidylinositol-specific) | | | | |
| LIM domain protein | X93510 | NM_003687 | 656 | 657 |
| protein kinase (cAMP-dependent, | M34181 | NM_002731 | 658 | 659 |
| catalytic) inhibitor beta | | | | |
| rho GDP-dissociation Inhibitor 2 | X69549 | NM_001175 | 660 | 661 |
| KIAA0975, imidazoline receptor | AB023192 | NM_007184 | 662 | 663 |
| candidate | | | | |
| poliovirus receptor | X64116 | NM_006505 | 664 | 665 |
| poliovirus receptor | X64116 | NM_006505 | duplicate | |
| immediate early response 3 | S81914 | NM_003897 | 666 | 667 |
| metallothionein 2A | AI547258 | NM_005953 | 668 | 669 |
| tropomyosin 1 (alpha) | M19267 | NM_000366 | 670 | 671 |
| tropomyosin 1 (alpha) | Z24727 | NM_000366 | duplicate | |
| tropomyosin 1 (alpha) | M19267 | NM_000366 | duplicate | |
| TRAM-like protein | D31762 | NM_012288 | 672 | 673 |
| E3 ubiquitin ligase SMURF2 | AA630312 | NM_022739 | 674 | 675 |
| EGF-containing fibulin-like | U03877 | NM_004105 | 676 | 677 |
| extracellular matrix protein 1 | | | | |
| G protein-coupled receptor 56 | AJ011001 | NM_005682 | 678 | 679 |
| c-jun proto oncogene (JUN) | J04111 | NM_002228 | 680 | 681 |
| regulator of G-protein signalling 10, | AF045229 | NM_002925 | 682 | 683 |
| RGS10 | | | | |
| amyloid beta (A4) precursor | U62325 | | 684 | 685 |
| protein-binding, family B, member | | | | |
| 2 (Fe65-like) | | | | |
| ras-related rho protein | M12174 | NM_004040 | 686 | 687 |
| proteasome (prosome, macropain) | AL031177 | NM_002814 | 688 | 689 |
| 26S subunit, non-ATPase, 10 | | | | |
| KIAA0537 | AB011109 | NM_014840 | 690 | 691 |
| lysosome-associated membrane | X77196 | NM_002294 | 692 | 693 |
| protein-2 | | | | |
| phospholipid transfer protein | L26232 | NM_006227 | 694 | 695 |
| N-myristoyltransferase 2 | AF043325 | NM_004808 | 696 | 697 |
| phosphofructokinase (PFKM) | U24183 | NM_000289 | 698 | 699 |
| integrin, beta 4 | X53587 | NM_000213 | 700 | 701 |
| leupaxin | AF062075 | NM_004811 | 702 | 703 |
| endothelin-converting-enzyme 1 | Z35307 | NM_001397 | 704 | 705 |
| wild-type p53 activated fragment-1 | U03106 | NM_000389 | 706 | 707 |
| (WAF1), cyclin-dependent kinase | | | | |
| inhibitor 1A (p21, Cip1) | | | | |
| ICAM-2, cell adhesion ligand for | X15606 | NM_000873 | 708 | 709 |
| LFA-1 | | | | |
| ICAM-2, cell adhesion ligand for | X15606 | NM_000873 | duplicate | |
| LFA-1 | | | | |
| intercellular adhesion molecule 2 | M32334 | | 710 | 711 |
| (ICAM-2) | | | | |
| eukaryotic translation initiation | AF035280 | NM_014239 | 712 | 713 |
| factor 2B, eIF-2B beta subunit | | | | |
| uridine phosphorylase | X90858 | NM_003364 | 714 | 715 |
| integrin, beta 5 | X53002 | NM_002213 | 716 | 717 |
| N-sulfoglucosamine sulfohydrolase | U30894 | NM_000199 | 718 | 719 |
| (sulfamidase) | | | | |
| synaptojanin 2 | AF039945 | | 720 | 721 |
| metallothionein 1L | AA224832 | NM_002450 | 722 | 723 |
| macrophage capping protein, | M94345 | NM_001747 | 724 | 725 |
| gelsolin-like | | | | |
| HSPC022 protein | W68830 | NM_014029 | 726 | 727 |
| Human clone 137308 mRNA, | AW006742 | | no | 728 |
| partial cds | | | | |
| protocadherin 42, PC42, | L11370 | NM_002587 | 729 | 730 |
| protocadherin 1 (cadherin-like 1) | | | | |
| caspase-like apoptosis regulatory | AF005775 | NM_003879 | 731 | 732 |
| protein 2 (CLARP2) | | | | |
| caspase-like apoptosis regulatory | AF005775 | NM_003879 | duplicate | |
| protein 2 (CLARP2) | | | | |
| major vault protein, lrp | X79882 | NM_005115 | 733 | 734 |
| Fanconi anemia, complementation | AC004472 | NM_004629 | 735 | 736 |
| group G | | | | |
| prion protein (PrP) | U29185 | NM_000311 | 737 | 738 |
| interferon-stimulated protein, 15 kDa | AA203213 | NM_005101 | 739 | 740 |
| serine (or cysteine) proteinase | L40377 | NM_002640 | 741 | 742 |
| inhibitor, clade B (ovalbumin), | | | | |
| cytoplasmic antiproteinase 2 | | | | |
| (CAP2) | | | | |
| biglycan | J04599 | NM_001711 | 743 | 744 |
| chemokine (C—X—C motif), receptor | L06797 | NM_003467 | 745 | 746 |
| 4 (fusin) | | | | |
| ubiquitin carboxyl-terminal esterase | X04741 | NM_004181 | 747 | 748 |
| L1 (ubiquitin thiolesterase) | | | | |
| KIAA0469 | AB007938 | NM_014851 | 749 | 750 |
| TNF (ligand) superfamily, member | AL022310 | NM_003326 | 751 | 752 |
| 4 (tax-transcriptionally activated | | | | |
| glycoprotein 1, 34 kD) | | | | |
| KIAA1053 | AB028976 | | 753 | 754 |
| NAD(P)H-quinone oxireductase | M81600 | | 755 | 756 |
| sushi-repeat-containing protein | U61374 | NM_006307 | 757 | 758 |
| integrin, alpha 5 | X06256 | NM_002205 | 759 | 760 |
| enigma (LIM domain protein) | L35240 | NM_005451 | 761 | 762 |
| ectonucleoside triphosphate | AJ133133 | NM_001776 | 763 | 764 |
| diphosphohydrolase 1 | | | | |
| transforming growth factor-beta | M60315 | NM_001718 | 765 | 766 |
| (tgf-beta), bone morphogenetic | | | | |
| protein 6 | | | | |
| transforming growth factor-beta | M60315 | NM_001718 | duplicate | |
| (tgf-beta), bone morphogenetic | | | | |
| protein 6 | | | | |
| nicotinamide N-methyltransferase, | U08021 | NM_006169 | 767 | 768 |
| NNMT | | | | |
| cDNA DKFZp564J0323 (from | AL049957 | | no | 769 |
| clone DKFZp564J0323) | | | | |
| thioredoxin reductase beta | AB019694 | NM_006440 | 770 | 771 |
| f-box and leucine-rich repeat protein 2 | AL049953 | | 772 | 773 |
| transcobalamin II (TCN2) | L02648 | NM_000355 | 774 | 775 |
| aldehyde dehydrogenase 2, | X05409 | NM_000690 | 776 | 777 |
| mitochondrial | | | | |
| GTP-binding protein ragB | X90530 | NM_006064 | 778 | 779 |
| lymphocyte antigen 75 | AF011333 | NM_002349 | 780 | 781 |
| GM2 activator protein | X62078 | | 782 | 783 |
| type 3 inositol 1,4,5-trisphosphate | U01062 | NM_002224 | 784 | 785 |
| receptor (ITPR3) | | | | |
| KIAA0284 | AI828210 | | no | 786 |
| metallothionein I-B | M13485 | | 787 | 788 |
| BTG2 | U72649 | NM_006763 | 789 | 790 |
| adenylate kinase 1 | J04809 | NM_000476 | 791 | 792 |
| tumor necrosis factor receptor | Y09392 | NM_003790 | 793 | 794 |
| superfamily, member 12, WSL-LR, | | | | |
| WSL-S1 and WSL-S2 proteins | | | | |
| aminopeptidase N/CD13 | M22324 | NM_001150 | 795 | 796 |
| growth arrest and DNA-damage- | M60974 | NM_001924 | 797 | 798 |
| inducible protein (gadd45) | | | | |
| KIAA0638 protein | AB014538 | | 799 | 800 |
| vinculin | M33308 | NM_003373 | 801 | 802 |
| procollagen-proline, 2-oxoglutarate | U90441 | NM_004199 | 803 | 804 |
| 4-dioxygenase (proline 4- | | | | |
| hydroxlase), alpha polypeptide II | | | | |
| msg1-related gene 1 (mrg1), | U65093 | NM_006079 | 805 | 806 |
| Cbp/p300-interacting transactivator | | | | |
| microsomal glutathione S- | AF026977 | NM_004528 | 807 | 808 |
| transferase 3 | | | | |
| vitamin A responsive; cytoskeleton | AF070523 | NM_006407 | 809 | 810 |
| related | | | | |
| 17-kDa protein, interferon- | M13755 | NM_005101 | 811 | 812 |
| stimulated protein, 15 kDa | | | | |
| matrix metalloproteinase 14 | X83535 | NM_004995 | 813 | 814 |
| (membrane-inserted) | | | | |
| 4F2 cell-surface antigen, solute | J02939 | NM_002394 | 815 | 816 |
| carrier family 3, member 2 | | | | |
| metallothionein-III | M93311 | NM_005954 | 817 | 818 |
| protein kinase (cAMP-dependent, | S76965 | NM_006823 | 819 | 820 |
| catalytic) inhibitor alpha | | | | |
| protein kinase (cAMP-dependent, | S76965 | NM_006823 | duplicate | |
| catalytic) inhibitor alpha | | | | |
| reticulocalbin 1, EF-hand calcium | D42073 | NM_002901 | 821 | 822 |
| binding domain | | | | |
| lipin 1, KIAA0188 | D80010 | | 823 | 824 |
| protease, serine, 23 | AF015287 | NM_007173 | 825 | 826 |
| hect domain and RLD 2 | AF041080 | NM_004667 | 827 | 828 |
| GATA-binding protein (GATA2) | M68891 | NM_002050 | 829 | 830 |
| agrin precursor | AF016903 | | 831 | 832 |
| equilibrative nucleoside transporter | U81375 | NM_004955 | 833 | 834 |
| 1 (hENT1) | | | | |
| coronin, actin-binding protein 2B, | AB023142 | | 835 | 836 |
| KIAA0925 | | | | |
| f-box and WD-40 domain protein 3 | U07000 | NM_012165 | 837 | 838 |
| nonsyndromic hearing impairment | AF073308 | NM_004403 | 839 | 840 |
| protein (DFNA5) | | | | |
| actin filament associated protein | D25248 | NM_021638 | 841 | 842 |
| TNFR-related death receptor-6 | AF068868 | NM_014452 | 843 | 844 |
| (DR6) | | | | |
| serum/glucocorticoid regulated | Y10032 | NM_005627 | 845 | 846 |
| kinase | | | | |
| DNase X | X90392 | NM_006730 | 847 | 848 |
| DNase X | X90392 | NM_006730 | duplicate | |
| fatty acid desaturase 3 | AC004770 | NM_021727 | 849 | 850 |
| LYL-1 | M22637 | | 851 | 852 |
| ATP-binding cassette, sub-family C | X78338 | NM_004996 | 853 | 854 |
| (CFTR/MRP), member 1 | | | | |
| transmembrane protein (CD59) | M84349 | | 855 | 856 |
| fms-related tyrosine kinase 1, | S77812 | | 857 | 858 |
| VEGFR-1 | | | | |
| Hypothetical protein FLJ23403 | AI681538 | NM_022068 | 859 | 860 |
| hypothetical protein FLJ20898 | AI733570 | NM_024600 | 861 | 862 |

1. A method for differentially modulating the growth or differentiation of blood endothelial cells (BEC) or lymphatic endothelial cells (LEC), comprising contacting endothelial cells with a composition comprising an agent that differentially modulates blood or lymphatic endothelial cells, said agent selected from the group consisting of:
(a) a polypeptide that comprises an amino acid sequence of a BEC polypeptide or a LEC polypeptide, or an active fragment of said polypeptide;
(b) a polynucleotide that comprises a nucleotide sequence that encodes a polypeptide according to (a);
(c) an antibody that specifically binds to a polypeptide according to (a);
(d) a polypeptide comprising a fragment of the-antibody of (c), wherein the fragment and the antibody bind to the polypeptide;
(e) an antisense nucleic acid to a human gene or mRNA encoding the polypeptide of (a);
(f) an interfering RNA (RNAi) to a human gene or mRNA encoding the polypeptide of (a).

2. A method according to claim 1, wherein the endothelial cells are contacted with the composition ex vivo.

3. A method according to claim 1, wherein the composition comprises a pharmaceutically acceptable diluent, adjuvant, or carrier, and the contacting step comprises administering the composition to a mammalian subject to differentially modulate BECs or LECs in the mammalian subject.

4. A method according to claim 3, comprising:
identifying a human subject with a disorder characterized by hyperproliferation of LECs; and
administering to the human subject the composition, wherein the agent differentially inhibits LEC growth compared to BEC growth.

5. A method according to claim 3, comprising:
identifying a human subject with a disorder characterized by hyperproliferation of LECs;
screening LECs of the subject to identify overexpression of a polypeptide set forth in Table 3; and
administering to the human subject the composition, wherein the agent differentially inhibits LEC growth compared to BEC growth by inhibiting expression of the polypeptide identified by the screening step.

6. A method according to claim 3 of modulating the growth of lymphatic endothelial cells in a human subject, comprising steps of:
identifying a human subject with a hypoproliferative lymphatic disorder;
screening the subject to identify underexpression or underactivity of a LEC polypeptide set forth in Table 3, wherein said protein is not set forth in Table 1 or 2;
administering to the human subject said composition, wherein the agent comprises the LEC polypeptide (a) identified by the screening step or an active fragment of said polypeptide, or comprises the polynucleotide (b) that comprises a nucleotide sequence that encodes the polypeptide.

7. Use of an agent for the manufacture of a medicament for the differential modulation of blood vessel endothelial cell (BEC) or lymphatic vessel endothelial cell (LEC) growth or differentiation, said agent selected from the group consisting of:
(a) a polypeptide that comprises an amino acid sequence of a BEC polypeptide or a LEC polypeptide, or an active fragment of said polypeptide;
(b) a polynucleotide that comprises a nucleotide sequence that encodes a polypeptide according to (a);
(c) an antibody that specifically binds to a polypeptide according to (a);
(d) a polypeptide comprising a fragment of the antibody of (c), wherein the fragment and the antibody bind to the polypeptide;
(e) an antisense nucleic acid to a human gene or mRNA encoding the polypeptide of (a);
(f) an interfering RNA (RNAi) to a human gene or mRNA encoding the polypeptide of (a).

8. A method or use according to claim 1, wherein the polypeptide is a LEC polypeptide selected from the LEC polypeptides set forth in Table 3, and the agent differentially modulates LEC growth or differentiation over BEC growth or differentiation.

9. A method or use according to claim 1, wherein the polypeptide is a BEC polypeptide selected from the BEC polypeptides set forth in Table 4, and the agent differentially modulates BEC growth or differentiation over LEC growth or differentiation.

10. A method or use according to claim 8, wherein the polypeptide is not set forth in Tables 1 or 2.

11. A method or use according to claim 8, wherein the LEC polypeptide comprises an amino acid sequence selected from the group consisting of SEQ ID NOs: 81, 187, 207, 211, 221, 235, 241, 293, and 391.

12. A method or use according to claim 8, wherein the LEC polypeptide comprises an amino acid sequence selected from the group consisting of SEQ ID NOs: 31-34, 46, and 48.

13. A method or use according to claim 12, wherein the agent comprises an antibody according to (c) or polypeptide according to (d).

14. A method according to claim 12, wherein the agent comprises an extracellular domain fragment of the polypeptide of (a), or a polynucleotide encoding said extracellular domain fragment.

15. A method or use according to claim 1, wherein the agent comprises an antisense molecule.

16. A method of treating hereditary lymphedema comprising:
identifying a human subject with lymphedema and with a mutation in at least one allele of a gene encoding a LEC protein identified in Table 3, wherein the mutation correlates with lymphedema in human subjects, and with the proviso that said LEC protein is not VEGFR-3; and
administering to said subject a composition comprising a lymphatic growth agent selected from the group consisting of VEGF-C polypeptides, VEGF-D polypeptides, VEGF-C polynucleotides, and VEGF-D polynucleotides.

17. Use of a lymphatic growth agent selected from the group consisting of VEGF-C polypeptides, VEGF-D polypeptides, VEGF-C polynucleotides, and VEGF-D polynucleotides in the manufacture of a medicament for the treatment of hereditary lymphedema resulting from a mutation in a LEC gene identified in Table 3, with the proviso that said gene is not VEGFR-3.

18. A method of screening for an endothelial cell disorder or predisposition to said disorder, comprising
obtaining a biological sample containing endothelial cell mRNA from a human subject; and
measuring expression of a BEC or LEC gene from the amount of mRNA in the sample transcribed from said gene, wherein the BEC or LEC gene encodes a polypeptide identified in Table 3 or 4.

19. A method of monitoring the efficacy or toxicity of a drug on endothelial cells, comprising steps of:
measuring expression of at least one BEC or LEC gene in endothelial cells of a mammalian subject before and after administering a drug to the subject, wherein the at least one BEC or LEC gene encodes a polypeptide set forth in Table 3 or Table 4, and wherein changes in expression of the BEC or LEC gene correlates with efficacy or toxicity of the drug on endothelial cells.

20. A method of identifying compounds that modulate growth of endothelial cells, comprising
culturing endothelial cells in the presence and absence of a compound; and
measuring expression of at least one BEC or LEC gene in the cells, wherein the BEC or LEC gene is selected from the genes encoding polypeptides set forth in Tables 3 and 4, wherein a change in expression of at least one BEC gene in the presence compared to the absence of the compound identifies the compound as a modulator of BEC growth, and wherein a change in expression of at least one LEC gene in the presence compared to the absence of the compound identifies the compound as a modulator of LEC growth.

21. A method according to claim 20 of screening for a compound that selectively modulates BEC or LEC growth or differentiation,
wherein the measuring step comprises measuring expression of at least one BEC gene and at least one LEC gene in the cells, and
wherein the method comprises screening for a compound that selectively modulates BEC or LEC growth or differentiation by selecting a compound that differentially modulates expression of the at least one BEC gene compared to expression of the at least one LEC gene.

22. A composition comprising
an isolated polynucleotide that comprises a nucleotide sequence that encodes a polypeptide comprising an amino acid sequence selected from the group consisting of SEQ ID NOs: 31-44, 46, 48, 50, 52, 81, 187, 207, 211, 221, 235, 241, 293, and 391; and
a pharmaceutically acceptable diluent, carrier or adjuvant.

23. A composition according to claim 22, comprising a polynucleotide that comprises a nucleotide sequence selected from the group consisting of SEQ ID NOS: 14-30, 45, 47, 49, 51, 82, 93, 111, 188, 208, 212, 222, 236, 242, 294, and 392, or a fragment thereof that encodes the polypeptide.

24. An expression vector comprising an expression control sequence operably linked to a polynucleotide that comprises a nucleotide sequence that encodes a polypeptide comprising an amino acid sequence selected from the group consisting of SEQ ID NOs: 31-44, 46, 48, 50, 52, 81, 187, 207, 211, 221, 235, 241, 293, and 391.

25. An expression vector according to claim 24 that is a replication-deficient adenoviral or adeno-associated viral vector containing the polynucleotide.

26. A composition comprising an expression vector according to claim 24 and a pharmaceutically acceptable diluent, carrier, or adjuvant.

27. A kit comprising the composition according to claim 22 packaged with a protocol for administering the composition to a mammalian subject to modulate the lymphatic system in said subject.

28. A host cell transformed or transfected with an expression vector according to claim 24.

29. A method for producing a LEC polypeptide comprising steps of growing a host cell according to claim 28 under conditions in which the cell expresses the polypeptide encoded by the polynucleotide.

30. A purified and isolated polypeptide comprising an amino acid sequence selected from the group consisting of SEQ ID NOS: 31-44, 46, 48, 50, 52, 81, 187, 207, 211, 221, 235, 241, 293, and 391.

31. A purified and isolated polypeptide comprising an amino acid sequence selected from the group consisting of:
(a) SEQ ID NOS: 31-34, 46, 48, 207, 676, 859, and 861; and
(b) an extracellular domain fragment of at least 10 amino acids of an amino acid sequence of (a).

32. A purified and isolated, soluble polypeptide according to claim 31 comprising an extracellular domain fragment of an amino acid sequence selected from the group consisting of: SEQ ID NOS: 31-34, 46, 48, 207, 676, 859, and 861, wherein the polypeptide lacks any transmembrane domain.

33. A polypeptide according to claim 32 that lacks any intracellular domain.

34. A fusion protein comprising a polypeptide according to claim 32 fused to an immunoglobulin fragment comprising an immunoglobulin constant region.

35. A composition comprising a polypeptide or protein according to claim 30 and a pharmaceutically acceptable diluent, carrier or adjuvant.

36. A kit comprising the composition according to claim 35 and a protocol for administering said pharmaceutical composition to a mammalian subject to modulate the lymphatic system in said subject.

37. An antibody that specifically binds to a polypeptide according to claim 30.

38. An antibody according to claim 37 that is a humanized antibody.

39. A protein comprising an antigen binding domain of an antibody that specifically binds-to a polypeptide according to claim 30, wherein said protein specifically binds to said polypeptide.

40. A method of identifying a LEC nucleic acid comprising:
(a) contacting a biological sample containing a candidate LEC nucleic acid with a polynucleotide comprising a fragment of at least 14 contiguous nucleotides of a sequence selected from the group consisting of SEQ ID NOS:1-30, 45, 47, 49, 51, 82, 93, 111, 188, 208, 212, 236, 242, 294, and 392, or a complement thereof, under the following stringent hybridization conditions:
(i) hybridization at 42° C. for 20 hours in a solution containing 50% formamide, 5×SSPE, 5× Denhardt's solution, 0.1% SDS and 0.1 mg/ml denatured salmon sperm DNA, and
(ii) washing for 30 minutes at 65° C. in 1×SSC, 0.1% SDS; and
(b) detecting hybridization of said candidate LEC nucleic acid and said polynucleotide, thereby identifying a LEC nucleic acid.

41. A method of identifying a LEC protein comprising:
(a) contacting a biological sample containing a candidate LEC protein with a LEC protein binding partner selected from the group consisting of an antibody according to claim 37, under conditions suitable for binding therebetween; and
(b) detecting binding between said candidate LEC protein and said LEC binding partner, thereby identifying a LEC protein.

42. A method of identifying a LEC comprising:
(a) contacting a biological sample comprising cells with a LEC binding partner under conditions suitable for binding therebetween, wherein said LEC binding partner comprises an antibody that binds to a polypeptide comprising a sequence selected from the group consisting of SEQ ID NOS:31-34, 46, 48, 207, 676, 859, and 861, or comprises an antigen binding fragment of said antibody; and
(b) identifying a LEC by detecting binding between a cell and said LEC binding partner, where binding of the LEC binding partner to the cell identifies a LEC.

43. A method of assaying for risk of developing hereditary lymphedema, comprising.
(a) assaying nucleic acid of a human subject for a mutation that correlates with a hereditary lymphedema phenotype and alters the encoded amino acid sequence of at least one gene allele of the human subject when compared to the amino acid sequence of the polypeptide encoded by a corresponding wild-type gene allele, wherein the wild-type polypeptide is a polypeptide identified in Table 3.

44. A method of assaying for risk of developing hereditary lymphedema, comprising
(a) assaying nucleic acid of a human subject for a mutation that correlates with a hereditary lymphedema phenotype and alters the encoded amino acid sequence of at least one gene allele of the human subject when compared to the amino acid sequence of the polypeptide encoded by a corresponding wild-type gene allele, wherein the wild-type polypeptide comprises an amino acid sequence selected from the group consisting of SEQ ID NOS: 31-44, 46, 48, 52, 54, 207, 676, 859, and 861;
(b) correlating the presence or absence of said mutation in the nucleic acid to a risk of developing hereditary lymphedema, wherein the presence of said mutation in the nucleic acid correlates with an increased risk of developing hereditary lymphedema, and wherein the absence of said mutation in the nucleic acid correlates with no increased risk of developing hereditary lymphedema.

45. A method of assaying for risk of developing hereditary lymphedema, comprising
(a) assaying nucleic acid of a human subject for a mutation that alters the encoded amino-acid sequence of at least one transcription factor allele of the human subject and alters transcription modulation activity of the transcription factor polypeptide encoded by the allele, when compared to the transcription modulation activity of a transcription factor polypeptide encoded by a wild-type allele,
wherein the wild-type transcription factor polypeptide comprises an amino acid sequence selected from the group consisting of SEQ ID NO: 81, SEQ ID NO: 211, SEQ ID NO: 241, and transcription factors encoded by sequences in Table 5; and
(b) correlating the presence or absence of said mutation in the nucleic acid to a risk of developing hereditary lymphedema, wherein the presence of said mutation in the nucleic acid correlates with an increased risk of developing hereditary lymphedema, and wherein the absence of said mutation in the nucleic acid correlates with no increased risk of developing hereditary lymphedema.

46. The method according to claim 45 wherein said wild-type transcription factor allele comprises the Sox18 amino acid sequence set forth as SEQ ID NO:54.

47. The method according to claim 46 wherein the assaying identifies a mutation altering a transactivating or DNA binding domain amino acid sequence of the protein encoded by the Sox18 allele.

48. The method according to claim 46, wherein said mutation reduces transcriptional activation of a SOX18-responsive gene compared to transcriptional activation of said gene by wild-type SOX18.

49. A method of assaying for risk of developing hereditary lymphedema, comprising
(a) assaying nucleic acid of a human subject for a mutation that alters the encoded amino acid sequence of at least one LEC gene allele of the human subject and alters the, binding affinity of the adhesion polypeptide encoded by the LEC gene allele, when compared to the binding affinity of an adhesion polypeptide encoded by a wild-type allele,
wherein the wild-type adhesion polypeptide comprises an amino acid sequence selected from the group consisting of SEQ ID NOS:31-34, 46, 207, 676, 859, and 861; and
(b) correlating the presence or absence of said mutation in the nucleic acid to a risk of developing hereditary lymphedema, wherein the presence of said mutation in the nucleic acid correlates with an increased risk of developing hereditary lymphedema, and wherein the absence of said mutation in the nucleic acid correlates with no increased risk of developing hereditary lymphedema.

50. The method according to claim 43, wherein the assaying identifies the presence of the mutation, and the correlating step identifies the increased risk of said patient developing hereditary lymphedema.

51. A method of screening a human subject for an increased risk of developing hereditary lymphedema comprising assaying nucleic acid of a human subject for a mutation that alters the encoded amino acid sequence of at least one polypeptide comprising an amino acid sequence of Table 3.

52. A method of claim 51, wherein the polypeptide comprises an amino acid sequence selected from the group consisting of SEQ ID NOS: 31-44, 46, 48, 50, 52, and 54, 207, 676, 859, and 861 in a manner that correlates with the risk of developing hereditary lymphedema.

53. The method according to claim 52 wherein the polypeptide comprises the SOX18 amino acid sequence set forth in SEQ ID NO: 54.

54. The method according to claim 43 wherein said method comprises at least one procedure selected from the group consisting of:
(a) determining a nucleotide sequence of at least one codon of at least one polynucleotide of the human subject;
(b) performing a hybridization assay: to determine whether nucleic acid from the human subject has a nucleotide sequence identical to or different from one or more reference sequences;
(c) performing a polynucleotide migration assay to determine whether nucleic acid from the-human subject has a nucleotide sequence identical to or different from one or more reference sequences; and
(d) performing a restriction endonuclease digestion to determine whether nucleic acid from the human subject has a nucleotide sequence identical to or different from one or more reference sequences.

55. The method according to claim 43 wherein said method comprises: performing a polymerase chain reaction (PCR) to amplify nucleic acid comprising the coding sequence of said LEC polynucleotide, and determining nucleotide sequence of the amplified nucleic acid.

56. A method of screening for a hereditary lymphedema genotype in a human subject, comprising:
(a) providing a biological sample comprising nucleic acid from said subject, and
(b) analyzing said nucleic acid for the presence of a mutation altering the encoded amino acid sequence of the at least one allele of at least one gene in the human subject relative to a human gene encoding an amino acid sequence selected from the group consisting of SEQ ID NOs: 31-44, 46, 48, 50, 52, 54, 207, 676, 859, and 861, wherein the presence of a mutation altering the encoded amino acid sequence in the human subject in a manner that correlates with lymphedema in human subjects identifies a hereditary lymphedema genotype.

57. The method according to claim 56 wherein said biological sample is a cell sample.

58. The method according to claim 56 wherein said analyzing comprises sequencing a portion of said nucleic acid.

59. The method according to claim 56 wherein the human subject has a hereditary lymphedema genotype identified by the method of screening.

60. The method according to claim 49, wherein the at least one gene corresponds to the human Sox18 gene that encodes the amino acid sequence set forth in SEQ ID NO: 54.

61. A method of inhibiting lymphangiogenesis comprising
administering to a subject an inhibitor of a LEC transmembrane polypeptide,
wherein the LEC transmembrane polypeptide comprises an amino acid sequence selected from the group consisting of SEQ ID NOs: 31-34, 46 48, 207, 676, 859, and 861, and
wherein the inhibitor is selected from the group consisting of
(a) a soluble extracellular domain fragment of the LEC transmembrane polypeptide;
(b) an antibody that binds to the extracellular domain of the LEC transmembrane polypeptide;
(c) a polypeptide comprising an antigen binding domain of the antibody according to (b); and
(d) an antisense nucleic acid complementary to the nucleic acid encoding the LEC transmembrane polypeptide or its complement.

62. A method according to claim 61, wherein the inhibitor is a polypeptide comprising an extracellular domain fragment of an LEC polypeptide, wherein the sequence of said extracellular domain is selected from the group consisting of amino acids 1-152 of SEQ ID NO:31, amino acids 1-695 of SEQ ID NO:32 and amino acids 1-248 of SEQ ID NO:33.

63. The method according to claim 61 wherein said subject is a human containing a tumor.

64. A method for modulating lymphangiogenesis in a mammalian subject comprising: administering to a mammalian subject in need of modulation of lymphangiogenesis an antisense molecule to a LEC polynucleotide, in an amount effective to inhibit transcription or translation of the polypeptide encoded by the LEC polynucleotide, wherein the LEC polynucleotide comprises a nucleotide sequence selected from the group consisting of SEQ ID NOS: 14-30, 45, 47, 49, AND 51, 208, 677, 860, and 862.

65. A method of treating hereditary lymphedema, comprising:
(a) identifying a human subject with hereditary lymphedema and with a mutation that alters the encoded amino acid sequence of at least one polypeptide of the human subject, relative to the amino acid sequence of a polypeptide comprising an amino acid sequence selected from the group consisting of SEQ ID NOS: 31-44, 46, 48, 50, 52, 54, 207, 676, 859, and 861; and
(b) administering to said subject a lymphatic growth factor selected from the group consisting of a VEGF-C polynucleotide, a VEGF-C polypeptide, a VEGF-D polynucleotide, and a VEGF-D polypeptide.

66. A method of modulating the growth of endothelial cells or endothelial precursor cells, comprising contacting endothelial cells or endothelial precursor cells with a composition comprising an agent the modulates prox-1 transcription regulation in the cells, wherein the agent is selected from the group consisting of:
(a) a prox-1 polypeptide;
(b) a polynucleotide encoding a prox-1 polypeptide;
(c) an antisense molecule to prox-1.

67. A method according to claim 66, wherein the cells comprises cultured endothelial cells or endothelial precursor cells, and the contacting is performed ex vivo.

68. A method according to claim 67, wherein the contacting comprises including the agent in the culture medium.

69. A method according to claim 66, wherein the cells comprise endothelial precursor cells.

70. A method according to claim 66, wherein the cells are introduced into a mammalian subject after the contacting step.

71. A method according to claim 70, wherein the subject is human.

72. A method according to claim 71, wherein the human subject has a LEC disorder.

73. A method of increasing LEC function in a human subject, comprising:
isolating endothelial cells or endothelial precursor cells from a human subject;
transforming or transfecting the endothelial cells with an expression vector comprising a nucleotide sequence encoding a prox-1 polypeptide, to promote LEC differentiation and growth; and
administering the LEC cells to a human subject after the transforming or transfecting step.

74. A method according to claim 73, wherein the human subject of the isolating and administering steps is the same.

75. A method according to claim 73, wherein the human subject has lymphedema.

76. A method according to claim 73, wherein the vector and transforming or transfecting method are selected for transient expression of the prox-1.

77. A method according to claim 73, wherein the expression vector comprises a replication-deficient adenoviral vector.

78. An isolated polypeptide comprising an amino acid sequence at least 95% identical to amino acids 61-127 of SEQ ID NO: 31.

79. A polypeptide according to claim 78, comprising an amino acid sequence at least 95% identical to amino acids 30-152 of SEQ ID NO: 31.

80. A soluble polypeptide comprising a fragment of the amino acid sequence set forth in SEQ ID NO: 31, wherein said fragment lacks the transmembrane and intracellular amino acids of SEQ ID NO: 31.

81. An isolated polypeptide comprising at least one leucine-rich region of SEQ ID NO: 32.

82. An isolated polypeptide according to claim 81, wherein the polypeptide lacks transmembrane amino acids of SEQ ID NO: 32.

83. An isolated polypeptide comprising at least one leucine-rich region of SEQ ID NO: 33.

84. An isolated polypeptide according to claim 81, wherein the polypeptide lacks transmembrane amino acids of SEQ ID NO: 33.

85. An isolated polypeptide comprising an amino acid sequence at least 95% identical to a fragment of a polypeptide comprising the amino acid sequence set forth in SEQ ID NO: 111,
wherein said fragment includes at least one thrombospondin type I repeat sequence.

86. An isolated polypeptide according to claim 85, wherein said fragment includes the six thrombospondin type I repeat sequences of SEQ ID NO: 111.

87. An isolated polypeptide comprising an amino acid sequence at least 95% identical to a fragment of a polypeptide comprising the amino acid sequence set forth in SEQ ID NO: 111,
wherein said fragment includes at least one immunoglobulin C-2 type domain.

88. An isolated polypeptide according to claim 85, wherein said fragment includes the three immunoglobulin C-2 type domain sequences of SEQ ID NO: 111.

89. A fusion protein comprising a polypeptide according to claim 78 and a heterologous polypeptide.

90. An antibody that specifically binds to a polypeptide according to claim 78.

91. A polynucleotide comprising a nucleotide sequence that encodes a polypeptide according to claim 78.

92. An expression vector comprising a polynucleotide according to claim 91 operatively linked to an expression control sequence.

93. An expression vector according to claim 92 that is a replication deficient adenoviral vector.

94. A method or use according to claim 9, wherein the polypeptide is not set forth in Tables 1 or 2.

95. A method or use according to claim 1, wherein the agent comprises an antisense molecule, and wherein the polypeptide is not set forth in Tables 1 or 2.

96. The method according to claim 43, wherein the assaying identifies the presence of the mutation, and the correlating step identifies the increased risk of said patient developing hereditary lymphedema, and wherein said method comprises at least one procedure selected from the group consisting of:
(a) determining a nucleotide sequence of at least one codon of at least one polynucleotide of the human subject;
(b) performing a hybridization assay to determine whether nucleic acid from the human subject has a nucleotide sequence identical to or different from one or more reference sequences;
(c) performing a polynucleotide migration assay to determine whether nucleic acid from the human subject has a nucleotide sequence identical to or different from one or more reference sequences; and
(d) performing a restriction endonuclease digestion to determine whether nucleic acid from the human subject has a nucleotide sequence identical to or different from one or more reference sequences.

97. The method according to claim 43, wherein the assaying identifies the presence of the mutation, and the correlating step identifies the increased risk of said patient developing hereditary lymphedema, wherein said method comprises: performing a polymerase chain reaction (PCR) to amplify nucleic acid comprising the coding sequence of said LEC polynucleotide, and determining nucleotide sequence of the amplified nucleic acid

98. A method according to claim 73, wherein the human subject has lymphedema, and wherein the vector and transforming or transfecting method are selected for transient expression of the prox-1.

99. A method according to claim 73, wherein the human subject has lymphedema, and wherein the expression vector comprises a replication-deficient adenoviral vector.
